(19) United States
(12) Patent Application Publication
(10) Pub. No.: US 2024/0181041 A1
(43) Pub. Date: Jun. 6, 2024

(54) Adenovirus SARS-CoV-2 Vaccine

(71) Applicant: THE WISTAR INSTITUTE, Philadelphia, PA (US)

(72) Inventor: Hildegund C.J. Ertl, Philadelphia, PA (US)

(21) Appl. No.: 18/282,029

(22) PCT Filed: Mar. 16, 2022
(86) PCT No.: PCT/US22/20604

§ 371 Date: Sep. 14, 2023

Related U.S. Application Data
(60) Provisional application No. 63/161,871, filed on Mar. 16, 2021.
(60) Provisional application No. 63/273,987, filed on Oct. 31, 2021.

(51) Int. Cl. A61K 39/215; A61K 39/00; A61P 31/14; A61P 37/04

(52) U.S. Cl. CPC A61K 39/215; A61P 31/14; A61P 37/04; A61K 2039/5254; A61K 2039/5256; A61K 2039/545; A61K 2039/6075; A61K 2039/70

(57) ABSTRACT

The present invention includes methods and compositions useful for treating or preventing a coronavirus infection in a subject. In certain embodiments the treatment comprises adenoviral-based vaccines against SARS-CoV-2 viral proteins.

### CROSS-REFERENCE TO RELATED APPLICATIONS

The present application is entitled to priority under 35 U.S.C. § 119(e) to U.S. Provisional Patent Application No. 63/161,871, filed Mar. 16, 2021, and U.S. Provisional Patent Application No. 63/273,987, filed Oct. 31, 2021, both of which are hereby incorporated by reference in their entireties herein.

### BACKGROUND OF THE INVENTION

More than 112 million humans have been infected worldwide with severe acute respiratory syndrome coronavirus 2 (SARS-CoV-2) with over 2.47 million fatalities due to its disease, COVID-19. In the US over 75 million humans have been diagnosed with COVID-19 and over 900,000 have died. Most deaths occur in vulnerable populations such as the elderly and humans with underlying chronic diseases. Tragically, in the US Afro-Americans have disproportionally high fatality rates. To slow the spread of SARS-CoV-2 many citizens of the US were under restrictions on crowd sizes that have resulted in millions of lost jobs and significant economic disruption. Although there is mounting evidence that social distancing is slowing the spread of SARS-CoV-2, only an efficacious vaccine will allow lifting of isolation measures without risking a resurgence of cases.

A number of SARS-CoV-2 vaccines are in clinical testing, under emergency use authorization (EUA), or have been licensed, but past experiences with vaccines to other pathogens such as HIV-1, dengue virus, and respiratory syncytial virus suggest that not every vaccine strategy is efficacious, especially in those with a weakened immune system such as the aged. Furthermore, all current EUA-approved vaccines exclusively target the spike protein, which is already subject to mutations, which have been shown to limit the effectiveness of these vaccines.

Correlates of protection against SARS-CoV-2 remain ill-defined. The spike (S) glycoprotein, which is expressed as a trimer on the virus surface binds to ACE2, its cellular receptor. S protein-specific antibodies can neutralize the virus and are therefore assumed to protect humans from becoming infected. Studies with a previously circulating SARS virus have shown that after infections of humans antibody titers decline fairly rapidly within 3 years rendering individuals potentially susceptible to reinfection. In contrast, T cell responses are sustained in humans after SARS infections and pre-clinical studies showed that SARS-specific CD8+ T cells can blunt the severity of infection, accelerate virus clearance and reduce spreading.

As such there is a clear need in the art for effective vaccines for SARS-CoV-2 that affect long-term immunity involving both B and T cell responses and against multiple viral targets in addition to the Spike protein. The current invention addresses these needs.

### SUMMARY OF THE INVENTION

As described herein, the present invention relates to methods and compositions useful for treating or preventing a coronavirus infection in a subject. In certain embodiments the treatment comprises adenoviral-based vaccines against SARS-CoV-2 viral proteins.

In one aspect, the invention includes an immunogenic composition comprising an effective amount of a first adenoviral vector comprising a first nucleotide sequence encoding a first SARS-CoV-2 protein and a second adenoviral vector comprising a second nucleotide sequence encoding a second SARS-CoV-2 protein.

In certain embodiments, the first SARS-CoV-2 protein is SARS-CoV-2 spike (S) protein and the second SARS-CoV-2 protein is SARS-CoV-2 nucleocapsid (N) protein.

In certain embodiments, the nucleocapsid protein is fused to a domain of herpes simplex virus glycoprotein D, thereby forming a fusion protein.

In certain embodiments, the SARS-CoV-2 spike protein comprises SEQ ID NO: 2 or SEQ ID NO: 6, and the SARS-CoV-2 nucleocapsid protein comprises SEQ ID NO: 4.

In certain embodiments, the first nucleotide sequence comprises SEQ ID NO: 1 or SEQ ID NO: 5, and the second nucleotide sequence comprises SEQ ID NO: 3.

In certain embodiments, the first adenoviral vector and the second adenoviral vector are replication-defective.

In certain embodiments, the first adenoviral vector and the second adenoviral vector are of chimpanzee origin.

In certain embodiments, the first adenoviral vector is serotype-6 (AdC6) or serotype-7 (AdC7).

In certain embodiments, the second adenoviral vector is serotype-6 (AdC6) or serotype-7 (AdC7).

In certain embodiments, the first adenoviral vector and the second adenoviral vector are of the same serotype.

In certain embodiments, the first adenoviral vector and the second adenoviral vector are of different serotypes.

In certain embodiments, the immunogenic composition further comprises a pharmaceutically acceptable vehicle.

In another aspect, the invention includes a viral vector comprising a first nucleotide sequence encoding a first SARS-CoV-2 protein and a second nucleotide sequence encoding a second SARS-CoV-2 protein.

In certain embodiments, the first SARS-CoV-2 protein is SARS-CoV-2 spike (S) protein and the second SARS-CoV-2 protein is SARS-CoV-2 nucleocapsid (N) protein.

In certain embodiments, the nucleocapsid protein is fused to a domain of herpes simplex virus glycoprotein D, thereby forming a fusion protein.

In certain embodiments, the SARS-CoV-2 spike protein comprises SEQ ID NO: 2 or SEQ ID NO: 6, and the SARS-CoV-2 nucleocapsid protein comprises SEQ ID NO: 4.

In certain embodiments, the first nucleotide sequence comprises SEQ ID NO: 1 or SEQ ID NO: 5, and the second nucleotide sequence comprises SEQ ID NO: 3.

In certain embodiments, the viral vector is an adenoviral vector.

In certain embodiments, the adenoviral vector is replication-defective.

In certain embodiments, the adenoviral vector is of chimpanzee origin.

In certain embodiments, the adenoviral vector is serotype-6 (AdC6) or serotype-7 (AdC7).

In another aspect, the invention includes a method of stimulating an immune response in a subject, comprising administering to the subject an effective amount of the immunogenic composition or the viral vector of any one of the above aspects or embodiments, or any aspect or embodiment disclosed herein.

In certain embodiments, the immunogenic composition or the viral vector is delivered by a route selected from the group consisting of oral route, inhalation route, nasal route, nebulization route, intravenous injection, intraperitoneal injection, intramuscular injection, subcutaneous injection, transdermal injection, and any combination thereof.

In another aspect, the invention includes a method for treating or preventing a coronavirus infection in a subject, comprising administering to the subject an effective amount of the immunogenic composition or the viral vector of any one of the above aspects or embodiments, or any aspect or embodiment disclosed herein.

In certain embodiments, the immunogenic composition or the viral vector is delivered by a route selected from the group consisting of oral route, inhalation route, nasal route, nebulization route, intravenous injection, intraperitoneal injection, intramuscular injection, subcutaneous injection, transdermal injection, and any combination thereof.

In certain embodiments, the coronavirus infection is caused by the SARS-CoV-2 virus.

In another aspect, the invention includes a method of stimulating an immune response in a subject, the method comprising administering to the subject: (i) an effective amount of a first adenoviral vector comprising a first nucleotide sequence encoding a first SARS-CoV-2 protein; and (ii) a second adenoviral vector comprising a second nucleotide sequence encoding a second SARS-CoV-2 protein.

In another aspect, the invention includes a method for treating or preventing a coronavirus infection in a subject, the method comprising administering to the subject: (i) an effective amount of a first adenoviral vector comprising a first nucleotide sequence encoding a first SARS-CoV-2 protein; and (ii) a second adenoviral vector comprising a second nucleotide sequence encoding a second SARS-CoV-2 protein.

In certain embodiments, the first SARS-CoV-2 protein is SARS-CoV-2 spike (S) protein and the second SARS-CoV-2 protein is SARS-CoV-2 nucleocapsid (N) protein.

In certain embodiments, the nucleocapsid protein is fused to a domain of herpes simplex virus glycoprotein D, thereby forming a fusion protein.

In certain embodiments, the SARS-CoV-2 spike protein comprises SEQ ID NO: 2 or SEQ ID NO: 6, and the SARS-CoV-2 nucleocapsid protein comprises SEQ ID NO: 4.

In certain embodiments, the first nucleotide sequence comprises SEQ ID NO: 1 or SEQ ID NO: 5, and the second nucleotide sequence comprises SEQ ID NO: 3.

In certain embodiments, the first adenoviral vector and the second adenoviral vector are replication-defective.

In certain embodiments, the first adenoviral vector is serotype-6 (AdC6) or serotype-7 (AdC7).

In certain embodiments, the second adenoviral vector is serotype-6 (AdC6) or serotype-7 (AdC7).

In certain embodiments, the first adenoviral vector and the second adenoviral vector are of the same serotype.

In certain embodiments, the first adenoviral vector and the second adenoviral vector are of different serotypes.

In certain embodiments, administration of the first adenoviral vector and administration of the second adenoviral vector each independently comprises a route selected from the group consisting of oral route, inhalation route, nasal route, nebulization route, intravenous injection, intraperitoneal injection, intramuscular injection, subcutaneous injection, transdermal injection, and any combination thereof.

In certain embodiments, the first adenoviral vector is administered prior to the second adenoviral vector.

In certain embodiments, the second adenoviral vector is administered prior to the first adenoviral vector.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIG. 1 depicts a vector map of the pShuttle vector comprising the spike gene.

FIG. 2 is a map of the recombinant molecular clone of AdC6 carrying the spike expression cassette.

FIG. 3 is a map of the recombinant molecular clone of AdC7 carrying the spike expression cassette.

FIG. 4 is a Western blot of lysates of cells infected with the spike vectors. Concentration of lysate that were loaded into each lane are displayed along the top. The molecular weight marker is shown to the left.

FIG. 5 is a map of the pShuttle vector for the nucleocapsid gene.

FIG. 6 is a map of the recombinant molecular clone of AdC6 carrying the nucleocapsid expression cassette.

FIG. 7 is a map of the recombinant molecular clone of AdC7 carrying the nucleocapsid expression cassette.

FIG. 8 shows a Western blot of lysages of cells infected with the NCap vectors. Uninfected cells or cells infected with a vector expressing a fusion protein of NCap and herpes simplex virus glycoprotein D (gDNCap) were used as a control. The molecular weight marker is shown to the left. Asterisks highlight the nucleocapsid band to its left.

FIG. 9 shows diagrams of wildtype and AgeI Covid19 spike proteins.

FIG. 10 shows virus neutralizing antibody (VNA) titers of commercially available serum samples from humans that have been infected with SARS-CoV-2 (immune) as well as from SARS-CoV-2 naïve humans using a neutralization assay based on a vesicular stomatitis virus pseudotype with the spike protein of an early SARS-Co-V2 isolate.

FIG. 11A-FIG. 11F show the optical density data (OD) obtained by ELISA for sera of individual mice collected at the indicated time points. Data were corrected by subtraction of background OD values. FIGS. 11A, 11B, and 11C are graphs showing results obtained with AdC6-Spike. FIGS. 11D, 11E, and 11F are graphs showing results obtained with AdC7-Spike. FIG. 11A, FIG. 11D 10⁹ virus particles (vp) vector dose, boosted after 4 weeks, FIG. 11B, FIG. 11E 10¹⁰ vp vector dose, boosted after 6 weeks, FIG. 11C, FIG. 11E 5×10¹⁰ vp vector dose, boosted after 6 weeks. Stars on top of graph show significant difference for a given time point between results obtained after prime versus boost by multiple t-tests. Numbers of stars indicate level of difference (*) p value between 0.01-0.05, (**) p value between 0.001-0.01, (***) p value between 0.0001-0.001, (****) p value <0.0001.

FIG. 12 shows virus neutralizing antibody (VNA) titers for individual sera tested on BHK21/WI-2 cells. A dilution of a VSV pseudotyped with SARS-CoV-2 spike and expressing GFP in infected cells was incubated for 90 min with serially diluted sera. Each sample was tested in duplicate. The mixture was then transferred to BHK21/WI-2 cells. Cells infected with virus not treated with serum were used as controls. Numbers of fluorescent cells were read 20-24 hours under a fluorescent microscope. The higher dilution of serum that caused 50% inhibition of cell transduction determine the titer. Lines show median results.

FIG. 13 shows VNA titers for young and aged C57Bl/6 mice immunized with 2×10⁹ vp of the AdC7-Spike vector. Sera were tested 2 and 4 weeks later as described in legend to FIG. 10.

FIG. 14 shows frequencies of spleen-derived CD8+(upper left), CD44+CD8+(upper right) CD4+(lower left) or CD44+CD4+(lower right) cells over all cells carrying the corresponding marker producing IFN-γ in response to the two NCap peptide pools. Results obtained with medium instead of peptides were subtracted. Graphs show means for duplicate samples or individual spleens.

FIG. 15 shows frequencies of spleen-derived CD8+(left) or CD44+CD8+ cells over all cells carrying the corresponding marker producing IFN-γ in response to individual peptides of NCap. Results obtained with medium instead of peptides were subtracted. Graphs show results obtained for naïve mice and mice vaccinated with AdC7-NCap.

FIG. 16 shows frequencies of spleen-derived CD8+(left) or CD44+CD8+ cells over all cells carrying the corresponding marker producing IFN-γ in response to individual peptides of NCap. Results obtained with medium instead of peptides were subtracted. Graphs show results obtained for naïve mice and mice vaccinated with AdC6-gDNCap followed by a boost with AdC7-gDNCap given 4 or 8 weeks later.

FIG. 17A-FIG. 17D relate to expression of the transgene products. [A,B] FIGS. 17A and 17B are Western blots for AdC7-N and AdC6-N probed with an antibody to N [FIG. 17A] and AdC7-gDN and AdC6-gDN [FIG. 17B] probed with an antibody to gD. Ad vectors expressing an unrelated protein were used as controls. * indicates the N protein in its wild-type form or fused into gD. Protein loading was controlled for by a subsequent stain with an antibody to ß-actin (not shown). FIGS. 17C and 17D relate to liquid chromatography with tandem mass spectrometry (LC-MS/MS). The diagrams are graphical representations of the protein expressed by AdC7-gDN [FIG. 17C] and AdC6-gDN [FIG. 17D] from amino acid residue 1 to 419. Protein sequences identified by LC-MS/MS are indicated by the grey boxes. The black trace shows the MS/MS spectra count of the identified peptides relative to the peptide with the highest spectra count.

FIG. 18 shows T cell responses to the vaccine inserts. CD8⁺ and CD4⁺ T cell responses to the two peptide pools are shown as % IFN-g⁺ cells/over all cells of the subset. Statistical differences calculated by multiple t-tests are indicated with lines with stars above. (****)-p value <0.0001; (**)-p-value between 0.001-0.01.

FIGS. 19A-19F shows CD8⁺ T cell responses after priming. Frequencies of CD8⁺ T cell responses to peptides of N that carry potential epitopes are shown as % IFN-g⁺CD44⁺CD8⁺ cells/CD44⁺CD8⁺ cells. Background responses were subtracted. The upper graphs show frequencies, the lower graphs show the relative distribution of responses to individual peptides. The numbers below the pie charts show the sum of frequencies (excluding those of positive adjacent peptides that according to epitope prediction express the same epitope) as indicators for response magnitude; legends indicate the peptide number to which responses were elicited. Those close to the pie charts were used to derive the sum of the response, those to the right were excluded. FIG. 19A, FIG. 19D: Responses to the AdC-Nap vector; FIG. 19B, FIG. 19E: responses to the AdC-gDN vector. For both sets of data mice were immunized with 2×10¹⁰ vp of vector and splenocytes were analysed 2 weeks later. FIG. 19C and FIG. 19F show responses to the AdC6-gDN vector given at 1×10¹⁰ vp/mouse. Splenocytes were analysed 3 months later.

FIGS. 20A-20F show CD4⁺ T cell responses after priming. The graphs show frequencies and relative distribution of responses to individual peptides as in FIGS. 19A-19F for mice immunized with 2×10¹⁰ vp of the AdC6-N (FIG. 20A, FIG. 20D) or AdC6-gDN (FIG. 20B, FIG. 20E) vectors analysed 2 weeks later or to 1×10¹⁰ vp of the AdC6-gDN vector (FIG. 20C, FIG. 20F) analysed 3 months later.

FIGS. 21A-21D show CD8⁺ T cell responses to AdC-N prime-boost regimens The graphs show frequencies and relative distributions of CD8⁺ T cells to individual peptides as in FIGS. 19A-19F using splenocytes from mice that had been primed with AdC6-N at 5 or 2×10¹⁰ vp and were boosted 6 weeks [FIG. 21A, FIG. 21C] or 2 months [FIG. 21B, FIG. 21D] later with the corresponding AdC7 vector. An additional group was primed with 1×10¹⁰ vp of AdC7-N and boosted 2 months later with AdC6-N. All assays were conducted 2 weeks after the boost.

FIGS. 22A-22H show CD8⁺ T cell responses to AdC-gDN prime-boost regimens. The graphs show frequencies and relative distribution of CD8⁺ T cells to individual peptides as in FIGS. 19A-19F using splenocytes from mice that had been primed with AdC7-gDN [FIGS. 22A, 22C, 22E, 22G] or AdC6-gDN [FIGS. 22B, 22F, 22D, 22H] at the indicated doses. They were boosted 4 to 8 weeks later as shown in the graph titles with the same dose of the corresponding heterologous vector. Splenocytes were tested 2 [FIGS. 22A, 22E] or 6 [FIGS. 22C, 22G] weeks or 3 [FIGS. 22B, 22F] or 4 [FIGS. 22D, 22H] months later.

FIGS. 23A-23H show CD4⁺ T cell responses to AdC-gDN prime-boost regimens. The graphs show frequencies and relative distribution of CD4⁺ T cells to individual peptides as in FIG. 3 using splenocytes from mice that had been primed with AdC7-gDN [FIGS. 23A, 23C, 23E, 23G] or AdC6-gDN [FIGS. 23B, 23F, 23D, 23H] at the indicated doses. They were boosted 4 to 8 weeks later as shown in the graph titles with the same dose of the corresponding heterologous vector. Splenocytes were tested 2 [FIGS. 23A, 23E] or 6 [FIGS. 23C, 23G] weeks or 3 [FIGS. 23B, 23F] or 4 [FIGS. 23D, 23H] months later.

FIGS. 24A-24D show recognition of N peptides by naïve T cells or T cells to control AdC or AdC-gD vectors. Splenocytes from naïve mice [FIG. 24A], mice immunized with the AdC6-N_{1-137,229-420} vector which lacks the sequence of peptide 44 [FIG. 24B], AdC6-N₁₋₂₃₃ or AdC6-N₂₃₅₋₄₂₀ [FIG. 24C], or an AdC6 vector expressing gD fused to an antigen of hepatitis B virus [FIG. 24D] were stimulated with all of the N peptides [FIGS. 24A, 24B, 24D] or the peptides nor present within the sequence expressed by the Ad vectors [FIG. 24C] for 5 hrs in vitro and then stained for surface T cell markers and intracellular IFN-g. Graphs show frequencies of CD44⁺CD8⁺ T cells and CD44⁺CD4⁺ T cells producing the cytokine over all CD44⁺ T cells within each subset. The line at 0.05% indicated that only one peptide scored positive above this limit for splenocytes of naïve mice.

FIG. 25 shows T cell epitopes defined by predication algorism and MHC class I binding to RMA-S cells. Increases of MHC class I D^{b} [left graph] or K^{b} [middle graph] upon incubation with N peptides is shown as grey bars as fold increase divided by 100 of % positive cells upon incubation with peptide over % positive cells upon incubation with medium. The rank obtained by epitope prediction is shown as black bars. R- and p-values for Pearson correlation are shown below the graphs. For MHC class II the adjusted rank is shown for each peptide [right graph].

FIGS. 26A-26D show antibody responses to different doses of the AdC-S vectors. [FIG. 26A] Experimental outline. The drops indicate bleeds. [FIG. 26B] Optical density reading for the ELISA testing sera at a 1:100 dilution from individual mice at 2 and 4 weeks after the initial immunization. Background data without sera were subtracted. Bars indicate geometric means (GM). [FIG. 26C] Antibody titers calculated by extrapolation of data obtained with serially diluted sera. Negative responses were set at 10. In FIGS. 26B and 26C lines with stars above show significant differences between the connected groups calculated by 2-way Anova with Tukey's multiple comparison test. (*) P value between 0.01-0.05, (**) p-value between 0.001-0.01, (***) p-value between 0.0001-0.001, (****) p-value <0.0001. [FIG. 26D] Antibody isotypes of sera harvested at different times from mice primed with 10¹⁰ vp of the AdC6-S_{SWE} or the AdC7-S_{SWE} vectors. For the image corrected OD value (after subtraction of background data) are stacked. Significant differences calculated by 2-way Anova with Tukey's correction are shown at the side next to the legends.

FIGS. 27A-27D show VNA responses to different dose of the AdC-S vectors. [FIGS. 27A. 27B] VNA titers to SARS-CoV-2 were tested with a S_{SWE} pseudotyped VSV vector. Titers shown for each mouse with bars representing GM represent the serum dilution that achieved ≥50% inhibition of infection. Sera were tested starting at a 1:40 dilution. Significant differences were calculated by uncorrected Fischer's LSD test: (*) P value between 0.01-0.05, (**) p-value between 0.001-0.01, (***) p-value between 0.0001-0.001, (****) p-value <0.0001. [FIG. 27A] Antibody titers after the AdC6 prime/AdC7boost; [FIG. 27B] antibody titers after the AdC7 prime/AdC7 boost. [FIG. 27C] Experimental outline for samples tested for RBD-binding antibodies in D [FIG. 27D]. RBD antibody titers are shown for individual mice as μg of antibodies/mil calculated based on a standard. As indicated by the long lines with stars to the right all samples tested after immunization had significantly higher levels of antibodies compared to sera from naïve mice. In the AdC6/AdC6 group at week 10 levels were high in the 10¹⁰ vp than the 5×10¹⁰ vp group. Significant differences calculated by 2-way Anova with Tukey's correction. [FIGS. 27A, 27B, and 27D] Negative responses were set at 10.

FIGS. 28A-28D shows factors that influence the effectiveness of a boost. [FIG. 28A] Experimental outline. [FIG. 28B] Antibody titers against S protein tested by ELISA. The design of the graphs mimic those of FIG. 26C. Significant differences within one group are shown by lines and stars above each bar graph. Differences between the groups are shown below the x-axis. Both were calculated by 2-way Anova with Tukey's correction. [FIG. 28C] VNA titers determined with a S_{SWE} pseudotyped VSV vector. Significant differences calculated uncorrected Fischer's SD test. Those between samples within one group are shown above the bar graphs, those between groups are shown below the x-axis. [FIG. 28D] VNA titers against AdC6 and AdC7 in sera harvested 8 weeks after the prime with AdC7 or AdC6. Sera of naïve mice served as controls. [FIGS. 23B-28D] Negative responses were set at 10.

FIGS. 29A-29C show antibody responses in young and old C57Bl/6 mice. [FIG. 29A] Experimental outline. [FIG. 29B] Antibody titers at 4 weeks after the boost (wk 10) by ELISA shown as in FIG. 26C. VNA titers determined by a S_{SWE} pseudotyped VSV vector are shown as in FIGS. 27A, 27B. There were no significant differences between the groups by Mann-Whitney test. Negative responses were set at 10.

FIGS. 30A-30G relate to cross-reactivity of AdC-S_{SWE} induced VNAs. Sera harvested after priming [FIG. 30A] or boosting [FIG. 30B] of ICR mice that had initially scored positive for neutralization of the VSV-S_{SWE} vectors were retested in parallel on this vector and on VSV-S_{B1.351}, VSV-S_{B1.17} and VSV-S_{B.1617.2} vectors. [FIGS. 30A, 30B] VNA titers in individual mice with bars indicating geometric means. Negative responses were set at 10. [FIG. 30C] Percentages of sera that neutralized the different variants. [FIG. 30D] Heatmap shows levels of neutralization of the variants by individual sera. [FIG. 30E] Spearman correlations between titers against the immunizing variant VSV-S_{SWE} and the other variants. [FIG. 30E] Experimental design for the results shown in FIGS. 30F and 30G. [FIG. 30F] Antibody titers that neutralized the different VSV-S vectors in individual mice tested 2 weeks after boosting. Boxes indicate geometric means. There were no significant differences between the groups as determined by multiple paired t-tests. [FIG. 30G] Heatmap shows levels of neutralization of the variants by individual sera.

FIGS. 31A-31D relate to expression and neutralization results. [FIG. 31A] Expression of the S protein by the AdC6-S_{SWE}, AdC7-S_{SWE} and AdC6-S_{SW/B1.351} vectors tested for by Western blots using Ad vectors expressing an unrelated transgene product (N or HBV) as controls. MW—molecular weight marker. [FIG. 31B] Flow blots of the indicated cells after they were stained with an FITC-labeled antibody to ACE2 (filled grey) or an isotype control (bold black line) antibody. [FIG. 31C] Results of a neutralization assay using a panel of commercially available human naïve or immune sera and the VSV_{SWE} vector on BHK21/WI-2 cells. [FIG. 31D] Fluorescent staining of VSV-S_{SWE} transduced BHK21/WI-2 cells. Immune and naïve human sera were tested at 1:100 and 1:1000 dilutions while mouse sera were tested at a 1:100 dilution. Wells that received VSV-S_{SWE} served as controls.

FIG. 32 illustrates the conservation of T cell epitopes between SARS-CoV-2 isolates.

FIG. 33A-FIG. 33I illustrate immune responses to the vaccine vectors. FIG. 33A is a schematic of the experimental design. FIG. 33B shows antibody response data for sera harvested at baseline, 2 weeks after the prime or the boost. Reactivity against SARS-CoV-2 S1/S2 proteins tested for by an ELISA. Data show area under the curves (AUC) for dilution curves generated with sera of individual mice. Control animals scored negative with adsorbance values below background and these data are shown as an AUC of 0.1. Lines show geometric means (GM). Significant differences were calculated by 2-way Anova with Tukey correction. FIG. 33C shows antibody response data for sera harvested at baseline, 2 weeks after the prime or the boost. Sera were tested by a neutralization assay using VSV-S vectors pseudotyped with the same S protein as in the vaccines. Data are shown for individual animals. Negatives are shown as a titer of 10. Lines indicate GMs. Lines with stars above show significant differences by 2-way Anova. FIG. 33D shows data for sera harvested after the boost and tested for inhibition of ACE binding to the RBD of S1 by an ELISA. Data are shown for individual mice as μg of antibody/ml calculated based on an internal standard. Negatives are shown as a titers of 1 μg/ml. Lines indicate GMs. Significant differences were calculated by Kruskal Wallis test. FIG. 33E and FIG. 33F show data for cross-reactivity against SARS-Co-V2 variants. Sera collected after the prime [FIG. 33E] or the boost [FIG. 33F] were tested for neutralization of VSV-S vectors pseudotyped with the indicated S protein variants. Group 2 [light grey circles] and group 3 [dark grey squares] are indicated by different symbols. For the statistical analysis by one-way Anova data for the two groups were combined. FIG. 33G shows data for sera harvested 2 weeks after the boost and tested for N protein-specific antibodies by an ELISA. Results are shown as AUC for individual sera from female and male hamsters. Lines show GMs. Significant differences were calculated by 2-way Anova with Tukey correction. FIG. 33H shows frequencies of CD8⁺ T cells in blood producing IFN-γ in response to N-derived peptides. Graph shows sum of responses to the 3 peptide pools. Lines show median responses. Lines with stars above show significant differences between animals that received the COVID vaccine and control animals by Kruskal Wallis test. FIG. 33I shows proportions of N-specific CD8⁺ T cell responses to the 3 peptide pools. For FIG. 33B-FIG. 33E, results for females (light circles) and males (darker squares) are indicated by the different symbols. For FIG. 33B-FIG. 33H, lines with stars above indicate significant differences: (*) p-value between 0.01-0.05, (**) p-value between 0.001-0.01., (***) p-value between 0.0001-0.001, (****) p-value <0.0001.

FIG. 34A-FIG. 34D relate to disease score and weight loss after challenge. FIG. 34A is a schematic of the experimental design. FIG. 34B shows % of female (left) and male (right) animals that scored positive for disease on the indicated days after challenge. Morning and afternoon scores were averaged. FIG. 34C shows weight loss after challenge, shown as % weigh reduction over the weight on the day of challenge. Data are shown as means±SEM. Significant differences calculated by two-way Anova with Tukey correction for day 4 and 14 data are indicated by connecting lines next to the legend. Day 4 comparisons are shown first followed by/and then day 14 comparisons. FIG. 34D shows day of maximal weight loss for Groups 1-4. Lines with stars above indicate significant differences: (*) p-value between 0.01-0.05, (**) p-value between 0.001-0.01., (***) p-value between 0.0001-0.001, (****) p-value <0.0001.

FIG. 35A-FIG. 35B relate to weight loss and lung pathology. FIG. 35A provides two graphs showing relative weights over time after challenge in female and male hamsters. FIG. 35B shows representative lung sections from animals of groups 1-4 collected on day 4 or 14 after challenge. Some of the diseased areas are highlighted by arrows.

FIG. 36A-FIG. 36F relate to viral loads and lung pathology. FIG. 36A and FIG. 36B show viral RNA [FIG. 36A] and sgRNA [FIG. 36B] loads in oral swabs collected after challenge on days 2 and 4 for all animals and on days 7 and 14 for the day 14 euthanasia group animals. FIG. 36C and FIG. 36D show lung viral RNA [FIG. 36C] and sgRNA [FIG. 36D] titers for individual hamsters of the day 4 and day 14 euthanasia groups. FIG. 36E shows sum of lung lesions for the day 4 and day 14 euthanasia group. For FIG. 36A-FIG. 36E, differences were calculated by 2-way Anova. Lines with stars above indicate significant differences: (*) p-value between 0.01-0.05, (**) p-value between 0.001-0.01., (***) p-value between 0.0001-0.001, (****) p-value <0.0001. FIG. 36F shows severity of the different types of lesions according to gender.

FIG. 37A-FIG. 37C show correlations between antibodies to N proteins and disease parameters and T cell responses. FIG. 37A is a graph showing the R-values of correlations by Spearman between N protein specific antibody responses and the indicated parameters of hamsters from groups 1, 3 and 4 for all animals. FIG. 37B is a graph showing the R-values of correlations by Spearman between N protein specific antibody responses and the indicated parameters of hamsters from groups 1, 3 and 4 for the day 4 euthanasia animals. FIG. 37C is a graph showing the R-values of correlations by Spearman between N protein specific antibody responses and the indicated parameters of hamsters from groups 1, 3 and 4 for the day 14 euthanasia animals. For FIG. 37A-FIG. 37C, significant correlations are shown by light grey bars, data that did not reach significance are shown as white bars. Level of significance is indicated by stars above the bars: (*) p-value between 0.01-0.05, (**) p-value between 0.001-0.01., (***) p-value between 0.0001-0.001, (****) p-value <0.0001.

FIG. 38 illustrates mutations within S protein that affect HLA class I epitopes. Protein sequence of S protein (SEQ ID NO: 2) is shown on top of each line; dashes (-) represent conserved amino acids; letters indicate mutations; A indicates deletions; empty spaces indicate insertion within one of the sequences; MHC class I epitopes are highlighted grey (score ≥0.9) or underlined (score 0.8-<0.9).

FIG. 39 illustrates mutations within S protein that affect HLA class II epitopes. Protein sequence of S protein (SEQ ID NO: 2) is shown on top of each line; dashes (-) represent conserved amino acids; letters indicate mutations; A indicates deletions; empty spaces indicate insertion within one of the sequences; MHC class II epitopes are highlighted grey (adjusted rank <0.5) or underlined (adjusted range ≥0.5-1).

FIG. 40 illustrates mutations within N protein that affect HLA class I epitopes. Protein sequence of N protein (SEQ ID NO: 4) is shown on top of each line; dashes (-) represent conserved amino acids; letters indicate mutations; A indicates deletions; empty spaces indicate insertion within one of the sequences; MHC class I epitopes are highlighted grey (score ≥0.9) or underlined (score 0.8-<0.9).

FIG. 41 illustrates mutations within N protein that affect HLA class II epitopes. Protein sequence of N protein (SEQ ID NO: 4) is shown on top of each line; dashes (-) represent conserved amino acids; letters indicate mutations; A indicates deletions; empty spaces indicate insertion within one of the sequences; MHC class II epitopes are highlighted grey (adjusted rank <0.5) or underlined (adjusted range ≥0.5-1).

FIG. 42A-FIG. 42D relate to T cell epitopes within SARS-CoV-2 variants. FIG. 42A is a table showing the percentage of the S and N protein sequences that are covered by putative HLA class I or II T cell epitopes. FIG. 42B is a table showing numbers of S and N protein amino acid mutations within the variants and how many of them are within T cell epitopes. FIG. 42C and FIG. 42D are tables showing different putative HLA class I epitopes (FIG. 42C) or HLA class II epitopes (FIG. 42D) and their restricting elements that are affected by mutations in the variants. The first line of each set shows to the left the AA sequence that was analyzed and to the right the mutations within this sequence. The parts below show the AA sequence that putatively binds to the HLA alleles and the left the score for the peptide sequence from the original virus (wt—wildtype) and the variants as a heatmap. The sequences analyzed for MHC class I epitopes are SEQ ID NOs: 115, 120, 124, 131, 134, 139, 142, 143, 144, 147, and 151. The putative MHC class I epitopes are SEQ ID NOs: 116-119, 121-123, 125-130, 132, 133, 135-138, 140-143, 145, 146, and 148-151. The MHC class II core epitope sequences are SEQ ID NOs: 152-160.

FIG. 43 illustrates mutations within S protein that affect linear B cell epitopes. Protein sequence of S protein (SEQ ID NO: 2) is shown on top of each line; dashes (-) represent conserved amino acids; letters indicate mutations; A indicates deletions; empty spaces indicate insertion within one of the sequences; published linear B cell epitopes are highlighted.

FIG. 44 illustrates mutations within N protein that affect linear B cell epitopes. Protein sequence of N protein (SEQ ID NO: 4) is shown on top of each line; dashes (-) represent conserved amino acids; letters indicate mutations; A indicates deletions; empty spaces indicate insertion within one of the sequences; published linear B cell epitopes are highlighted.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably +1%, and still more preferably +0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies (scFv) and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. Kappa and lambda light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

As used herein, the term “Allogeneic” refers to any material derived from a different animal of the same species.

The term “immunogenicity” as used herein, refers to the innate ability of an antigen or organism to elicit an immune response in an animal when the antigen or organism is administered to the animal. Thus, “enhancing the immunogenicity” refers to increasing the ability of an antigen or organism to elicit an immune response in an animal when the antigen or organism is administered to an animal. The increased ability of an antigen or organism to elicit an immune response can be measured by, among other things, a greater number of antibodies that bind to an antigen or organism, a greater diversity of antibodies to an antigen or organism, a greater number of T-cells specific for an antigen or organism, a greater cytotoxic or helper T-cell response to an antigen or organism, a greater expression of cytokines in response to an antigen, and the like.

As used herein, the terms “eliciting an immune response” or “immunizing” refer to the process of generating a B cell and/or a T cell response against a heterologous protein.

As used herein, by “combination therapy” is meant that a first agent is administered in conjunction with another agent. “In combination with” or “In conjunction with” refers to administration of one treatment modality in addition to another treatment modality. As such, “in combination with” refers to administration of one treatment modality before, during, or after delivery of the other treatment modality to the individual. Such combinations are considered to be part of a single treatment regimen or regime.

“Humoral immunity” or “humoral immune response” both refer to B-cell mediated immunity and are mediated by highly specific antibodies, produced and secreted by B-lymphocytes (B-cells).

“Adjuvant” refers to a substance that is capable of potentiating the immunogenicity of an antigen. Adjuvants can be one substance or a mixture of substances and function by acting directly on the immune system or by providing a slow release of an antigen. Examples of adjuvants are aluminium salts, polyanions, bacterial glycopeptides and slow release agents as Freund's incomplete adjuvant.

“Delivery vehicle” refers to a composition that helps to target the antigen to specific cells and to facilitate the effective recognition of an antigen by the immune system. The best-known delivery vehicles are liposomes, virosomes, microparticles including microspheres and nanospheres, polymeres, bacterial ghosts, bacterial polysaccharides, attenuated bacteria, virus like particles, attenuated viruses and ISCOMS.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

The term “coronavirus” as used herein refers to a member of Coronaviridae, a family of enveloped, positive-sense single-strand RNA viruses. Coronaviruses can causes disease in birds and mammals. One of the most well-studied coronaviruses is MHV, which causes epidemic infections in laboratory animals. In humans, coronaviruses typically cause respiratory infections that range in severity from the common cold to more lethal diseases such as SARS, Middle East Respiratory Syndrome (MERS), and COVID-19.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, the term “pharmaceutical composition” refers to a mixture of at least one compound useful within the invention with other chemical components, such as carriers, stabilizers, diluents, adjuvants, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two nucleic acid or amino acid molecules, such as, between two polynucleotide or polypeptide molecules. When two amino acid sequences have the same residues at the same positions; e.g., if a position in each of two polypeptide molecules is occupied by an Arginine, then they are identical at that position. The identity or extent to which two amino acid or two nucleic acid sequences have the same residues at the same positions in an alignment is often expressed as a percentage. The identity between two amino acid or two nucleic acid sequences is a direct function of the number of matching or identical positions; e.g., if half of the positions in two sequences are identical, the two sequences are 50% identical; if 90% of the positions (e.g., 9 of 10), are matched or identical, the two amino acids sequences are 90% identical.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e³ and e¹⁰⁰ indicating a closely related sequence. The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “immune response” as used herein is defined as a cellular response to an antigen that occurs when lymphocytes identify antigenic molecules as foreign and induce the formation of antibodies and/or activate lymphocytes to remove the antigen.

When “an immunologically effective amount,” “an autoimmune disease-inhibiting effective amount,” or “therapeutic amount” is indicated, the precise amount of the compositions of the present invention to be administered can be determined by a physician or researcher with consideration of individual differences in age, weight, tumor size, extent of infection or metastasis, and condition of the patient (subject).

The term “knockdown” as used herein refers to a decrease in gene expression of one or more genes.

The term “knockout” as used herein refers to the ablation of gene expression of one or more genes.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “limited toxicity” as used herein, refers to the peptides, polynucleotides, cells and/or antibodies of the invention manifesting a lack of substantially negative biological effects, anti-tumor effects, or substantially negative physiological symptoms toward a healthy cell, non-tumor cell, non-diseased cell, non-target cell or population of such cells either in vitro or in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

A “target site” or “target sequence” refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

In a remarkable achievement, SARS-CoV-2 vaccines were developed and approved for use in humans within less than a year after onset of the COVID-19 pandemic. Nevertheless, even in countries with easy access to the vaccines the pandemic has been a roller coaster ride with cycles of reductions in cases followed by rapid increases due to the emergence and spread of more transmissible variants. This constant up and down in caseloads shows how fragile society's control over SARS-CoV-2 remains. Available COVID-19 vaccines protect well against disease but largely fail to prevent infections, which allows for spread of the virus even in populations with high vaccine coverage. In addition, protection induced by mRNA vaccines, one of the most widely distributed types of COVID-19 vaccines, wanes after a few months. Resistance to COVID-19 disease can be restored at least temporarily by a booster immunization but after a third dose of an RNA vaccine, protection declines after four months, and it is currently unknown if frequent boosts with such genetic vaccines within short intervals will continue to restore antibody titers. Long-lived plasma cells can maintain antibody responses often for years after vaccination or infection. Induction of long-lived plasma cells required T cell help within germinal centers and their maintenance requires continuous survival signals in specialized niches of the bone marrow or secondary lymphatic tissues. Although COVID-19 miRNA vaccines were shown to result in germinal center formation, available evidence including the phenotypes of the vaccine-induced B cells and the rapid declines in vaccine-induced antibody titers suggest that COVID-19 mRNA vaccines induce primarily short- rather than long-lived plasma cells. This combined with the unceasing emergence of new variants that more and more escape vaccine-induced VNAs necessitates the development of second generation COVID-19 vaccines that induce sustained protection against abroad spectrum of current and future variants.

SARS-CoV-2 has thus far mainly accumulated mutations within the S protein. As other viral proteins are less variable, the present invention provides vaccines that express the N protein in addition to the S protein vaccine. The N protein is a target for non-neutralizing antibodies that might play a role in combating a SARS-CoV-2 infection. The N protein also induces CD8+ T cells that, by rapidly killing cells early after their infection, have also been implicated to contribute to protection against severe COVID-19 or to improve resistance in individuals with suboptimal VNA titers.

Although currently available vaccines avert serious COVID-19 disease and death, it is certain that additional variants with even more mutations that escape protection induced by current vaccines will evolve, new vaccines expressing additional SARS-CoV-2 antigens to augment VNA mediated protection through additional immune mechanisms are needed.

### Immunogenic Compositions

In one aspect, the current disclosure provides immunogenic compositions comprising replication-deficient adenoviral vectors that express one or more viral proteins from the SARS-CoV-2 coronavirus. These vectors are particularly useful for inducing immune responses against multiple SARS-CoV-2 proteins in subjects, thus resulting in more robust and effective immunity over vaccines that target single SARS-CoV-2 viral proteins. In some embodiments, the SARS-CoV-2 viral proteins are selected to induce immune responses that favor certain aspects of the immune response, for example antibody-mediated and T cell-mediated immunity. By inducing a multifaceted immune response, the immunogenic compositions of the invention induce more effective immune memory for longer-lasting immunity that is less susceptible to evasion by variation of any single protein (e.g. the Spike protein). Also provided are methods of stimulating an immune response in a subject, as well as methods for treating or preventing a coronavirus infection in a subject, both comprising administering an effective amount of the immunogenic compositions of the invention.

### Adenoviral Vaccine Vectors

Adenoviruses are unenveloped DNA viruses of the Adenoviridae family. Possessing double-stranded DNA (dsDNA) genomes between 34 and 48 kilobases in size and capsids between 90-100 nm in diameter, and are among the largest non-enveloped viruses. Adenoviruses are capable of infecting a wide range of mainly vertebrate hosts. In humans, over 50 distinct serotypes of adenoviruses have been isolated, and are known to cause a number of illnesses mainly of the respiratory system including the common cold, pneumonia, croup, and bronchitis.

Because of their ability to infect replicating and non-replicating cells of broad tropism without integration into the host genome, as well as their capacity to contain relatively large transgenes, adenoviruses have been widely used as viral vectors for delivering nucleic acids into target cells both in vitro and in vivo. The adenoviral genome is well-characterized and relatively easy to modify using common techniques well-known in the art. Infection with even replication-defective adenoviral vectors can induce potent humoral and cellular immune responses, a phenomenon which has led to the development of adenoviral vectors as vaccine platforms.

The global COVID pandemic caused by the SARS-CoV-2 coronavirus has led to the rapid deployment of a number of adenovirus-based vaccines for clinical use. To date, these adenoviral vaccines carry a nucleic acid encoding the SARS-CoV-2 Spike protein (S) as a single antigen. The long-term durability of the immune memory induced by these single-target adenoviral vaccines remains incompletely characterized, particularly with the increasing prevalence of spike protein variants. Thus in some embodiments, the adenoviral vectors of the current disclosure comprise one or more SARS-CoV-2 proteins, e.g., two different SARS CoV-2 proteins, such as spike (S) and nucleocapsid (N). The SARS CoV-2 nucleocapsid protein (N) comprises an N-terminal domain (NTD) and a C-terminal domain (CTD) separated by a conserved central intrinsically disordered region (IDR) containing a serine/arginine-rich region (SR) that is highly phosphorylated in infected cells. As such, the nucleocapsid protein (N) is also referred to as the nucleocapsid phosphoprotein.

In one aspect, the invention provides an immunogenic composition comprising an effective amount of a first adenoviral vector comprising a first nucleotide sequence encoding a first SARS-CoV-2 protein and a second adenoviral vector comprising a second nucleotide sequence encoding a second SARS-CoV-2 protein. In certain embodiments, the first SARS-CoV-2 protein is SARS-CoV-2 spike (S) protein and the second SARS-CoV-2 protein is SARS-CoV-2 nucleocapsid (N) protein. In this way, immune responses are directed against multiple antigens. In some embodiments, the SARS-CoV-2 spike protein comprises SEQ ID NO: 2 or SEQ ID NO: 6, and the SARS-CoV-2 nucleocapsid protein comprises SEQ ID NO: 4. In some embodiments, the first nucleotide sequence comprises SEQ ID NO: 1 or SEQ ID NO: 5, and the second nucleotide sequence comprises SEQ ID NO: 3.

In certain embodiments, the SARS-CoV-2 proteins of the adenoviral vectors of the present disclosure are selected to stimulate specific aspects of the immune response. A nonlimiting example is the use of the SARS-CoV-2 spike protein as an antigen. A key aspect of the humoral immune response is the generation of antigen-specific antibodies, including but not limited to those of the IgM and IgG types. In the course of an immune response, these antibodies act not only to bind and block spike protein interaction with its host receptor (including but not limited to angiotensin converting enzyme 2 or ACE2) thereby neutralizing their ability to enter host cells, but also to opsonize viral particles or virus-infected cells to enable their recognition and destruction by phagocytic cells or natural killer cells. In certain embodiments of the present disclosure, the adenoviral vectors comprise a nucleotide sequence encoding the SARS-CoV-2 spike protein. In certain embodiments, the SARS-CoV-2 spike protein can comprise certain modifications that enable the easy alteration of the spike protein's sequence by common laboratory techniques known by the skilled artisan in order to incorporate novel spike protein variations with mutation in the region that elicits virus-neutralizing antibodies. In certain embodiments, the modification comprises the addition of an AgeI restriction site into the spike gene. In certain embodiments, the modified S protein comprises the amino acid sequence of SEQ ID NO: 5. In certain embodiments, the modified S protein is encoded by a nucleic acid sequence set forth in SEQ ID NO: 6.

In certain embodiments, the selection of the SARS-CoV-2 target protein is selected to stimulate antigen-specific T cell responses. Ongoing characterization of successful immune responses to natural infections by coronaviruses in humans, including SARS-CoV-2 has identified the importance of a robust T cell responses. These responses provide virus-specific cytotoxic CD8+ T cells, which eliminate infected cells, but also helper CD4+ T cells which produce cytokines to support the production and affinity maturation of virus-specific antibodies.

Additionally, optimal T cell responses also result in immune memory via the development of long-lived memory T cells, which provide durable immunity to subsequent infection. This aspect of is of particular importance with respect to coronaviruses, due to the natural phenomenon of specific antibody titers fading over the course of several years following successful resolution of natural infection and possibly increasing the chances of reinfection.

In certain embodiments, the adenoviral vectors of the present disclosure comprise a nucleotide sequence encoding the SARS-CoV-2 nucleocapsid phosphoprotein (N protein). In certain embodiments, the N protein is a fusion protein that enhances its ability to stimulate T cell responses a broader range epitopes. In certain embodiments, the N protein is a fusion protein with the herpes simplex virus (HSV-1) glycoprotein D (gD). Activation of naïve CD8+ T cells induces the upregulation of a number of inhibitory receptors that act to limit the T cell response called “checkpoint inhibitor” proteins or receptors. A non-limiting example of such a receptor expressed by newly-activated T cells is BTLA or B- and T-lymphocyte attenuator, also known as CD272. BTLA interacts with HVEM or herpes virus entry mediation factor expressed by many cells including antigen presenting cells, which negatively-regulates T cell activation. While these signals are useful for limiting natural T cell responses and preventing autoimmunity and/or systemic toxicity, they can limit the efficacy of vaccines. HSV gD binds to the BTLA-binding site of HVEM, thereby inhibiting the inhibitory signals produced by this receptor and thus improving T cell activation by enhancing activation and blocking inhibitory signaling. Previous studies have demonstrated that antigens fused with HSV gD induce stronger CD8+ T cell responses, especially in aged subjects. Examples of gD fusion proteins can be found in U.S. Pat. No. 10,328,146 which is hereby incorporated by reference in its entirety. Thus, in some embodiments, the adenoviral vectors of the present disclosure combine antibody responses against the spike protein, which provide neutralizing and opsonizing antibody responses, with B and T cell responses against the nucleocapsid protein, which provides a greater cytotoxic response, and leads to greater immune memory. In certain embodiments, the nucleocapsid phosphoprotein is a fusion protein with herpes simplex virus (HSV-1) glycoprotein D (gD). In certain embodiments the nucleocapsid protein comprises an amino acid sequence set forth in SEQ ID NO: 4. In certain embodiments, the nucleocapsid sequence is encoded by a nucleic acid sequence set forth in SEQ ID NO: 3.

Selection of serotype can have a significant effect on the efficacy of the adenoviral vector-based vaccines. For example, use of adenoviral vectors based on serotypes which commonly circulate in human populations can be subject to pre-existing immunity which limits their efficacy. This is due to immune recognition and elimination of adenoviral particles before they can prime immune responses against the protein of their genetic payload. Thus, in some embodiments of the current disclosure, the adenoviral vectors are of chimpanzee origin, and therefore unlikely to have pre-existing immunity in human populations. In certain embodiments, the adenoviral vectors are of AdC6 serotype. In certain embodiments, the adenoviral vectors are of AdC7 serotype. In certain embodiments, the immunogenic composition of the present disclosure is administered according to a prime/boost schedule involving two administrations. In certain embodiments, the two administrations are of differing serotype in order to prevent immunity to the serotype-specific adenoviral proteins from reducing the efficacy of the response to the SARS-CoV-2 payload antigen proteins. In certain embodiments, the AdC6-based vaccine is administered first and the AdC7-based vaccine is administered second. In certain embodiments, the AdC7-based vaccine is administered first and the AdC6-based vaccine is administered second. It is also contemplated that other non-human adenovirus serotypes could be used in the immunogenic compositions of the invention including, but not limited to chimpanzee serotypes in addition to AdC6 and AdC7, as well as those of bovine, canine, ovine, porcine, fowl, or any combination thereof. Examples of such non-human serotypes include, but are not limited to BAd3, Cad3 OAd7, PAd3, PAd5, FAd1, FAd8, FAd9, FAd10, SAd25, or any combination thereof.

In another aspect, the invention provides a viral vector (e.g., an adenoviral vector) comprising a first nucleotide sequence encoding a first SARS-CoV-2 protein and a second nucleotide sequence encoding a second SARS-CoV-2 protein. In some embodiments, the first SARS-CoV-2 protein is SARS-CoV-2 spike (S) protein and the second SARS-CoV-2 protein is SARS-CoV-2 nucleocapsid (N) protein. In certain embodiments, the nucleocapsid protein is fused to a domain of herpes simplex virus glycoprotein D, thereby forming a fusion protein. In some embodiments, the SARS-CoV-2 spike protein comprises SEQ ID NO: 2 or SEQ ID NO: 6, and the SARS-CoV-2 nucleocapsid protein comprises SEQ ID NO: 4. In some embodiments, the first nucleotide sequence comprises SEQ ID NO: 1 or SEQ ID NO: 5, and the second nucleotide sequence comprises SEQ ID NO: 3. In certain embodiments, the viral vector is an adenoviral vector, such as a replication-defective adenoviral vector, an adenoviral vector of chimpanzee origin, or any combination thereof. In some embodiments, the adenoviral vector is serotype-6 (AdC6) or serotype-7 (AdC7).

### Methods of Use

In one aspect, the invention provides a method of stimulating an immune response in a subject, the method comprising administering to the subject an effective amount of the immunogenic composition described herein (i.e., the immunogenic composition comprising an effective amount of a first adenoviral vector comprising a first nucleotide sequence encoding a first SARS-CoV-2 protein and a second adenoviral vector comprising a second nucleotide sequence encoding a second SARS-CoV-2 protein.

In another aspect, the invention provides a method for treating or preventing a coronavirus infection in a subject, the method comprising administering to the subject an effective amount of the immunogenic composition described herein (i.e., the immunogenic composition comprising an effective amount of a first adenoviral vector comprising a first nucleotide sequence encoding a first SARS-CoV-2 protein and a second adenoviral vector comprising a second nucleotide sequence encoding a second SARS-CoV-2 protein.

In one aspect, the invention provides a method of stimulating an immune response in a subject, the method comprising administering to the subject an effective amount of the viral vector described herein (i.e., the viral vector comprising a first nucleotide sequence encoding a first SARS-CoV-2 protein and a second nucleotide sequence encoding a second SARS-CoV-2 protein).

In another aspect, the invention provides a method for treating or preventing a coronavirus infection in a subject, the method comprising administering to the subject an effective amount of the viral vector described herein (i.e., the viral vector comprising a first nucleotide sequence encoding a first SARS-CoV-2 protein and a second nucleotide sequence encoding a second SARS-CoV-2 protein).

In various embodiments, the immunogenic composition or the viral vector is delivered by a route selected from the group consisting of oral route, inhalation route, nasal route, nebulization route, intravenous injection, intraperitoneal injection, intramuscular injection, subcutaneous injection, transdermal injection, and any combination thereof. In certain embodiments, the coronavirus infection is caused by the SARS-CoV-2 virus.

In some aspects, the current disclosure also provides methods of stimulating an immune response in a subject, comprising administering to the subject a first immunogenic composition of the invention, and a second immunogenic composition of the invention. In certain embodiments, the administration of the first and second immunogenic compositions are sequential. In certain embodiments, the first immunogenic composition comprises an adenoviral vector of serotype-6 (AdC6) and the second immunogenic composition comprises an adenoviral vector of serotype-7 (AdC7). In certain embodiments, the first immunogenic composition comprises an adenoviral vector of serotype-7 (AdC7) and the second immunogenic composition comprises an adenoviral vector of serotype-6 (AdC6). In this way, administration of alternating vectors reduces the potential for immunity against the adenoviral vector to reduce the efficacy of the second immunization.

In another aspect, the current disclosure also provides a method for treating or preventing a coronavirus infection in a subject, comprising administering to the subject an effect amount of a first immunogenic composition of the current disclosure, and a second immunogenic composition of the current disclosure. In certain embodiments, the administration of the first and second immunogenic compositions of the methods of the current disclosure are sequential. In certain embodiments, the first immunogenic composition comprises an adenoviral vector of serotype-6 (AdC6) and the second immunogenic composition comprises an adenoviral vector of serotype-7 (AdC7). In certain embodiments, the first immunogenic composition comprises an adenoviral vector of serotype-7 (AdC7) and the second immunogenic composition comprises an adenoviral vector of serotype-6 (AdC6). In this way, administration of alternating vectors reduces the potential for immunity against the adenoviral vector to reduce the efficacy of the second immunization.

The methods of the current disclosure also provide for the administration of immunogenic compositions comprising nucleic acids encoding one or more SARS-CoV-2 proteins. In certain embodiments, the first immunogenic composition targets the SARS-CoV-2 spike glycoprotein and the second immunogenic composition targets the SARS-CoV-2 nucleocapsid phosphoprotein. In certain embodiments, the first immunogenic composition targets the SARS-CoV-2 nucleocapsid protein and the second immunogenic composition targets the SARS-CoV-2 spike protein. In certain embodiments, the first immunogenic composition and the second immunogenic composition both target the SARS-CoV-2 spike protein and the SARS-CoV-2 nucleocapsid protein.

In certain embodiments, the methods of the current disclosure also provide for the delivery of the one or more of the immunogenic compositions by a route selected from the group consisting of oral route, inhalation route, nasal route, nebulization route, intravenous injection, intraperitoneal injection, intramuscular injection, subcutaneous injection, transdermal injection, and any combination thereof.

In certain embodiments of the present disclosure, the coronavirus infection is caused by the SARS-CoV-2 virus.

In one aspect, the invention provides a method of stimulating an immune response in a subject, the method comprising administering to the subject: (i) an effective amount of a first adenoviral vector comprising a first nucleotide sequence encoding a first SARS-CoV-2 protein; and (ii) a second adenoviral vector comprising a second nucleotide sequence encoding a second SARS-CoV-2 protein.

In one aspect, the invention provides a method for treating or preventing a coronavirus infection in a subject, the method comprising administering to the subject: (i) an effective amount of a first adenoviral vector comprising a first nucleotide sequence encoding a first SARS-CoV-2 protein; and (ii) a second adenoviral vector comprising a second nucleotide sequence encoding a second SARS-CoV-2 protein.

In certain embodiments, the first SARS-CoV-2 protein is SARS-CoV-2 spike (S) protein and the second SARS-CoV-2 protein is SARS-CoV-2 nucleocapsid (N) protein.

In certain embodiments, the nucleocapsid protein is fused to a domain of herpes simplex virus glycoprotein D, thereby forming a fusion protein.

In certain embodiments, the SARS-CoV-2 spike protein comprises SEQ ID NO: 2 or SEQ ID NO: 6, and the SARS-CoV-2 nucleocapsid protein comprises SEQ ID NO: 4.

In certain embodiments, the first nucleotide sequence comprises SEQ ID NO: 1 or SEQ ID NO: 5, and the second nucleotide sequence comprises SEQ ID NO: 3.

In certain embodiments, the first adenoviral vector and the second adenoviral vector are replication-defective.

In certain embodiments, the first adenoviral vector is serotype-6 (AdC6) or serotype-7 (AdC7).

In certain embodiments, the second adenoviral vector is serotype-6 (AdC6) or serotype-7 (AdC7).

In certain embodiments, the first adenoviral vector and the second adenoviral vector are of the same serotype.

In certain embodiments, the first adenoviral vector and the second adenoviral vector are of different serotypes.

In certain embodiments, administration of the first adenoviral vector and administration of the second adenoviral vector each independently comprises a route selected from the group consisting of oral route, inhalation route, nasal route, nebulization route, intravenous injection, intraperitoneal injection, intramuscular injection, subcutaneous injection, transdermal injection, and any combination thereof.

In certain embodiments, the first adenoviral vector is administered prior to the second adenoviral vector.

In certain embodiments, the second adenoviral vector is administered prior to the first adenoviral vector

### Pharmaceutical Compositions

Certain embodiments of the invention are directed to prophylactically treating an individual in need thereof. As used herein, the term “prophylactic treatment” includes, but is not limited to, the administration of SARS-CoV-2 protein antigens to a subject who does not display signs or symptoms of a disease, pathology, or medical disorder, or displays only early signs or symptoms of a disease, pathology, or disorder, such that treatment is administered for the purpose of diminishing, preventing, or decreasing the risk of developing the disease, pathology, or medical disorder. A prophylactic treatment functions as a preventative treatment against a disease or disorder.

Embodiments of the present invention are directed to compositions and methods for enhancing the immune response of a subject to one or more antigens (e.g. the SARS-CoV-2 spike and nucleocapsid proteins). As used herein, the terms “subject” and “host” are intended to include living organisms such as mammals. Examples of subjects or hosts include, but are not limited to, horses, cows, sheep, pigs, goats, dogs, cats, rabbits, guinea pigs, rats, mice, gerbils, non-human primates, humans and the like, non-mammals, including, e.g., non-mammalian vertebrates, such as birds (e.g., chickens or ducks) fish or frogs (e.g., Xenopus), and a non-mammalian invertebrates, as well as transgenic species thereof. Preferably, the subject is a human.

In certain embodiments, the immunogenic compositions of the current disclosure comprise adenovirus particles made using adenovirus vectors disclosed herein. These compositions can be used to induce immunity against the encoded antigenic protein, including SARS-CoV-2 proteins. Vaccines can be formulated using standard techniques and can comprise, in addition to a replication-incompetent adenovirus vector encoding a desired protein, a pharmaceutically acceptable vehicle, such as phosphate-buffered saline (PBS) or other buffers, as well as other components such as antibacterial and antifungal agents, isotonic and absorption delaying agents, adjuvants, and the like. In some embodiments vaccine compositions are administered in combination with one or more other vaccines. Dosage units of vaccine compositions can be provided. Such dosage units typically comprise 10⁸ to 10¹¹ adenoviral particles (e.g., 10⁸, 5×10⁸, 10⁹, 5×10⁹, 10¹⁰, 5×10¹⁰, 10¹¹).

Compositions of the invention may include one or more pharmaceutically or physiologically acceptable carriers. A pharmaceutically acceptable carrier is a compound that does not itself induce the production of antibody or T cell responses harmful to the individual receiving the composition. Suitable carriers are typically large, slowly metabolized macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. Sterile pyrogen-free, phosphate-buffered, physiologic saline, is atypical carrier.

Compositions of the invention may include an antimicrobial, particularly if packages in a multiple-dose format.

Compositions of the invention may comprise a detergent e.g. a Tween (polysorbate), such as Tween 80. Detergents are generally present at low levels e.g. <0.10%.

Compositions of the invention may include sodium salts (e.g. sodium chloride) to give tonicity. A concentration of 1012 mg/ml sodium chloride is typical.

Compositions of the invention will generally include a buffer. A phosphate buffer is typical.

Compositions of the invention may include an immunogenic adjuvant. An adjuvant is a pharmacological or immunological agent that modifies the effect of other agents. Adjuvants may be added to a vaccine to boost the immune response to produce more antibodies and longer-lasting immunity, thus minimizing the dose of antigen needed. Adjuvants may also be used to enhance the efficacy of a vaccine by helping to modify the immune response to particular types of immune system cells: for example, by activating T cells instead of antibody-secreting B cells depending on the purpose of the vaccine. Immunogenic adjuvants include but are not limited to alum, MF59, AS03, Virosome, AS04, aluminum hydroxide and paraffin oil.

The regimen of administration of the compositions of the present disclosure may affect what constitutes an effective amount. For example, the adenovirus vectors of the invention may be administered to the subject (i.e. mammal) in a single dose, in several divided dosages, as well as staggered dosages may be administered sequentially, or the dose may be continuously infused, or may be a bolus injection. Sequential administrations can be separated by a period of time that can be seconds, minutes, hours, days, and weeks. Further, the dosages may be proportionally increased or decreased as indicated by the needs of the therapeutic or prophylactic situation.

Administration of the compositions of the present disclosure to a subject may be carried out using known procedures, at dosages and for periods of time effective to treat the disease in the subject. An effective amount of the composition necessary to achieve the intended result will vary and will depend on factors such as the disease to be treated or prevented, the age, sex, weight, condition, general health and prior medical history of the subject being treated, and like factors well-known in the medical arts. In particular embodiments, it is especially advantageous to formulate the composition in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the composition and the heterologous protein to be expressed, and the particular therapeutic effect to be achieved.

The administration of the immunogenic compositions of the invention may be carried out in any convenient manner known to those of skill in the art. The vaccine of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### Proteins and Nucleic Acids

The invention also provides a composition comprising one or more SARS-CoV-2 proteins including but not limited to the spike and nucleocapsid proteins. The SARS-CoV-2 proteins and their epitopes may comprise amino acid sequences that have sequence identity to the amino acid sequences disclosed in the examples. In certain embodiments, the invention provides a SARS-CoV-2 spike protein comprising SEQ ID NOs: 2 and 6. In certain embodiments, the invention provides a SARS-CoV-2 nucleocapsid protein comprising SEQ ID NO: 4. Depending on the particular sequence, the degree of sequence identity is preferably greater than 50% (e.g. 60%, 70%, 75%, 80%, 85%, 90%, 91%, 92%, 93%, 94%, 95%, 96%, 97%, 98%, 99% or more) of SEQ ID NOs: 2, 4, or 6. These proteins include homologs, orthologues, allelic variants and functional mutants. Typically, 50% identity or more between two polypeptide sequences is considered to be an indication of functional equivalence. Identity between polypeptides is preferably determined by the Smith-Waterman homology search algorithm as implemented in the MPSRCH program (Oxford Molecular), using an affine gap search with parameters gap open penalty—12 and gap extension penalty=1.

These proteins may, compared to the SARS-CoV-2 proteins of the examples, include one or more (e.g. 1, 2, 3, 4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, Substitution of single amino acids within these families does not have a major effect on the biological activity. The polypeptides may have one or more (e.g. 1, 2, 3, 4, 5, 6, 7, 8, 9, 10, etc.) single amino acid deletions relative to the SARS-CoV-2 protein sequences of the examples. The polypeptides may also include one or more (e.g. 1, 2, 3, 4, 5, 6, 7, 8, 9, 10, etc.) insertions (e.g. each of 1, 2, 3, 4 or 5 amino acids) relative to the SARS-CoV-2 sequences of the examples.

The invention further provides polypeptides comprising fusion proteins derived from the amino acid sequences of SEQ ID NOs: 2, 4, and 6. In certain embodiments, the fusion proteins comprise a SARS-CoV-2 protein fused with the herpes simplex virus (HSV-1) glycoprotein D (gD).

The SARS-CoV-2 proteins of the present disclosure may comprise at least one T-cell or B-cell epitope of the sequence. T- and B-cell epitopes can be identified empirically (e.g. using PEPSCAN 3.4 or similar methods) (Geysen, et al. (1984) PNAS USA 81:3998-4002) (Carter (1984) Methods in Molecular Biology 36:207-33), or they can be predicted (e.g. using the Jameson-Wolf antigenic index 5, matrix-based approaches (Raddrizzani, et al. (2000) Briefs in Bioinformatics 1(2):179-189), TEPITOPE (De Lalla, et al. (1999) Journal of Immunology 163:1725-1729), neural networks (Brrusic, et al. (1998) Bioinformatics 14(2):121-130), OptiMer & EpiMer (Roberts, et al. (1996) AIDS Research and Human Retroviruses 12(7):593-610), etc.).

The invention also provides nucleic acids encoding adenoviral vectors comprising one or more SARS-CoV-2 proteins including but not limited to the spike and nucleocapsid proteins. In certain embodiments, the SARS-CoV-2 spike protein is encoded by a nucleic acid comprising a polynucleotide sequence having at least 80%, 85%, 90%, 95%, 96%, 96%, 97%, 98%, 99% identity to SEQ ID NOs: 1 or 5. In certain embodiments, the SAR-CoV-2 spike protein is encoded by a nucleic acid comprising the polynucleotide sequence set forth in SEQ ID NOs: 1 or 5. In certain embodiments, the SARS-CoV-2 spike protein is encoded by a nucleic acid consisting of the polynucleotide sequence set forth in SEQ ID NOs: 1 or 5.

In certain embodiments, the SARS-CoV-2 nucleocapsid protein is encoded by a nucleic acid comprising a polynucleotide sequence having at least 80%, 85%, 90%, 95%, 96%, 96%, 97%, 98%, 99% identity to SEQ ID NO: 3. In certain embodiments, the SAR-CoV-2 nucleocapsid protein is encoded by a nucleic acid comprising the polynucleotide sequence set forth in SEQ ID NOs: 3. In certain embodiments, the SARS-CoV-2 nucleocapsid protein is encoded by a nucleic acid consisting of the polynucleotide sequence set forth in SEQ ID NOs: 3.

Tolerable variations of the nucleic acid sequences will be known to those of skill in the art. For example, in some embodiments the nucleic acid comprises a nucleotide sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the nucleotide sequences set forth in SEQ ID NOs: 1, 3, or 5.

Another aspect of the invention provides a vector comprising any one of the isolated nucleic acids disclosed herein. In certain embodiments, the vector is selected from the group consisting of a DNA vector, an RNA vector, a plasmid, a lentiviral vector, an adenoviral vector, an adeno-associated viral vector, and a retroviral vector. In certain embodiments, the vector is an expression vector.

In some embodiments, a nucleic acid of the present disclosure may be operably linked to a transcriptional control element, e.g., a promoter, and enhancer, etc. Suitable promoter and enhancer elements are known to those of skill in the art. It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

The materials and methods employed in these experiments are now described.

TABLE 1
SARS-CoV-2 Proteins.

| | SEQ | |
|---|---|---|
| Name: | ID NO: | Sequence: |
| SARS-CoV-2 | 1. | TTTGTGTTTCTGGTGCTTCTTCCATTGGTTAGCAGTCAATGCGTTAA |
| Spike | | TCTTACCACCCGCACTCAGCTGCCTCCAGCTTATACTAATAGTTTC |
| Nucleotide | | ACTCGCGGTGTGTACTATCCAGACAAAGTTTTCCGCAGCTCCGTCC |
| Sequence | | TCCATTCAACTCAAGACCTGTTCCTTCCATTTTTCAGTAATGTCACT |
| | | TGGTTTCATGCTATTCACGTTTCTGGTACCAATGGCACTAAACGCT |
| | | TCGATAACCCTGTTTTGCCTTTCAATGACGGTGTGTATTTCGCCTC |
| | | AACAGAAAAATCAAACATCATCCGCGGTTGGATTTTTGGCACTAC |
| | | CCTCGATAGTAAGACCCAGTCCCTGCTGATCGTCAATAACGCCAC |
| | | AAATGTGGTTATCAAAGTTTGTGAGTTCCAATTTTGCAACGACCCA |
| | | TTTCTTGGTGTTTACTACCATAAGAACAACAAATCATGGATGGAGT |
| | | CTGAGTTCCGCGTCTATAGCAGCGCCAACAACTGTACTTTCGAGTA |
| | | TGTCTCCCAGCCTTTCCTTATGGATCTTGAGGGCAAACAGGGGAAT |
| | | TTCAAAAACCTTCGCGAGTTTGTGTTTAAGAACATCGACGGTTATT |
| | | TTAAAATTTACAGCAAACATACTCCTATCAACTTGGTTCGCGACCT |
| | | CCCACAGGGGTTCTCCGCCCTGGAACCTCTGGTTGATTTGCCTATC |
| | | GGCATCAACATTACTCGCTTCCAGACCTTGCTCGCCCTTCACCGCT |
| | | CATACCTGACCCCTGGGGATAGTTCATCTGGCTGGACTGCTGGTGC |
| | | CGCCGCATACTACGTGGGCTATTTGCAACCTCGCACTTTTCTCCTT |
| | | AAGTATAACGAAAACGGTACTATTACCGACGCCGTTGATTGTGCA |
| | | CTCGATCCTTTGAGCGAAACCAAGTGCACTTTGAAAAGTTTTACCG |
| | | TGGAAAAAGGGATTTACCAAACATCTAATTTTCGCGTGCAGCCAA |
| | | CCGAATCCATCGTCCGCTTCCCAAATATCACTAACTTGTGTCCTTT |
| | | CGGGGAGGTTTTCAATGCTACACGCTTTGCATCTGTGTATGCTTGG |
| | | AACCGCAAGCGCATTTCAAACTGCGTTGCAGACTACTCAGTTTTGT |
| | | ACAACAGCGCTTCATTCAGCACATTTAAGTGCTATGGTGTGAGCC |
| | | CAACCAAGCTCAACGATCTTTGCTTTACCAATGTGTACGCTGACTC |
| | | TTTTGTCATCCGCGGTGATGAGGTCCGCCAGATTGCTCCAGGGCA |
| | | GACAGGTAAAATTGCCGATTACAATTACAAACTGCCTGATGATTT |
| | | TACCGGGTGCGTTATTGCCTGGAACAGCAATAATTTGGATAGCAA |
| | | AGTCGGGGGTAACTACAACTATCTTTACCGCTTGTTTCGCAAGTCT |
| | | AATCTGAAGCCATTCGAACGCGATATCAGCACTGAGATCTATCAG |
| | | GCCGGGTCAACCCCATGTAACGGGGTTGAAGGGTTCAACTGCTAT |
| | | TTCCCTCTCCAATCTTACGGTTTCCAGCCAACCAATGGGGTCGGCT |
| | | ACCAGCCATATCGCGTCGTCGTCCTCAGCTTTGAGCTGCTTCACGC |
| | | TCCTGCAACCGTCTGTGGCCCTAAGAAGTCAACAAACCTCGTGAA |
| | | AAACAAATGTGTCAACTTCAACTTCAATGGCCTCACCGGCACAGG |
| | | CGTTCTGACTGAAAGCAATAAAAAGTTCTTGCCATTTCAGCAATTC |
| | | GGTCGCGATATTGCTGATACAACAGATGCCGTTCGCGACCCACAG |
| | | ACCCTGGAAATTCTCGATATTACTCCTTGTTCTTTTGGCGGGGTGA |
| | | GCGTGATTACTCCAGGGACAAATACAAGTAACCAAGTTGCAGTGT |
| | | TGTATCAGGACGTCAACTGTACCGAGGTTCCTGTCGCAATCCACG |
| | | CCGATCAACTTACACCTACATGGCGCGTGTATAGCACTGGGAGTA |
| | | ATGTCTTCCAAACACGCGCTGGGTGCCTTATTGGGGCCGAACATG |
| | | TTAATAACTCATATGAATGCGACATCCCAATCGGCGCCGGGATCT |
| | | GCGCTAGCTACCAGACTCAGACAAACAGTCCTCGCCGCGCTCGCT |
| | | CCGTGGCAAGCCAGTCCATTATCGCCTATACTATGTCTCTCGGTGC |
| | | TGAAAACTCCGTCGCATATAGTAATAATAGCATCGCCATTCCAAC |
| | | CAATTTTACAATTAGCGTTACAACCGAGATTCTGCCTGTCTCCATG |
| | | ACCAAAACTTCCGTCGACTGCACCATGTATATCTGTGGCGACAGC |
| | | ACCGAGTGCTCCAATCTCTTGCTTCAATACGGGAGCTTCTGCACTC |
| | | AACTGAACCGCGCCTTGACTGGTATTGCAGTTGAACAAGATAAAA |
| | | ATACCCAGGAGGTCTTTGCACAAGTGAAGCAAATTTACAAAACCC |
| | | CTCCTATCAAGGACTGTGGGGGCTTTAATTTCTCACAAATCCTGCC |
| | | TGATCCATCCAAACCTAGCAAACGCTCATTCATTGAGGACCTGCTC |
| | | TTTAATAAAGTTACATTGGCTGACGCTGGTTTTATTAAACAATATG |
| | | GTGACTGTCTCGGTGACATCGCTGCCCGCGACTTGATTTGTGCACA |
| | | AAAGTTCAATGGGCTCACTGTTCTGCCACCTTTGCTCACCGATGAG |
| | | ATGATCGCACAGTATACAAGTGCCTTGCTGGCCGGCACCATCACC |
| | | TCCGGTTGGACTTTTGGTGCCGGTGCAGCTTTGCAAATCCCTTTTG |
| | | CAATGCAAATGGCTTATCGCTTTAACGGGATCGGGGTGACTCAGA |
| | | ATGTTCTGTATGAGAACCAAAAACTGATCGCCAATCAATTTAACT |
| | | CAGCCATCGGCAAGATTCAAGATTCACTGTCATCTACAGCCTCAG |
| | | CATTGGGTAAGTTGCAGGACGTCGTCAACCAAAACGCTCAGGCAC |
| | | TGAACACATTGGTCAAACAACTTAGCTCTAATTTCGGTGCCATCAG |
| | | TAGTGTGTTGAATGACATTCTTAGCCGCCTGGACAAAGTGGAAGC |
| | | CGAGGTTCAGATCGACCGCCTTATTACTGGTCGCTTGCAGTCTCTG |
| | | CAGACATATGTTACCCAACAACTCATTCGCGCCGCTGAAATTCGC |
| | | GCCAGCGCCAACCTGGCTGCCACAAAGATGTCCGAATGCGTCTTG |
| | | GGGCAAAGCAAGCGCGTTGATTTCTGCGGCAAAGGTTATCACTTG |
| | | ATGTCTTTTCCTCAATCTGCACCTCACGGGGTGGTGTTCTTGCACG |
| | | TTACTTACGTTCCAGCACAGGAGAAAAACTTCACAACAGCACCTG |
| | | CTATTTGTCATGACGGTAAGGCTCACTTCCCTCGCGAGGGTGTCTT |
| | | TGTCAGTAACGGTACTCACTGGTTTGTGACCCAGCGCAACTTTTAT |
| | | GAGCCTCAAATCATCACAACCGACAACACCTTTGTTTCCGGGAAC |
| | | TGTGATGTTGTCATTGGTATCGTCAACAACACAGTCTATGACCCTC |
| | | TTCAACCAGAACTGGACTCATTTAAGGAAGAGCTCGACAAGTATT |
| | | TTAAAAATCACACCTCTCCTGATGTGGATCTTGGGGATATCAGTGG |
| | | TATTAACGCTAGTGTGGTTAATATCCAAAAAGAAATCGATCGCCT |
| | | GAATGAAGTCGCTAAAAATCTGAATGAAAGTTTGATCGACTTGCA |
| | | AGAACTTGGGAAGTACGAACAATATATTAAATGGCCTTGGTACAT |
| | | TTGGCTCGGCTTTATCGCTGGCCTCATTGCCATCGTTATGGTTACA |
| | | ATTATGCTCTGCTGCATGACATCATGCTGCAGTTGCTTGAAAGGTT |
| | | GTTGTAGTTGTGGGTCTTGCTGTAAGTTTGATGAGGATGACTCTGA |
| | | GCCTGTTCTTAAGGGTGTGAAATTGCACTACACC |
| SARS-CoV-2 | 2. | MFVFLVLLPLVSSQCVNLTTRTQLPPAYTNSFTRGVYYPDKVFRSSVL |
| Spike Protein | | HSTQDLFLPFFSNVTWFHAIHVSGTNGTKRFDNPVLPFNDGVYFASTE |
| Sequence | | KSNIIRGWIFGTTLDSKTQSLLIVNNATNVVIKVCEFQFCNDPFLGVYY |
| | | HKNNKSWMESEFRVYSSANNCTFEYVSQPFLMDLEGKQGNFKNLRE |
| | | FVFKNIDGYFKIYSKHTPINLVRDLPQGFSALEPLVDLPIGINITRFQTL |
| | | LALHRSYLTPGDSSSGWTAGAAAYYVGYLQPRTFLLKYNENGTITDA |
| | | VDCALDPLSETKCTLKSFTVEKGIYQTSNFRVQPTESIVRFPNITNLCP |
| | | FGEVFNATRFASVYAWNRKRISNCVADYSVLYNSASFSTFKCYGVSP |
| | | TKLNDLCFTNVYADSFVIRGDEVRQIAPGQTGKIADYNYKLPDDFTG |
| | | CVIAWNSNNLDSKVGGNYNYLYRLFRKSNLKPFERDISTEIYQAGSTP |
| | | CNGVEGFNCYFPLQSYGFQPTNGVGYQPYRVVVLSFELLHAPATVCG |
| | | PKKSTNLVKNKCVNFNFNGLTGTGVLTESNKKFLPFQQFGRDIADTT |
| | | DAVRDPQTLEILDITPCSFGGVSVITPGTNTSNQVAVLYQDVNCTEVP |
| | | VAIHADQLTPTWRVYSTGSNVFQTRAGCLIGAEHVNNSYECDIPIGA |
| | | GICASYQTQTNSPRRARSVASQSIIAYTMSLGAENSVAYSNNSIAIPTN |
| | | FTISVTTEILPVSMTKTSVDCTMYICGDSTECSNLLLQYGSFCTQLNRA |
| | | LTGIAVEQDKNTQEVFAQVKQIYKTPPIKDCGGFNFSQILPDPSKPSKR |
| | | SFIEDLLFNKVTLADAGFIKQYGDCLGDIAARDLICAQKFNGLTVLPP |
| | | LLTDEMIAQYTSALLAGTITSGWTFGAGAALQIPFAMQMAYRFNGIG |
| | | VTQNVLYENQKLIANQFNSAIGKIQDSLSSTASALGKLQDVVNQNAQ |
| | | ALNTLVKQLSSNFGAISSVLNDILSRLDKVEAEVQIDRLITGRLQSLQT |
| | | YVTQQLIRAAEIRASANLAATKMSECVLGQSKRVDFCGKGYHLMSFP |
| | | QSAPHGVVFLHVTYVPAQEKNFTTAPAICHDGKAHFPREGVFVSNGT |
| | | HWFVTQRNFYEPQIITTDNTFVSGNCDVVIGIVNNTVYDPLQPELDSF |
| | | KEELDKYFKNHTSPDVDLGDISGINASVVNIQKEIDRLNEVAKNLNES |
| | | LIDLQELGKYEQYIKWPWYIWLGFIAGLIAIVMVTIMLCCMTSCCSCL |
| | | KGCCSCGSCCKFDEDDSEPVLKGVKLHYT |
| SARS-CoV-2 | 3. | TCCGATAACGGCCCACAAAATCAACGCAACGCACCTCGCATTACC |
| Nucleocapsid | | TTTGGCGGTCCTTCCGATTCTACCGGTTCAAACCAAAATGGGGAG |
| Phosphoprotein | | CGCAGTGGTGCCCGCTCAAAACAACGCCGCCCTCAAGGTCTTCCT |
| Nucleotide | | AATAATACTGCTTCATGGTTCACCGCTTTGACACAACATGGTAAA |
| Sequence | | GAGGATCTGAAATTTCCACGCGGGCAAGGGGTCCCAATTAACACC |
| | | AATTCCTCTCCAGATGACCAGATTGGGTACTATCGCCGCGCTACTC |
| | | GCCGCATCCGCGGTGGGGACGGTAAAATGAAAGATCTGTCCCCTC |
| | | GCTGGTATTTTTATTATCTTGGGACTGGGCCTGAAGCAGGTTTGCC |
| | | ATACGGGGCCAATAAAGACGGTATTATCTGGGTTGCAACAGAAGG |
| | | CGCCCTGAATACTCCAAAGGATCATATTGGGACTCGCAATCCAGC |
| | | AAATAATGCAGCTATTGTTTTGCAGCTCCCTCAGGGTACCACCCTT |
| | | CCTAAGGGTTTCTATGCAGAGGGTTCTCGCGGTGGTTCTCAGGCAT |
| | | CCTCCCGCTCATCTTCTCGCTCACGCAACAGTTCTCGCAATTCAAC |
| | | ACCAGGTTCAAATCGCGGTACCAGCCCAGCACGCATGGCTGGTAA |
| | | TGGTGGTGACGCTGCTTTGGCATTGTTGCTGCTCGATCGCCTGAAC |
| | | CAGCTGGAATCCAAGATGAGTGGGAAAGGTCAGCAACAACAGGG |
| | | TCAGACAGTCACTAAAAAGTCTGCCGCTGAGGCTTCTAAAAAGCC |
| | | ACGCCAGAAGCGCACAGCAACTAAGGCTTACAATGTGACCCAGGC |
| | | TTTTGGTCGCCGCGGTCCAGAACAGACACAAGGTAACTTTGGTGA |
| | | TCAGGAGCTCATTCGCCAAGGCACCGATTATAAACATTGGCCACA |
| | | GATTGCCCAATTCGCACCATCAGCTTCAGCCTTCTTTGGCATGTCA |
| | | CGCATCGGCATGGAGGTGACTCCTTCCGGGACATGGTTGACATAC |
| | | ACTGGTGCAATCAAACTGGACGATAAGGACCCTAATTTTAAAGAT |
| | | CAGGTCATCCTGCTTAACAAACATATTGATGCCTACAAGACTTTTC |
| | | CACCTACCGAACCTAAGAAAGACAAGAAAAAGAAGGCAGATGAG |
| | | ACTCAGGCTTTGCCTCAACGCCAAAAGAAGCAACAGACAGTTACA |
| | | CTCCTTCCAGCAGCCGATCTTGATGACTTCAGTAAACAACTGCAAC |
| | | AATCTATGAGCTCTGCAGATAGTACTCAGGCC |
| SARS-CoV-2 | 4. | MSDNGPQNQRNAPRITFGGPSDSTGSNQNGERSGARSKQRRPQGLPN |
| Nucleocapsid | | NTASWFTALTQHGKEDLKFPRGQGVPINTNSSPDDQIGYYRRATRRIR |
| Phosphoprotein | | GGDGKMKDLSPRWYFYYLGTGPEAGLPYGANKDGIIWVATEGALNT |
| Protein | | PKDHIGTRNPANNAAIVLQLPQGTTLPKGFYAEGSRGGSQASSRSSSR |
| Sequence | | SRNSSRNSTPGSNRGTSPARMAGNGGDAALALLLLDRLNQLESKMS |
| | | GKGQQQQGQTVTKKSAAEASKKPRQKRTATKAYNVTQAFGRRGPE |
| | | QTQGNFGDQELIRQGTDYKHWPQIAQFAPSASAFFGMSRIGMEVTPS |
| | | GTWLTYTGAIKLDDKDPNFKDQVILLNKHIDAYKTFPPTEPKKDKKK |
| | | KADETQALPQRQKKQQTVTLLPAADLDDFSKQLQQSMSSADSTQA |
| Spike Age I | 5. | ATGTTTGTGTTTCTGGTGCTTCTTCCATTGGTTAGCAGTCAATGCGT |
| mutant | | TAATCTTACCACCCGCACTCAGCTGCCTCCAGCTTATACTAATAGT |
| DNA sequence | | TTCACTCGCGGTGTGTACTATCCAGACAAAGTTTTCCGCAGCTCCG |
| | | TCCTCCATTCAACTCAAGACCTGTTCCTTCCATTTTTCAGTAATGTC |
| | | ACTTGGTTTCATGCTATTCACGTTTCTGGTACCAATGGCACTAAAC |
| | | GCTTCGATAACCCTGTTTTGCCTTTCAATGACGGTGTGTATTTCGC |
| | | CTCAACAGAAAAATCAAACATCATCCGCGGTTGGATTTTTGGCAC |
| | | TACCCTCGATAGTAAGACCCAGTCCCTGCTGATCGTCAATAACGC |
| | | CACAAATGTGGTTATCAAAGTTTGTGAGTTCCAATTTTGCAACGAC |
| | | CCATTTCTTGGTGTTTACTACCATAAGAACAACAAATCATGGATGG |
| | | AGTCTGAGTTCCGCGTCTATAGCAGCGCCAACAACTGTACTTTCGA |
| | | GTATGTCTCCCAGCCTTTCCTTATGGATCTTGAGGGCAAACAGGGG |
| | | AATTTCAAAAACCTTCGCGAGTTTGTGTTTAAGAACATCGACGGTT |
| | | ATTTTAAAATTTACAGCAAACATACTCCTATCAACTTGGTTCGCGA |
| | | CCTCCCACAGGGGTTCTCCGCCCTGGAACCTCTGGTTGATTTGCCT |
| | | ATCGGCATCAACATTACTCGCTTCCAGACCTTGCTCGCCCTTCACC |
| | | GCTCATACCTGACCCCTGGGGATAGTTCATCTGGCTGGACTGCTGG |
| | | TGCCGCCGCATACTACGTGGGCTATTTGCAACCTCGCACTTTTCTC |
| | | CTTAAGTATAACGAAAACGGTACTATTACCGACGCCGTTGATTGT |
| | | GCACTCGATCCTTTGAGCGAAACCAAGTGCACTTTGAAAAGTTTT |
| | | ACCGTGGAAAAAGGGATTTACCAAACATCTAATTTTCGCGTGCAG |
| | | CCAACCGAATCCATCGTCCGCTTCCCAAATATCACTAACTTGTGTC |
| | | CTTTCGGGGAGGTTTTCAATGCTACACGCTTTGCATCTGTGTATGC |
| | | TTGGAACCGCAAGCGCATTTCAAACTGCGTTGCAGACTACTCAGT |
| | | TTTGTACAACAGCGCTTCATTCAGCACATTTAAGTGCTATGGTGTG |
| | | AGCCCAACCAAGCTCAACGATCTTTGCTTTACCAATGTGTACGCTG |
| | | ACTCTTTTGTCATCCGCGGTGATGAGGTCCGCCAGATTGCTCCAGG |
| | | GCAGACAGGTAAAATTGCCGATTACAATTACAAACTGCCTGATGA |
| | | TTTTACCGGGTGCGTTATTGCCTGGAACAGCAATAATTTGGATAGC |
| | | AAAGTCGGGGGTAACTACAACTATCTTTACCGCTTGTTTCGCAAGT |
| | | CTAATCTGAAGCCATTCGAACGCGATATCAGCACTGAGATCTATC |
| | | AGGCCGGGTCAACCCCATGTAACGGGGTTGAAGGGTTCAACTGCT |
| | | ATTTCCCTCTCCAATCTTACGGTTTCCAGCCAACCAATGGGGTCGG |
| | | CTACCAGCCATATCGCGTCGTCGTCCTCAGCTTTGAGCTGCTTCAC |
| | | GCTCCTGCAACCGTCTGTGGCCCTAAGAAGTCAACAAACCTCGTG |
| | | AAAAACAAATGTGTCAACTTCAACTTCAATGGCCTCACCGGTACA |
| | | GGCGTTCTGACTGAAAGCAATAAAAAGTTCTTGCCATTTCAGCAA |
| | | TTCGGTCGCGATATTGCTGATACAACAGATGCCGTTCGCGACCCA |
| | | CAGACCCTGGAAATTCTCGATATTACTCCTTGTTCTTTTGGCGGGG |
| | | TGAGCGTGATTACTCCAGGGACAAATACAAGTAACCAAGTTGCAG |
| | | TGTTGTATCAGGACGTCAACTGTACCGAGGTTCCTGTCGCAATCCA |
| | | CGCCGATCAACTTACACCTACATGGCGCGTGTATAGCACTGGGAG |
| | | TAATGTCTTCCAAACACGCGCTGGGTGCCTTATTGGGGCCGAACA |
| | | TGTTAATAACTCATATGAATGCGACATCCCAATCGGCGCCGGGAT |
| | | CTGCGCTAGCTACCAGACTCAGACAAACAGTCCTCGCCGCGCTCG |
| | | CTCCGTGGCAAGCCAGTCCATTATCGCCTATACTATGTCTCTCGGT |
| | | GCTGAAAACTCCGTCGCATATAGTAATAATAGCATCGCCATTCCA |
| | | ACCAATTTTACAATTAGCGTTACAACCGAGATTCTGCCTGTCTCCA |
| | | TGACCAAAACTTCCGTCGACTGCACCATGTATATCTGTGGCGACA |
| | | GCACCGAGTGCTCCAATCTCTTGCTTCAATACGGGAGCTTCTGCAC |
| | | TCAACTGAACCGCGCCTTGACTGGTATTGCAGTTGAACAAGATAA |
| | | AAATACCCAGGAGGTCTTTGCACAAGTGAAGCAAATTTACAAAAC |
| | | CCCTCCTATCAAGGACTGTGGGGGCTTTAATTTCTCACAAATCCTG |
| | | CCTGATCCATCCAAACCTAGCAAACGCTCATTCATTGAGGACCTG |
| | | CTCTTTAATAAAGTTACATTGGCTGACGCTGGTTTTATTAAACAAT |
| | | ATGGTGACTGTCTCGGTGACATCGCTGCCCGCGACTTGATTTGTGC |
| | | ACAAAAGTTCAATGGGCTCACTGTTCTGCCACCTTTGCTCACCGAT |
| | | GAGATGATCGCACAGTATACAAGTGCCTTGCTGGCCGGCACCATC |
| | | ACCTCCGGTTGGACTTTTGGTGCCGGTGCAGCTTTGCAAATCCCTT |
| | | TTGCAATGCAAATGGCTTATCGCTTTAACGGGATCGGGGTGACTC |
| | | AGAATGTTCTGTATGAGAACCAAAAACTGATCGCCAATCAATTTA |
| | | ACTCAGCCATCGGCAAGATTCAAGATTCACTGTCATCTACAGCCTC |
| | | AGCATTGGGTAAGTTGCAGGACGTCGTCAACCAAAACGCTCAGGC |
| | | ACTGAACACATTGGTCAAACAACTTAGCTCTAATTTCGGTGCCATC |
| | | AGTAGTGTGTTGAATGACATTCTTAGCCGCCTGGACAAAGTGGAA |
| | | GCCGAGGTTCAGATCGACCGCCTTATTACTGGTCGCTTGCAGTCTC |
| | | TGCAGACATATGTTACCCAACAACTCATTCGCGCCGCTGAAATTC |
| | | GCGCCAGCGCCAACCTGGCTGCCACAAAGATGTCCGAATGCGTCT |
| | | TGGGGCAAAGCAAGCGCGTTGATTTCTGCGGCAAAGGTTATCACT |
| | | TGATGTCTTTTCCTCAATCTGCACCTCACGGGGTGGTGTTCTTGCA |
| | | CGTTACTTACGTTCCAGCACAGGAGAAAAACTTCACAACAGCACC |
| | | TGCTATTTGTCATGACGGTAAGGCTCACTTCCCTCGCGAGGGTGTC |
| | | TTTGTCAGTAACGGTACTCACTGGTTTGTGACCCAGCGCAACTTTT |
| | | ATGAGCCTCAAATCATCACAACCGACAACACCTTTGTTTCCGGGA |
| | | ACTGTGATGTTGTCATTGGTATCGTCAACAACACAGTCTATGACCC |
| | | TCTTCAACCAGAACTGGACTCATTTAAGGAAGAGCTCGACAAGTA |
| | | TTTTAAAAATCACACCTCTCCTGATGTGGATCTTGGGGATATCAGT |
| | | GGTATTAACGCTAGTGTGGTTAATATCCAAAAAGAAATCGATCGC |
| | | CTGAATGAAGTCGCTAAAAATCTGAATGAAAGTTTGATCGACTTG |
| | | CAAGAACTTGGGAAGTACGAACAATATATTAAATGGCCTTGGTAC |
| | | ATTTGGCTCGGCTTTATCGCTGGCCTCATTGCCATCGTTATGGTTA |
| | | CAATTATGCTCTGCTGCATGACATCATGCTGCAGTTGCTTGAAAGG |
| | | TTGTTGTAGTTGTGGGTCTTGCTGTAAGTTTGATGAGGATGACTCT |
| | | GAGCCTGTTCTTAAGGGTGTGAAATTGCACTACACCTAA |
| Spike Age I | 6. | MFVFLVLLPLVSSQCVNLTTRTQLPPAYTNSFTRGVYYPDKVFRSSVL |
| Mutant | | HSTQDLFLPFFSNVTWFHAIHVSGTNGTKRFDNPVLPFNDGVYFASTE |
| Protein | | KSNIIRGWIFGTTLDSKTQSLLIVNNATNVVIKVCEFQFCNDPFLGVYY |
| Sequence | | HKNNKSWMESEFRVYSSANNCTFEYVSQPFLMDLEGKQGNFKNLRE |
| | | FVFKNIDGYFKIYSKHTPINLVRDLPQGFSALEPLVDLPIGINITRFQTL |
| | | LALHRSYLTPGDSSSGWTAGAAAYYVGYLQPRTFLLKYNENGTITDA |
| | | VDCALDPLSETKCTLKSFTVEKGIYQTSNFRVQPTESIVRFPNITNLCP |
| | | FGEVFNATRFASVYAWNRKRISNCVADYSVLYNSASFSTFKCYGVSP |
| | | TKLNDLCFTNVYADSFVIRGDEVRQIAPGQTGKIADYNYKLPDDFTG |
| | | CVIAWNSNNLDSKVGGNYNYLYRLFRKSNLKPFERDISTEIYQAGSTP |
| | | CNGVEGFNCYFPLQSYGFQPTNGVGYQPYRVVVLSFELLHAPATVCG |
| | | PKKSTNLVKNKCVNFNFNGLTGTGVLTESNKKFLPFQQFGRDIADTT |
| | | DAVRDPQTLEILDITPCSFGGVSVITPGTNTSNQVAVLYQDVNCTEVP |
| | | VAIHADQLTPTWRVYSTGSNVFQTRAGCLIGAEHVNNSYECDIPIGA |
| | | GICASYQTQTNSPRRARSVASQSIIAYTMSLGAENSVAYSNNSIAIPTN |
| | | FTISVTTEILPVSMTKTSVDCTMYICGDSTECSNLLLQYGSFCTQLNRA |
| | | LTGIAVEQDKNTQEVFAQVKQIYKTPPIKDCGGFNFSQILPDPSKPSKR |
| | | SFIEDLLFNKVTLADAGFIKQYGDCLGDIAARDLICAQKFNGLTVLPP |
| | | LLTDEMIAQYTSALLAGTITSGWTFGAGAALQIPFAMQMAYRFNGIG |
| | | VTQNVLYENQKLIANQFNSAIGKIQDSLSSTASALGKLQDVVNQNAQ |
| | | ALNTLVKQLSSNFGAISSVLNDILSRLDKVEAEVQIDRLITGRLQSLQT |
| | | YVTQQLIRAAEIRASANLAATKMSECVLGQSKRVDFCGKGYHLMSFP |
| | | QSAPHGVVFLHVTYVPAQEKNFTTAPAICHDGKAHFPREGVFVSNGT |
| | | HWFVTQRNFYEPQIITTDNTFVSGNCDVVIGIVNNTVYDPLQPELDSF |
| | | KEELDKYFKNHTSPDVDLGDISGINASVVNIQKEIDRLNEVAKNLNES |
| | | LIDLQELGKYEQYIKWPWYIWLGFIAGLIAIVMVTIMLCCMTSCCSCL |
| | | KGCCSCGSCCKFDEDDSEPVLKGVKLHYT* |

The results of the experiments are now described.

### Example 1: Generation and Evaluation of Vectors

cDNA sequences for SARS-CoV-2 spike and nucleocapsid protein were obtained from the laboratory of Dr. Elledge, Massachusetts General Hospital, Boston, MA (sequences shown in Table 1). The spike gene contained a frame shift mutation, which was corrected.

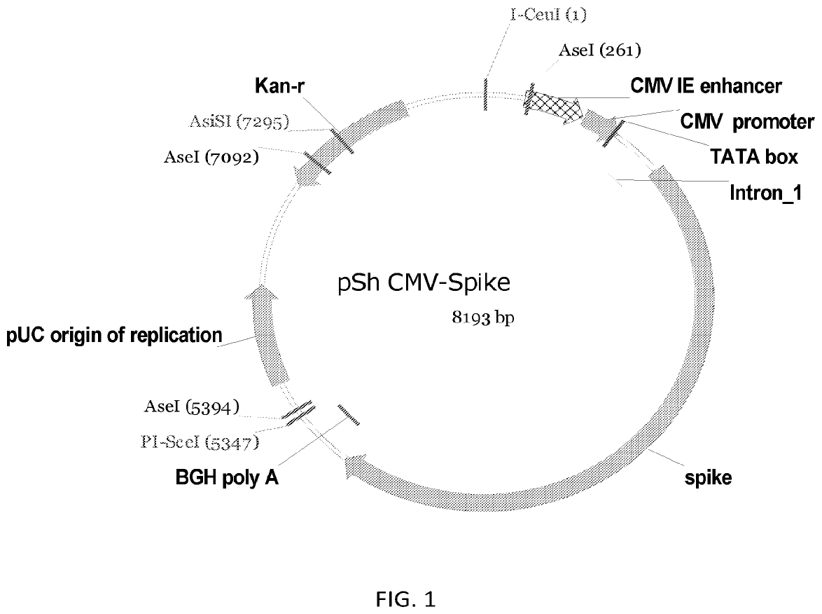

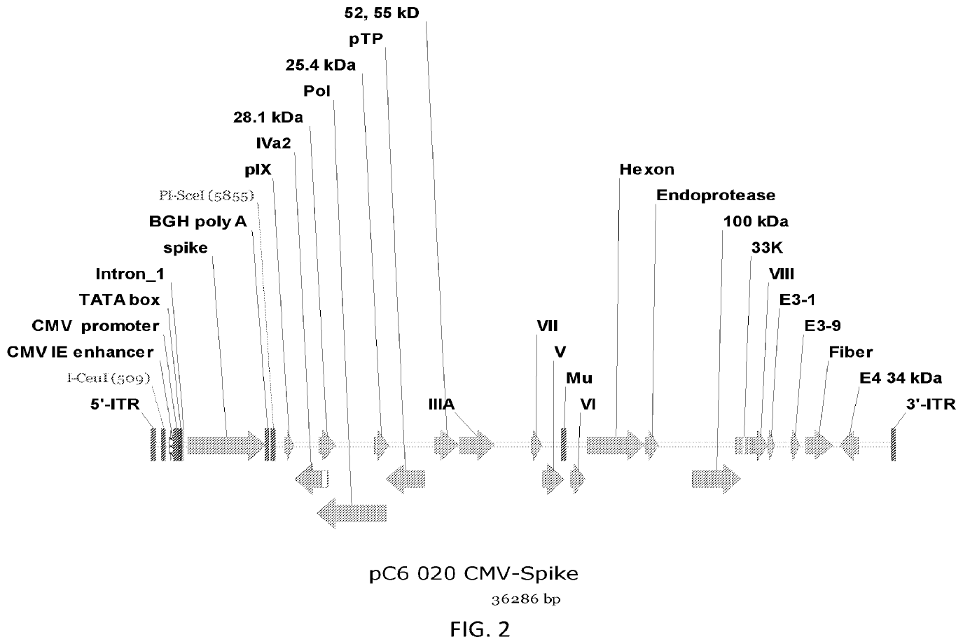

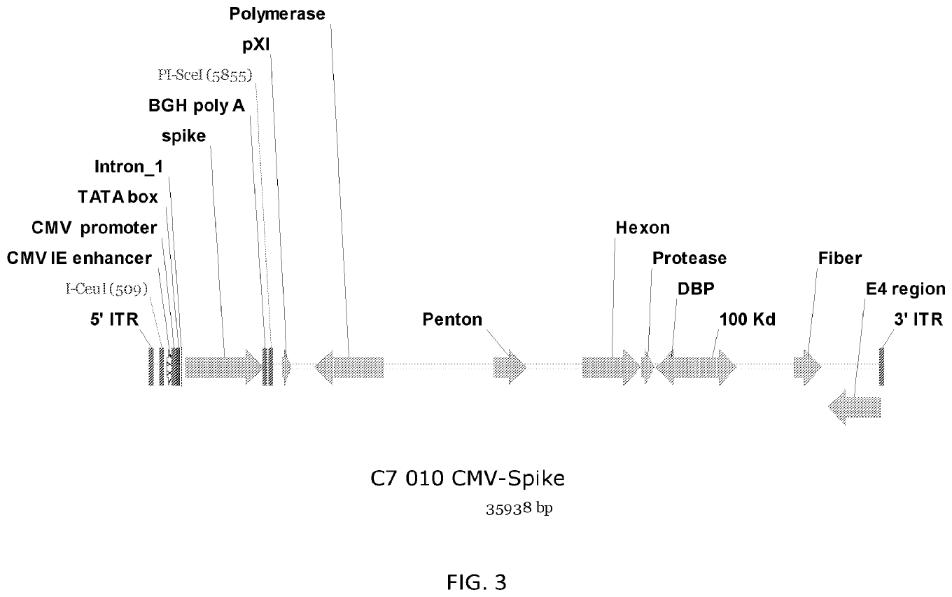

The corrected spike gene was cloned into a shuttle vector and from there into the viral molecular clones of the chimpanzee adenovirus vectors AdC6 and AdC7 (FIGS. 1-3). Correct clones were identified by restriction enzyme digest and the cloning sites were sequenced. Viral vectors named AdC6-Spike and AdC7-Spike were rescued, expanded and purified. The resulting vectors were subjected to a number a quality control studies. First, viral titers were determined by spectrophotometry. Yields for AdC6-Spike and AdC7-Spike were 1.4×10¹³ and 2.5×10¹³ respectively upon expansion in 2×10⁸ HEK293 cells. These yields are average and suitable for scale-up. Ratios of virus particles (VP) to infectious units (IU) were within an acceptable range (<600:1). Viral DNA was then purified and cut with two sets of restriction enzymes; the expected banding patterns were obtained after gel electrophoresis. Vectors were genetically stable over 12 passages.

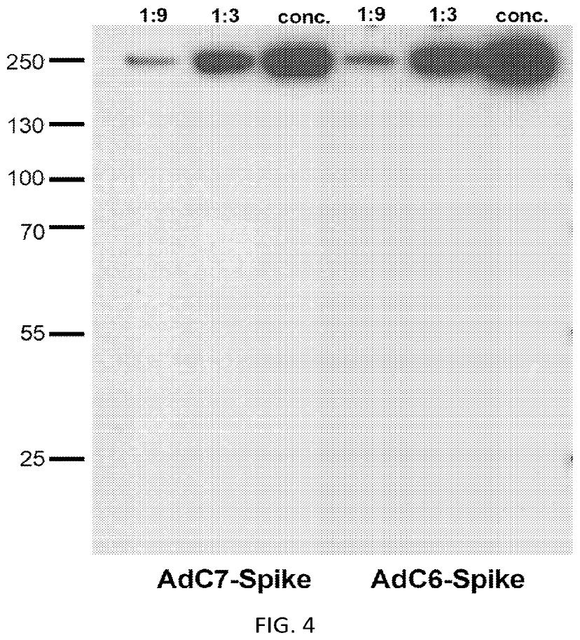

CHO cells stably transfected to express the Coxsackie adenovirus receptor (CHO-CAR) were infected with 10³ vp of the AdC6-Spike and the AdC7-Spike vector. Two days later lysates were prepared and probed by Western Blots using a polyclonal antibody rabbit antibody against the SARS-CoV-2 spike S1 subunit. A protein of the expected size was identified (FIG. 4), which was not present in a lysate from cells infected with AdC6 or AdC7 vector expressing an unrelated insert.

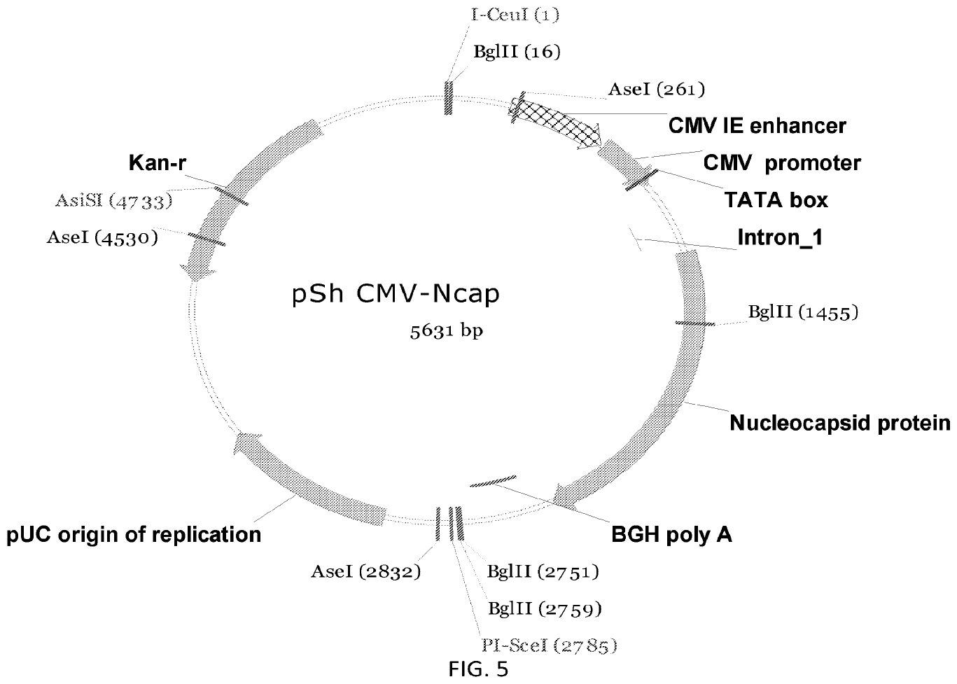

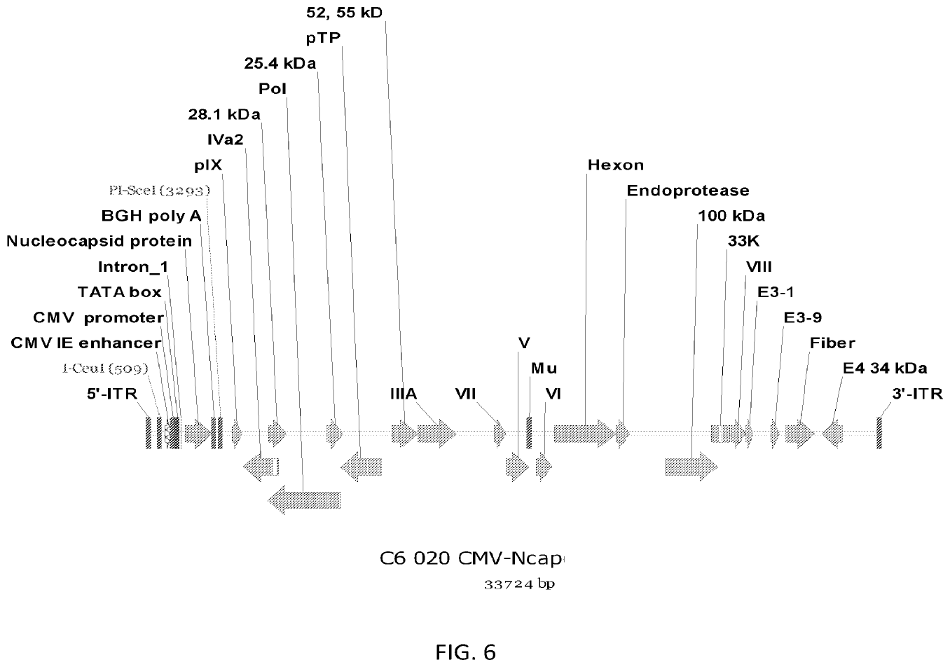

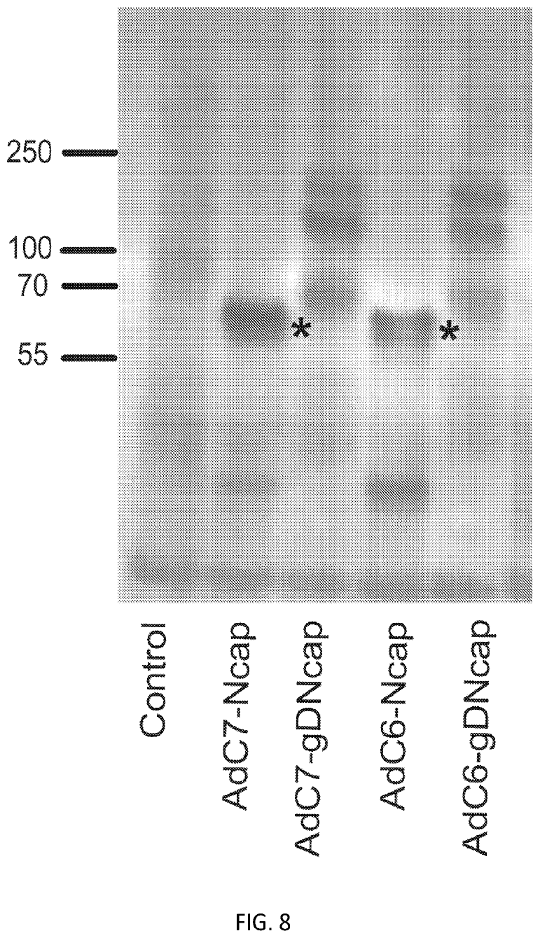

Vectors of AdC6 and AdC7 serotypes were then generated expressing the nucleocapsid protein (AdC6-NCap, AdC7-NCap, FIGS. 5-7). The nucleocapsid gene was cloned into a shuttle vector and from there into the viral molecular clones of the chimpanzee adenovirus vectors AdC6 and AdC7 (FIGS. 5-7). Correct clones were identified by restriction enzyme digest and the cloning sites were sequenced. Vectors were rescued and expanded and purified. Yields for AdC6-NCap and AdC7 NCap were 1.9×10¹³ and 2.8×10¹³ respectively upon expansion in 2×10⁸ HEK293 cells. These yields are average and suitable for scale-up. The observed VP to IU ratio was <500:1. Viral DNA was then purified and cut with two sets of restriction enzymes; the expected banding patterns were obtained after gel electrophoresis. Vectors were then passaged consecutively over 12 passages and the viral DNA was isolated from purified virus and analyzed with the same restriction enzymes. The same banding pattern was obtained indicating that vectors are genetically stable. HEK293 cells were infected with 10³ vp of the AdC6-NCap and the AdC7-NCap vector/cell. Two days later lysates were prepared and probed by Western blot using a monoclonal antibody against Ncap (Antibody 200-401-MS4). Nucleocapsid has a weight of 57 kDa. A prominent specific band was detected in cells infected with either of the 2 vectors at ˜60 kDa. This band was absent in lysates of control cells (FIG. 8).

### Example 2: Modification of the Spike Gene to Allow for Rapid Modification

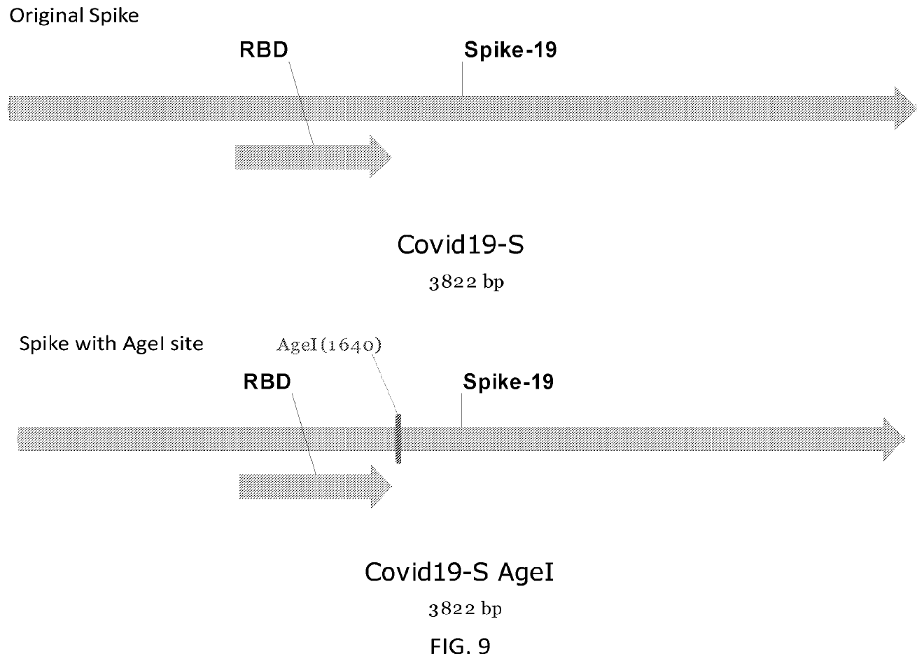

Certain modifications were then made to the spike gene to allow for rapid subsequent modification. As all viruses mutate and a number of SARS-CoV-2 mutant variants have already been isolated with changes in the receptor binding site, it is expected that eventually mutants will arise that escape the neutralizing antibodies that are induced by the currently available Spike protein-based vaccines. In order to rapidly generate vaccine vectors that express mutated versions of the spike gene, an Age 1 restriction enzyme site was inserted near the RBD of Spike, without changing the amino acid of protein (see FIG. 9 and Table 1). This change will allow rapid cloning of altered receptor binding domains to maintain vaccine efficacy with emerging variants.

### Example 3: Antibody Responses to AdC-SARS-CoV-2 Vectors in Mice

Antibody responses to Spike-expressing vectors were then evaluated by an ELISA. Plates were coated with an equal mixture of commercially available S1 and S2 proteins (Native Antigen, Oxfordshire UK) overnight. The coated plates were then blocked for 18-24 hours with a 2% bovine serum albumin (BSA) solution in phosphate buffered saline (PBS). Plates were then washed with PBS. Sera from vaccinated or control animals were diluted in 2% BSA-PBS and added to the plates, which were then incubated at room temperature for one hour. Following incubation, the plates were washed with PBS and then an alkaline phosphatase labeled goat anti-mouse IgG diluted in 2% BSA-PBS was added. Plates were incubated for 1 hour at room temperature, followed by washing with PBS. The substrate was added for ˜20 minutes and then pates were read in an ELISA reader.

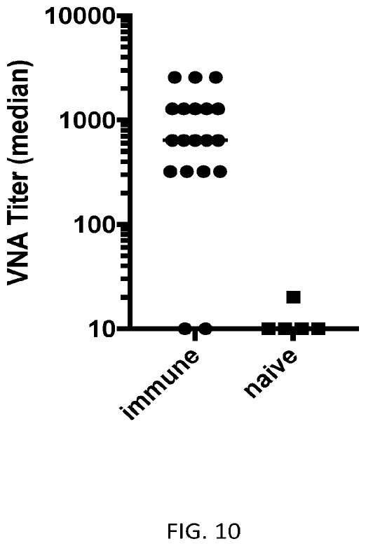

Sera were tested for virus neutralizing antibodies (VNAs) using a spike pseudotyped vesicular stomatitis virus (VSV) that expresses green fluorescent protein (GFP) upon infection of cells. BHK21/WI-2 cells, which carry the receptor for SARS-CoV-2, were diluted to 10⁶ cells/ml, 5 μl of this diluted suspension was then plated into wells of Terasaki plates, which were incubated for 18-20 hours at 37° C. in a humidified 10% C02 incubator. Sera were diluted in culture medium and incubated for 90 minutes at room temperature with a dilution of the pseudotyped VSV vector. Vector incubated with medium served as a control. 5 μl of the serum-virus mixtures were added in duplicate to the BHK21/WI-2 cells and incubated at 37° C. in a humidified C02 incubator for 20-24 hours. Plates were then read under a fluorescent microscope. The serum dilution that resulted in at least 50% reduction in GFP+ cells determined the VNA titers. The assay was validated by a commercially available pool of sera from individuals that had been infected with SARS-CoV-2 as well as from uninfected individuals (RayBiotech, Peachtree Comers, GA) (FIG. 10)

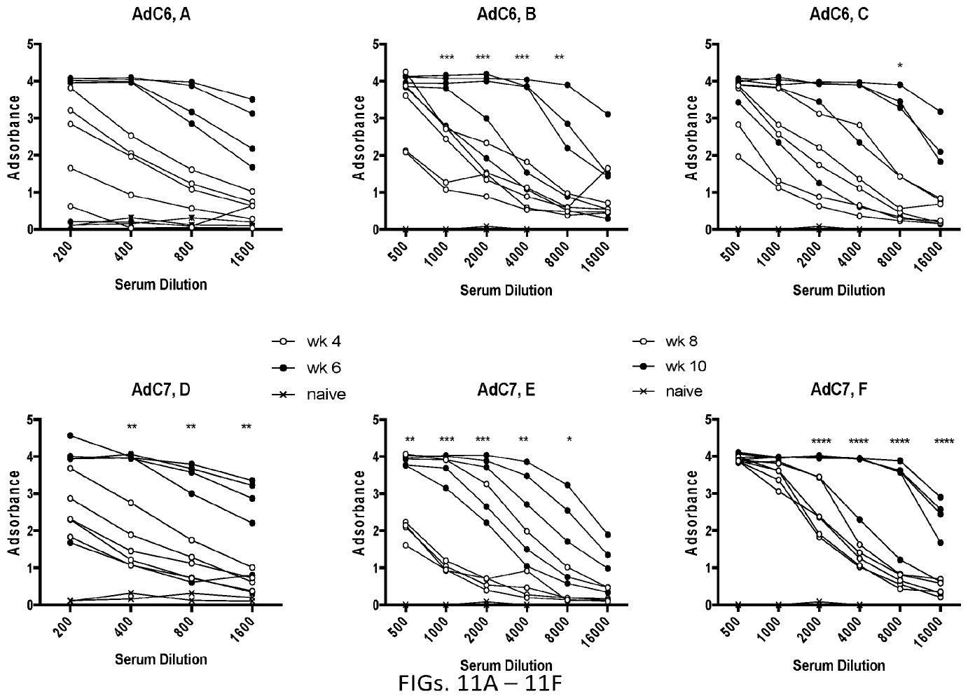

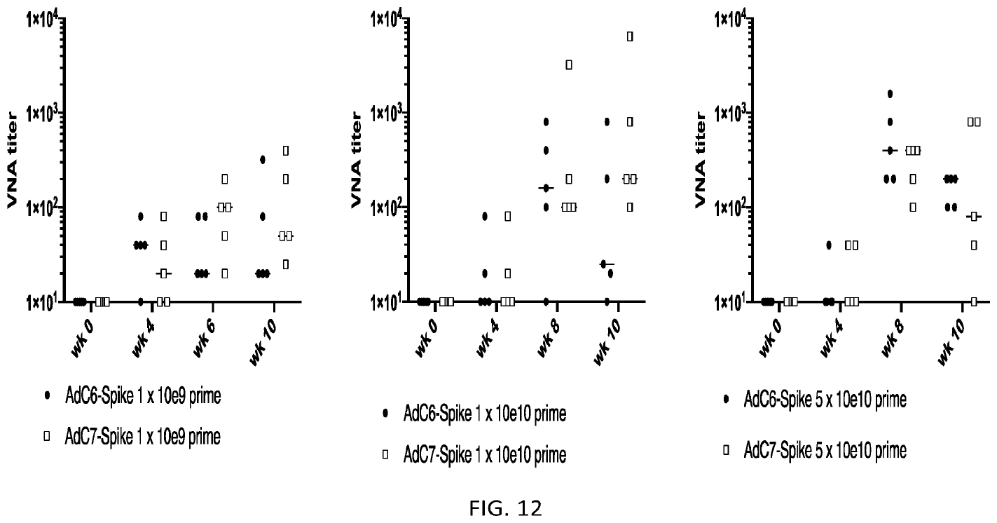

Groups of outbread mice (5 per group) were immunized with 1×10⁹, 1×10¹⁰ or 5×10¹⁰ vp of the AdC6-Spike and AdC7-Spike vectors. Mice immunized with the 1×10⁹ vp dose were boosted 4 weeks later with the heterologous vector given at the same dose. The other mice were boosted 6 weeks after the prime with the heterologous vector expressing spike given at the same dose as the vector used for the prime. The 1×10⁹ vp groups was bled just before the boost at week 4 and 2 weeks after the boost (week 6 in relation to the prime). The other groups were bled at week 6 just before the boost and then again at week 8, which is 2 weeks after the boost. Individual sera were tested at various dilutions by on S1 and S2 proteins of SARS-CoV-2. As shown in FIGS. 11A-11F both viral vectors induced in a dose-dependent manner a robust antibody response to the spike protein that increased after the boost. Further neutralization assays were then conducted with a spike-pseudotyped VSV vector. After priming some animals developed detectable titers of neutralizing antibodies. Levels of neutralizing antibodies subsequently increased after the boost. After the boost, all but 1 mouse in the 1×10¹⁰ group AdC6-Spike vector prime group developed neutralizing antibodies (FIG. 12).

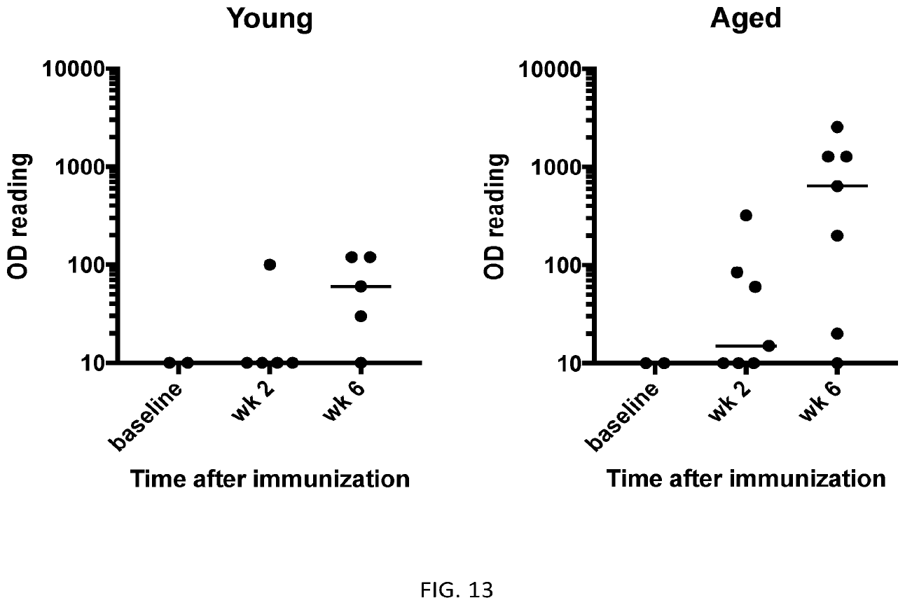

In order to assess if the spike-expressing vectors induced neutralizing antibodies a different mouse strain, i.e., C57Bl/6 mice, including mice that were older, were immunized. Older mice were 20 weeks or 9 months old. Younger mice were 6-8 weeks old at the time of priming. By 6 weeks after immunization 6 out of 7 old mice and 4 out of 5 young mice had circulating neutralizing antibodies. Antibody titers were significantly higher in the aged mice (p=0.011 by multiple t-tests) (FIG. 13).

### Example 4: T Cell Responses to the AdC-Ncap Vectors

Having demonstrated the ability of the adenoviral vectors of the invention to induce antibody responses in mice, a series of studies was then undertaken to evaluate T cell responses primed by the nucleocapsid-based vectors. Firstly, ex vivo functional studies were performed. Here, spleens were harvested from vaccinated mice. Single cell suspension was generated by mincing spleens with mesh screens in Leibovitz's L15 medium followed by passing cells through a 70 μm filter (Thermo Fisher Scientific). Red blood cells were lysed by 1×RBC lysis buffer (eBioscience, San Diego, CA).

Lymphocytes were then stimulated with various pools of peptides or individual peptides representing the SARS-CoVs nucleocapsid sequence present in the vaccines. Peptides were 15 amino acids in length and overlapped by 10 amino acids with the adjacent peptides. Individual peptides were diluted according to the manufacturer's instructions in either water, DMSO, ammonia water, formic acid or N-methyl. For stimulation ˜10⁶ lymphocytes plated in medium containing 2% fetal calf serum and Golgiplug (BD Bioscience; San Jose, CA), at 1.5 μl/ml were cultured with the peptide pools or individual peptides each present at a final concentration of 2 μg/ml for 5 h at 37° C. in a 5% CO2 incubator. Control cells were cultured without peptides.

Following stimulation cells were incubated with anti-CD8-APC (clone 53-6.7, BioLegend, San Diego CA), anti-CD4-PerCp5 (clone Gk1.5, BioLegend), anti-CD44-Alexa Flour 700 (clone IM7, BioLegend) and violet live/dead dye (Thermo Fisher Scientific) at +4° C. for 30 min in the dark. Cells were washed once with PBS and then fixed and permeabilized with Cytofix/Cytoperm (BD Biosciences, San Jose, CA) for 20 min. Following fixation, cells were incubated with an anti-INF-γ-FITC antibody (Clone, XMG1.2 BioLegend) at 4° C. for 30 min in the dark. Cells were washed and fixed in 1:3 dilution of BD Cytofix fixation buffer (BD Pharmingen, San Diego CA). They were analyzed by a BD FACS Celesta (BD Biosciences, San Jose, CA) and DiVa software. Post-acquisition analyses were performed with FlowJo (TreeStar, Ashland, OR). Data shown in graph represents % of INF-γ production by CD8+ or CD44+CD8+ cells upon peptide stimulation. Background values obtained for the same cells cultured without peptide(s) were subtracted.

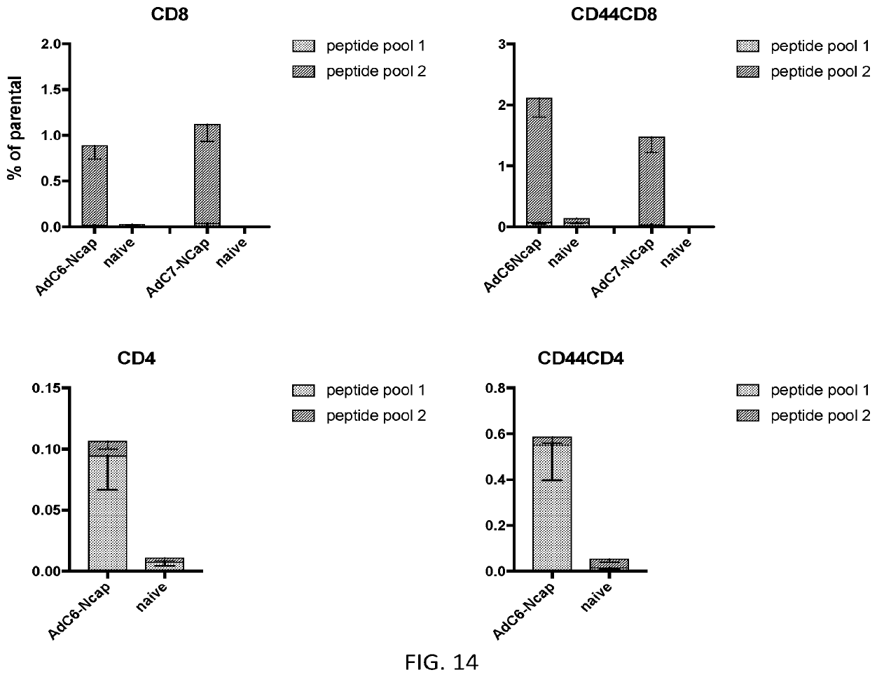

Female C57Bl/6 mice (n=5/group) were immunized i.m. with 5×10¹⁰ vp of the AdC7-Ncap vector or 2×10¹⁰ vp of the AdC6-Ncap vector. Spleens were harvested 4 (AdC7) or 2 (AdC6) weeks later and tested by intracellular cytokine staining for production of interferon (IFN)-γ in response to individual peptides (AdC7) or two peptide pools (AdC6, individual spleens, AdC7, pooled spleens) representing the sequence of the nucleocapsid. Both vectors induced robust CD8+ T cell responses mainly directed against C-terminal sequences (FIG. 13). A CD4+ T cell response was also observed that was mainly directed against the peptide pools 1 which reflects the N-terminal half of the Ncap protein. (FIG. 14).

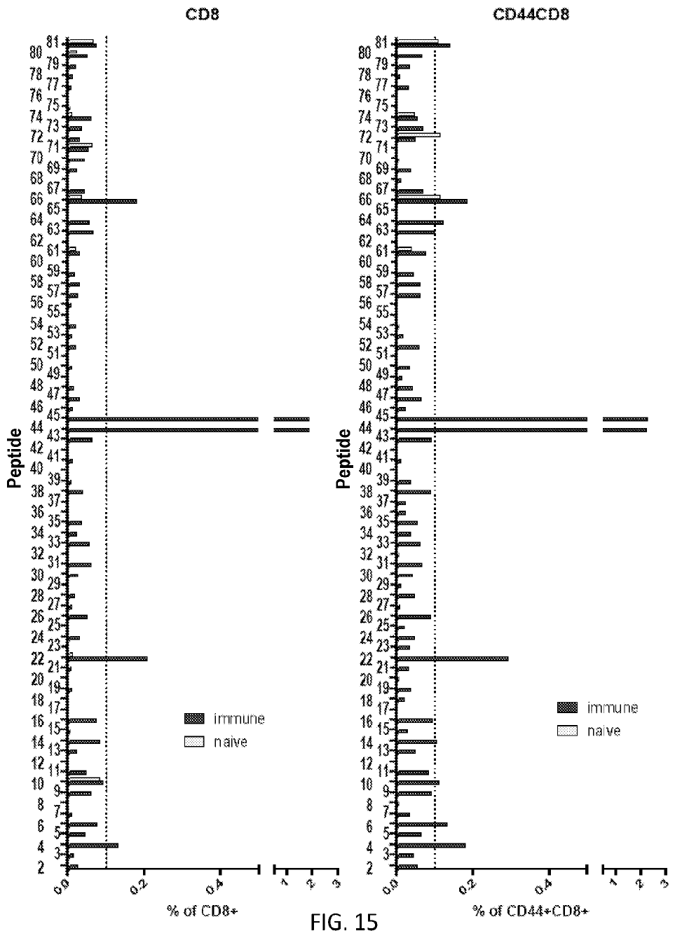

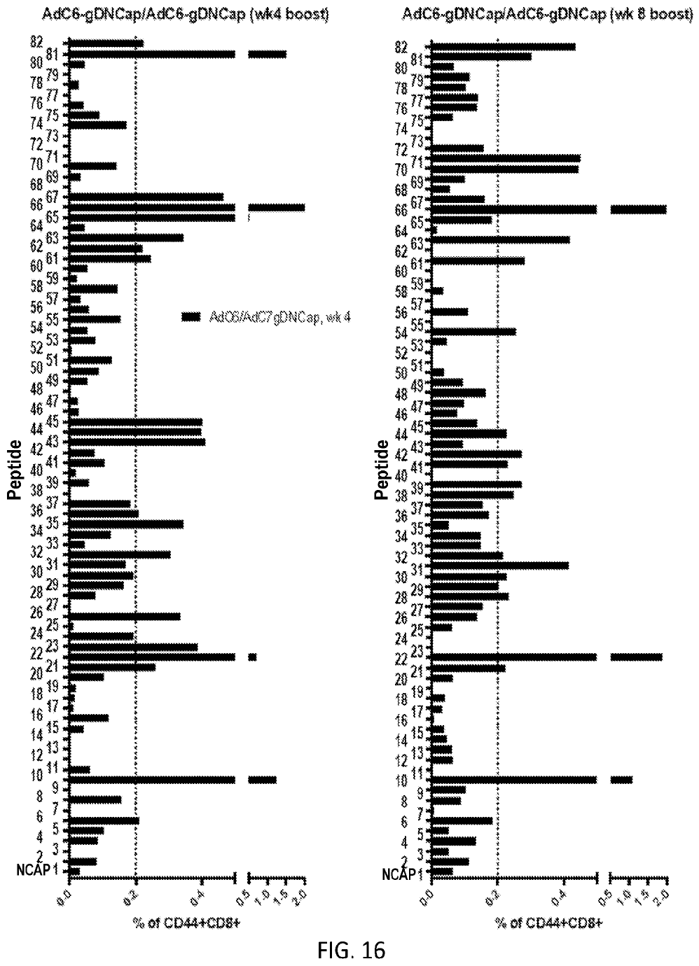

To further analyze the epitope specificity of the CD8+ T cell responses splenocytes of AdC7-NCap-immunized mice (5×10¹⁰ vp, 4 weeks earlier) were tested against individual peptides of Ncap (Table 2). As shown in FIG. 15, responses were mainly directed against peptides 22, 44, 45 and 66. In order to observe differences in the response to different NCap epitopes in a prime/boost setting, a follow-up study was conducted wherein mice were vaccinated with AdC6-gDNCap followed by a boost with AdC7-gDNCap given 4 or 8 weeks later. FIG. 16 shows frequencies of spleen-derived CD44+CD8+ cells 4 weeks after a boost delivered 4 (left) or 8 (right) weeks after the initial priming vaccination. Graphs show the IFN-7 response to individual peptides of NCap listed in Table 2. Results obtained with medium instead of peptides were subtracted.

TABLE 2
Ncap Peptide Panel.

| | SEQ ID | | | SEQ ID | |
|---|---|---|---|---|---|
| Peptide: | NO: | Sequence: | Peptide: | NO: | Sequence: |
| 1 | 7 | MSDNGPQNQRNAPRI | 42 | 48 | SPARMAGNGGDAALA |
| 2 | 8 | PQNQRNAPRITFGGP | 43 | 49 | AGNGGDAALALLLLD |
| 3 | 9 | NAPRITFGGPSDSTG | 44 | 50 | DAALALLLLDRLNQL |
| 4 | 10 | TFGGPSDSTGSNQNG | 45 | 51 | LLLLDRLNQLESKMS |
| 5 | 11 | SDSTGSNQNGERSGA | 46 | 52 | RLNQLESKMSGKGQQ |
| 6 | 12 | SNQNGERSGARSKQR | 47 | 53 | ESKMSGKGQQQQGQT |
| 7 | 13 | ERSGARSKQRRPQGL | 48 | 54 | GKGQQQQGQTVTKKS |
| 8 | 14 | RSKQRRPQGLPNNTA | 49 | 55 | QQGQTVTKKSAAEAS |
| 9 | 15 | RPQGLPNNTASWFTA | 50 | 56 | VTKKSAAEASKKPRQ |
| 10 | 16 | PNNTASWFTALTQHG | 51 | 57 | AAEASKKPRQKRTAT |
| 11 | 17 | SWFTALTQHGKEDLK | 52 | 58 | KKPRQKRTATKAYNV |
| 12 | 18 | LTQHGKEDLKFPRGQ | 53 | 59 | KRTATKAYNVTQAFG |
| 13 | 19 | KEDLKFPRGQGVPIN | 54 | 60 | KAYNVTQAFGRRGPE |
| 14 | 20 | FPRGQGVPINTNSSP | 55 | 61 | TQAFGRRGPEQTQGN |
| 15 | 21 | GVPINTNSSPDDQIG | 56 | 62 | RRGPEQTQGNFGDQE |
| 16 | 22 | TNSSPDDQIGYYRRA | 57 | 63 | QTQGNFGDQELIRQG |
| 17 | 23 | DDQIGYYRRATRRIR | 58 | 64 | FGDQELIRQGTDYKH |
| 18 | 24 | YYRRATRRIRGGDGK | 59 | 65 | LIRQGTDYKHWPQIA |
| 19 | 25 | TRRIRGGDGKMKDLS | 60 | 66 | TDYKHWPQIAQFAPS |
| 20 | 26 | GGDGKMKDLSPRWY | 61 | 67 | WPQIAQFAPSASAFF |
| | | F | | | |
| 21 | 27 | MKDLSPRWYFYYLGT | 62 | 68 | QFAPSASAFFGMSRI |
| 22 | 28 | PRWYFYYLGTGPEAG | 63 | 69 | ASAFFGMSRIGMEVT |
| 23 | 29 | YYLGTGPEAGLPYGA | 64 | 70 | GMSRIGMEVTPSGTW |
| 24 | 30 | GPEAGLPYGANKDGI | 65 | 71 | GMEVTPSGTWLTYTG |
| 25 | 31 | LPYGANKDGIIWVAT | 66 | 72 | PSGTWLTYTGAIKLD |
| 26 | 32 | NKDGIIWVATEGALN | 67 | 73 | LTYTGAIKLDDKDPN |
| 27 | 33 | IWVATEGALNTPKDH | 68 | 74 | AIKLDDKDPNFKDQV |
| 28 | 34 | EGALNTPKDHIGTRN | 69 | 75 | DKDPNFKDQVILLNK |
| 29 | 35 | TPKDHIGTRNPANNA | 70 | 76 | FKDQVILLNKHIDAY |
| 30 | 36 | IGTRNPANNAAIVLQ | 71 | 77 | ILLNKHIDAYKTFPP |
| 31 | 37 | PANNAAIVLQLPQGT | 72 | 78 | HIDAYKTFPPTEPKK |
| 32 | 38 | AIVLQLPQGTTLPKG | 73 | 79 | KTFPPTEPKKDKKKK |
| 33 | 39 | LPQGTTLPKGFYAEG | 74 | 80 | TEPKKDKKKKADETQ |
| 34 | 40 | TLPKGFYAEGSRGGS | 75 | 81 | DKKKKADETQALPQR |
| 35 | 41 | FYAEGSRGGSQASSR | 76 | 82 | ADETQALPQRQKKQQ |
| 36 | 42 | SRGGSQASSRSSSRS | 77 | 83 | ALPQRQKKQQTVTLL |
| 37 | 43 | QASSRSSSRSRNSSR | 78 | 84 | QKKQQTVTLLPAADL |
| 38 | 44 | SSSRSRNSSRNSTPG | 79 | 85 | TVTLLPAADLDDFSK |
| 39 | 45 | RNSSRNSTPGSNRGT | 80 | 86 | PAADLDDFSKDDFSK |
| 40 | 46 | NSTPGSNRGTSPARM | 81 | 87 | DDFSKDDFSKMSSAD |
| 41 | 47 | SNRGTSPARMAGNGG | 82 | 88 | QLQQSMSSADSTQA |

### Example 5: T Cell Responses to Adenoviral Vectors Expressing the SARS-CoV-2 Nucleoprotein

Here, vaccines based on two replication-defective chimpanzee adenovirus (AdC) vectors from different serotypes expressing SARS-CoV-2 N either in its wild-type form or fused into herpes simplex virus glycoprotein D (gD), which inhibits an early checkpoint that blunts T cell activation, are described. The vaccines induce potent and sustained N-specific CD8⁺ T cell responses that are broadened upon inclusion of gD.

Depending on the vaccine regimen booster immunizations increase magnitude and breadth of T cell responses. Epitopes that are recognized by the vaccine-induced T cells are highly conserved among global SARS-CoV-2 isolates indicating that addition of N as a T cell-inducing component to COVID-19 vaccines may lessen the risk of loss of vaccine-induced protection due SARS-CoV-2 variants.

### Material and Methods

### Cell Lines

HEK-293 cells and CAR-transduced CHO cells were maintained in Dulbecco's Modified Eagles medium (DMEM) supplemented with 10% fetal bovine serum (FBS) and antibiotics. RMA-S cells were grown in DMEM supplemented with 2% FBS and 0.05 M of 2-mercaptoethanol.

### Construction and Quality Control of the AdC Vectors.

The cDNA sequence for N of SARS-CoV-2 within the paT7-N plasmid is shown in Table 3 (SEQ ID NO: 4):

Amino acid sequence of SARS-CoV-2 N
1* 2 3 4 5 6 7 8 9 10
MSDNGPQNQRNAPRITFGGPSDSTGSNQNGERSGARSKQRRPQGLPNNTA
11 12 13 14 15 16 17 18 19 20
SWFTALTQHGKEDLKFPRGQGVPINTNSSPDDQIGYYRRATRRIRGGDGK
21 22 23 24 25 26 27 28 29 30
MKDLSPRWYFYYLGTGPEAGLPYGANKDGIIWVATEGALNTPKDHIGTRN
31 32 33 34 35 36 37 38 38 40
PANNAAIVLQLPQGTTLPKGFYAEGSRGGSQASSRSSGRSRNSSRNSTPG
41 42 43 44 45 46 47 48 49 50
SNRGTSPARMAGNGGDAALALLLLDRLNQLESKMSGKGQQQQGQTVTKKS
51 52 53 54 55 56 57 58 59 60
AASASKKPRQKRTATKAYNVTQAFGRRGPEQTQGNFGDQELIRQGTDYKH
61 62 63 64 65 66 67 68 69 70
WPQIAQFAPSASAFFGMSRIGMEVTPSGTWLTYTGAIKLDDKDPNFKDQV
71 72 73 74 75 76 77 78 79 80
ILLNKHIDAYKTFPPTEPKKDKKKKADETQALFQRQKKQQTVTLLPAADL
81 82
DDFSKQLQQSMSSADSTQA
*Numbers above indicate the 1st amino acids of each 15mer peptide

The paT7-N plasmid was digested with ApaI and NotI, and the N sequence was cloned into the backbone plasmid pShCMV-eGFP, which had been digested by ScaI and NotI, thus replacing eGFP and resulting in pShCMV-N. N₁₋₂₃₃ which expresses the N-terminal 233 amino acids was generated from pShCMV-N upon digestion with PvuII and ScaI followed by self-ligation; N₂₃₅₋₄₂₀, which expresses the 214 amino acid long C-terminal part of the protein was formed by cutting pShCMV-N with AgeI and PvuII. N_{1-137,229-420} which lacks 92 amino acids in the central part of N was produced by cutting pShCMV-N with SfoI and PvuII followed by self-ligation.

To generate the gDN fusion gene, the pShCMV-gD was used as the backbone plasmid. PCR cloning strategy was used to remove start and stop codons of the N gene with the forward primer: 5′-GCGGGCCCTCCGATAACGGCCCACAAAATC-3′ (SEQ ID NO: 89) and the reverse primer: 5′-GCGGGCCCGGCCTGAGTACTATCTGCAG-3′ (SEQ ID NO: 90); pShCMV-gD and the N gene PCR product were digested by ApaI enzyme and then ligated resulting in pSh-gDN plasmid.

The expression cassettes, which carry the N or gDN genes under the control of the cytomegalovirus (CMV) immediate early (IE) enhancer and the CMV promoter followed by an intron to improve expression and terminated by the bovine growth hormone polyadenylation (BGH polyA) signal, were excised from the pSh-CMV plasmids and cloned into the viral molecular clones of AdC6 and AdC7 using the rare restriction enzyme sites for I-CeuI and PI-SceI. Each cloning step was verified by restriction enzyme digest and sequencing of the insertion sites. The recombinant viral molecular clones were linearized and transfected into HEK-293 cells. Once viral plaques developed, cells were harvested, virus was released and expanded over several rounds on HEK-293 cells. Upon purification by CsCl gradient centrifugation AdC vectors were formulated in 2.5% Glycerol/25 mM NaCl/20 mM TRIS buffer, pH 8.0. Virus particle content was determined by spectrophotometry and infectious units were measured by a nested reverse transcription (RT)-polymerase chain reaction (PCR) conducted with RNA of HEK-293 cells that had been infected for 7 days with serial dilutions of the vectors. Results showed that both yields and virus particle to infectious unit ratios were within acceptable ranges. Genetic integrity of the AdC vector genome was determined by restriction enzyme digest of purified viral DNA followed by gel electrophoresis. Genetic stability was establish using the same procedure with viruses that had been passaged 12 times sequentially on HEK-293 cells. The AdC vectors passed the quality control assays.

### Protein Expression

The AdC vectors were tested for protein expression upon transfection of HEK-293 cells or CHO cells stably transfected to express CAR. Briefly, 1×10⁶ cells/flask were infected for 48 hours with ˜1000 vp of the AdC6 or AdC7 vectors expressing N or gDN. Negative control cells were transfected with an AdC vector expressing an unrelated protein. Cells were collected and lysed in RIPA buffer supplemented with a 1% μl protease inhibitor (Santa Cruz Biotechnology Inc., Dallas, TX). The lysate was stored at −80° C. until further use. 15 ul of protein sample was resolved on 12% SDS-PAGE and transferred to a polyviny-lidene difluoride (PVDF) membrane (Merck Millipore, Burlington, MA). The membrane was blocked in 5% powder milk (blocking buffer) overnight at 4° C. The primary antibody to gD (clone PA1-30233, Invitrogen, Carlsbad, CA) diluted to 1:1000 in blocking buffer was added for 1 hr at room temperature. Anti-SARS-CoV-2 N (rabbit) antibody (Rockland; Cat #200-401-MS4) was used at dilution of 1:1000 followed by HRP-conjugated goat anti-rabbit IgG (Abcam; Cat #ab6721) at a dilution of 1:10000 for 1 hr. β-Actin protein was detected by β-actin mouse monoclonal IgG antibody (Santa Cruz Biotechnology; Cat #Sc-47778) at dilution of 1:1000, followed by HRP-conjugated Goat anti-mouse IgG (Sigma; Cat #SAB3701047) at dilution of 1:5000 for 1 hr.

Membranes were washed with 1×TBS-T prior to incubating with HRP-conjugated goat anti-rabbit secondary IgG (ab6721, Abcam, Cambridge UK) for 1 hr at room temperature. Membranes were washed 3 times with 1×TBS-T. The developing agent Super Signal West Pico Chemiluminescent (Thermo Fisher Scientific, Waltham, MA) was added. Membranes were shaken in the dark for 5 min, dried and developed.

### Liquid Chromatography Tandem Mass Spectrometry

AdC-gDN-infected cells (1000 vp/cell for 24 hours) were lysed, and sample proteins were run through a 12% SDS-PAGE gel. Gel regions (55 to 100 kDa) were excised, reduced with TCEP, alkylated with iodoacetamide, and digested in-gel with trypsin. Liquid chromatography tandem mass spectrometry (LC-MS/MS) analysis was performed by the Proteomics and Metabolomics Facility at The Wistar Institute using a Q Exactive HF mass spectrometer (ThermoFisher Scientific) coupled with an UltiMate 3000 nano UPLC system (Thermo Scientific). Samples were injected onto a PepMap100 trap column (0.3×5 mm packed with 5 μm C18 resin; Thermo Scientific), and tryptic peptides were separated by reversed phase HPLC on a BEH C18 nanocapillary analytical column (75 μm i.d.×25 cm, 1.7 μm particle size; Waters) using a 2-hour gradient formed by solvent A (0.1% formic acid in water) and solvent B (0.1% formic acid in acetonitrile).

Eluted peptides were analyzed by the mass spectrometer set to repetitively scan m/z from 400 to 1500 in positive ion mode. The full MS scan was collected at 60,000 resolution followed by data-dependent MS/MS scans at 15,000 resolution on the 20 most abundant ions exceeding a minimum threshold of 20,000. Peptide match was set as preferred, exclude isotopes option and charge-state screening were enabled to reject singly and unassigned charged ions.

Peptide sequences were identified using MaxQuant 1.6.15.0 (Ref. PMID 19029910). MS/MS spectra were searched against a UniProt human protein database (Oct. 10, 2019) using full tryptic specificity with up to two missed cleavages, static carboxamidomethylation of Cys, and variable Met oxidation, Asn deamidation and protein N-terminal acetylation. Consensus identification lists were generated with false discovery rates of 1% at protein, and peptide levels.

### Mice

Female 6-week-old C57BL/6 mice were purchased from Jackson Laboratory (Bar Harbor, ME, USA). Mice were housed at the Animal Facility of the Wistar Institute and treated according to approved protocols. Unless stated otherwise experiments were conducted with groups of 5 mice 2 or 3 times.

### Vaccination and Infection of Mice

AdC6 or AdC7 vectors were diluted in sterile saline. A total volume of 200 μl containing the indicated numbers of vp was injected intramuscularly into the left hindleg of mice.

### Preparation of Splenocytes

Spleens were harvested from mice. Single cell suspension was generated by mincing spleens with mesh screens in Leibovitz's L15 medium followed by passing cells through a 70 μm filter (Thermo Fisher Scientific). Red blood cells were lysed by 1×RBC lysis buffer (eBioscience, San Diego, CA).

### In Vitro Stimulation of Lymphocytes

Lymphocytes were stimulated with pools of peptides or individual peptides (GenScript USA Inc; purity 90%). Peptides were 15 amino acids in length and overlapped by 10 amino acids with the adjacent peptides. Individual peptides were diluted according to the manufacturer's instructions in either water, DMSO, ammonia water, formic acid or N-methyl. For stimulation ˜10⁶ lymphocytes plated in medium containing 2% FBS and Golgiplug (BD Bioscience; San Jose, CA), at 1.5 μl/ml were cultured with the peptide pools or individual peptides each present at a final concentration of 2 μg/ml for 5 hr at 37° C. in a 5% CO₂ incubator. Control cells were cultured without peptides.

### Intracellular Cytokine Staining (ICS) and Analyses by Flow Cytometry

Following stimulation cells were incubated with anti-CD8-APC (clone 53-6.7, BioLegend, San Diego CA), anti-CD4-PerCp5 (clone Gk1.5, BioLegend), anti-CD44-Alexa Flour 700 (clone IM7, BioLegend) and violet live/dead dye (Thermo Fisher Scientific) at 4° C. for 30 min in the dark. Cells were washed once with PBS and then fixed and permeabilized with Cytofix/Cytoperm (BD Biosciences, San Jose, CA) for 20 min. Following fixation, cells were incubated with an anti-INF-γ-FITC antibody (Clone, XMG1.2 BioLegend) at 4° C. for 30 min in the dark. Cells were washed and fixed in 1:3 dilution of BD Cytofix fixation buffer (BD Pharmingen, San Diego CA). They were analyzed by a BD FACS Celesta (BD Biosciences, San Jose. CA) and DiVa software. Post-acquisition analyses were performed with FlowJo (TreeStar, Ashland, OR). Data shown in graph represents % of INF-γ production by CD8⁺ or CD44⁺CD8⁺ cells upon peptide stimulation. Background values obtained for the same cells cultured without peptide(s) were subtracted.

### RMA-S Assay

5×10⁵/well RMA-S cells were seeded in DMEM supplemented with 2% FBS and 0.05 M of 2-mercaptoethanol and incubated overnight at 37° C. 100 ul of individual N peptides were added at 5×10⁻⁵ M into each well. Additional cells were incubated with the peptide pools (positive control) and no peptides (negative control). Cells were incubated with peptides for 6 hrs at 37° C. Then media was discarded and replaced an H-2K^{b} antibody conjugated to PE (BD Pharmingen; Cat #553570) and an H-2D^{b} antibody conjugated to FITC (BD Pharmingen; Cat #553573) at a dilution of 1:100 in cell staining buffer (Biolegend; Cat #420201). Live/Dead fixable violet dead cell stain (Invitrogen by Thermo Fisher Scientific; Lot #2256722) was used at a dilution of 1:400 to determine cell viability; 50 ul/well of this staining dilution was added, and cells were incubated for 1 hr at 4° C. Cells were washed twice with 150 ul of cell staining buffer (×2), followed by resuspension in 1× stabilizing fixative (BD Stabilizing Fixative; REF #338036). The level of H-2K^{b} and H2-D^{b} binding to each individual peptide were detected by flow cytometry.

### Analysis of Conservation of Sequences Between Different Unique SARS-CoV-2 Isolates

For each geographic region data were acquired from the database of the National Center for Biotechnology Information using the keywords for ‘Severe acute respiratory syndrome coronavirus 2 (SARS-CoV-2)’, ‘taxid:2697049’ for virus and ‘nucleocapsid phosphoprotein’ for protein. The analyses included data available by May 18, 2021. Using Excel, duplicates and incomplete sequences were removed, and the remaining sequences were screened for presence of 15mer peptide sequences.

### Results

### AdC Vectors

Seven AdC vectors expressing SARS-CoV-2 N were constructed. They included AdC6 and AdC7 vectors expressing the full-length protein in its wild-type form (AdC6-N, AdC7-N) or fused into HSV-1 gD (AdC6-gDN, AdC7-gDN). Three truncated versions of N were expressed by AdC6: N₁₋₂₃₃ which expresses the N-terminal 233 amino acids, N₂₃₅₋₄₂₀ which expresses the 214 amino acid long C-terminal part of the protein and N_{1-137,229-420}, which lacks 92 amino acids in the central part of N.

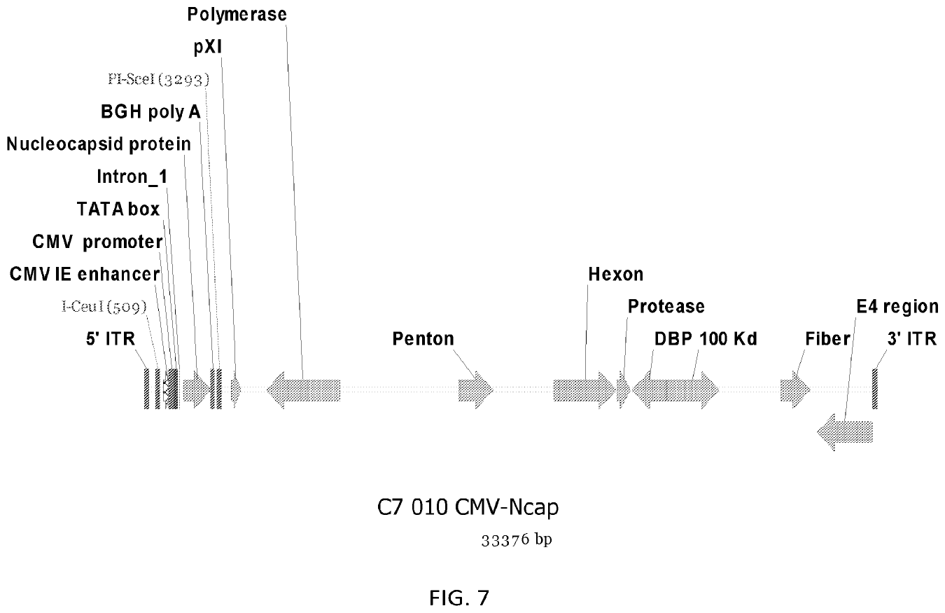

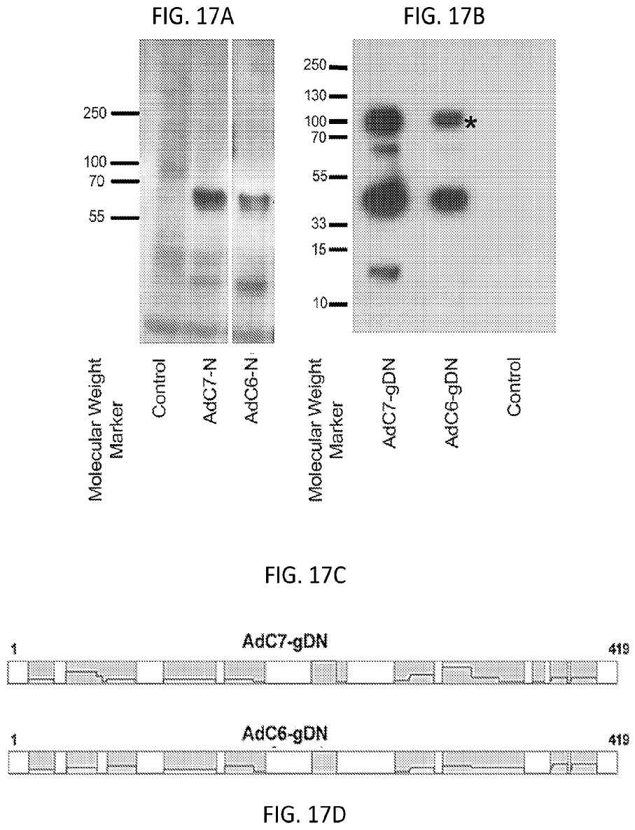

Protein expression by vectors expressing the full-length wild-type N was confirmed by Western Blot analysis using an N-specific antibody or, for vectors carrying N within gD, an antibody to gD (FIGS. 17A, 17B). Results with the latter antibody were confirmed by liquid chromatography with tandem mass spectrometry, which revealed the presence of multiple N-derived polypeptides spanning the entire sequence of the protein (FIGS. 7C, 17D).

### T Cell Responses to N Expressed by the AdC Vectors

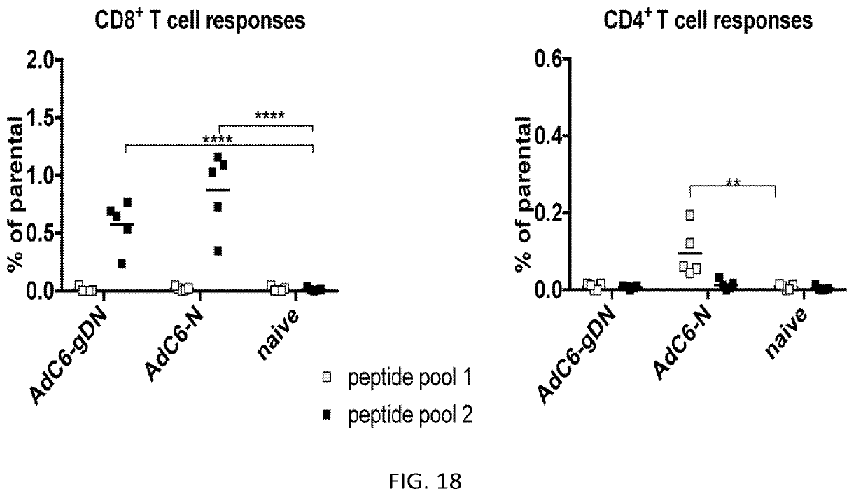

Initially, to confirm immunogenicity of N as presented by the AdC vectors, groups of 5 C57Bl/6 mice were immunized with 2×10¹⁰ vp of the AdC6-N or AdC6-gDN vectors. Naïve mice served as controls. Splenocytes were tested for responses to two pools, which each contained 41 peptides spanning the sequence of N. Peptides were 15 amino acids in length and overlapped by 10 amino acids with the adjacent peptide. Pool 1 contained the peptides of the C-terminal half of N, pool 2 contained those of the N-terminal part. Splenocytes were tested for responses upon a brief in vitro stimulation with the peptides followed by staining for T cell markers and intracellular interferon (IFN)-g. As shown in FIG. 18 both vaccines induced robust CD8⁺ T cell responses to pool 2 while CD4⁺ T cells were only detectable in AdC6-N immunized mice; they were directed to peptides within pool 1.

Epitope Specificity of T Cells Induced by Priming with the AdC-N or AdC-gDN Vectors

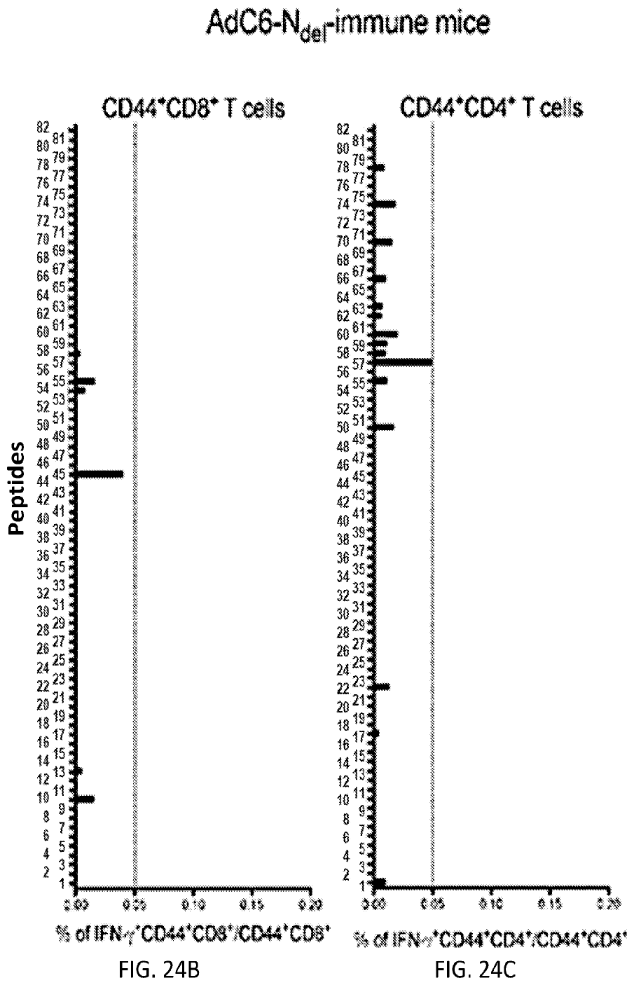

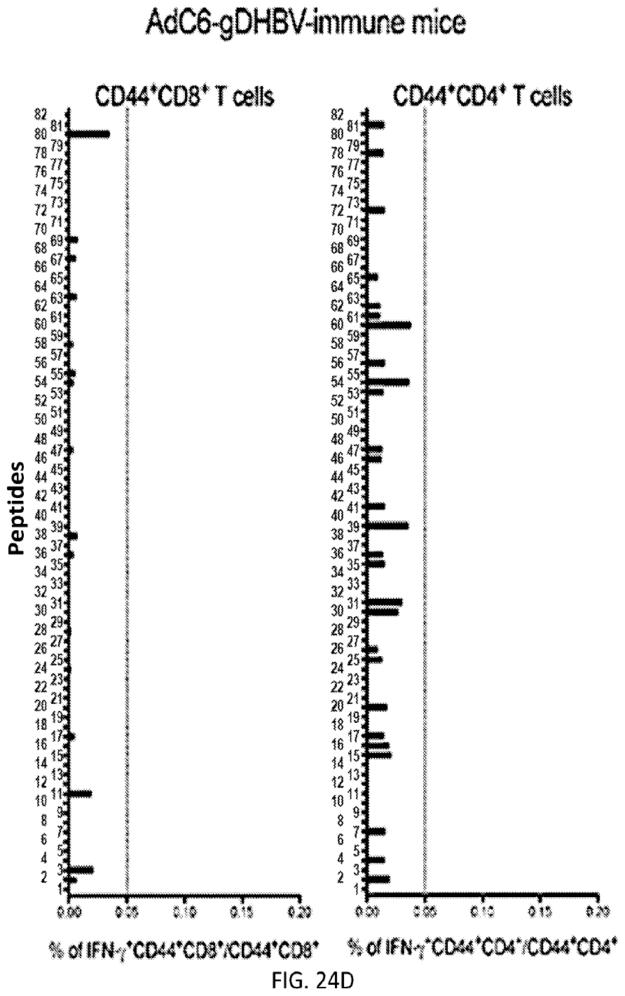

To determine the breadth of N-specific T cell responses, pooled splenocytes from vaccinated mice were stimulated with individual peptides spanning the entire N sequence and then stained for CD4, CD8 and CD44 surface markers and intracellular IFN-g. Cells from spleens of naïve mice served as controls and cells cultured with medium rather than a peptide were used to determine background activity. Frequencies of CD44⁺CD8⁺ or CD44⁺CD4⁺ T cells producing IGN-g in absence of peptides were subtracted from frequencies of the same cell subsets that produced IFN-g in response to an N peptide. Splenocytes from naïve mice had marginal responses against a few peptides and only the frequency of CD44⁺CD4⁺ T cells against peptides 64 slightly exceeded 0.05% (FIG. 24A). Therefore the limit of positive responses was set at frequencies of or above 0.2%. Accordingly, the Y-axis of each of the following graphs starts at 0.2. To further simplify the graphs results are only shown for peptides that scored positive for a given T cell subsets in at least one set of experiments. As additional controls to ensure that responses were not triggered by the vaccine carrier, tested mice were immunized with Ad vectors expressing parts of N from which either key epitopes had been deleted or which expressed only the N or C terminal part of the protein; lymphocytes from mice injected with the latter vectors were then tested with peptides to the deleted parts. Again, as shown in FIG. 24, these vectors failed to elicit a response to sequences that were not present in the vaccine insert (FIGS. 24B, 24C). To control for gD additional mice that had been injected 4 weeks earlier with an AdC6 vector expression gD fused with an irrelevant antigen were tested and again shown to be negative (FIG. 24D).

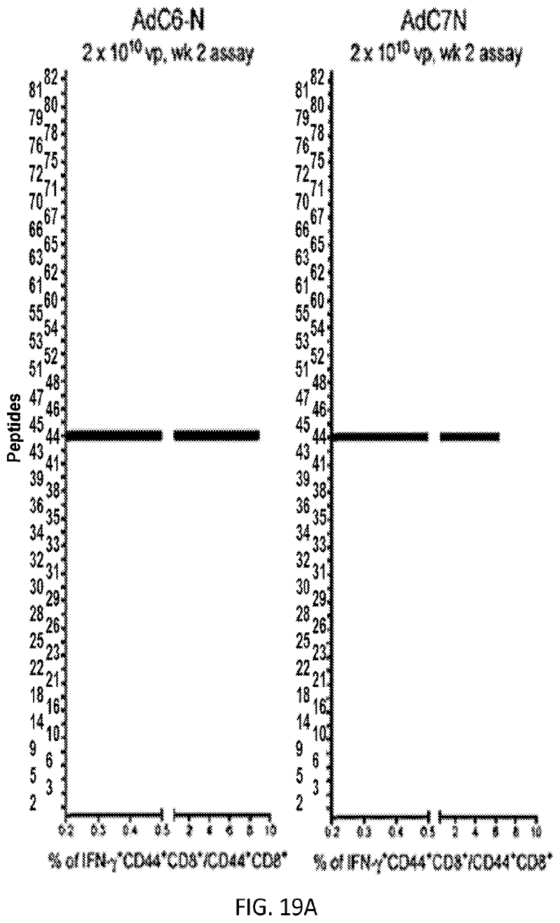

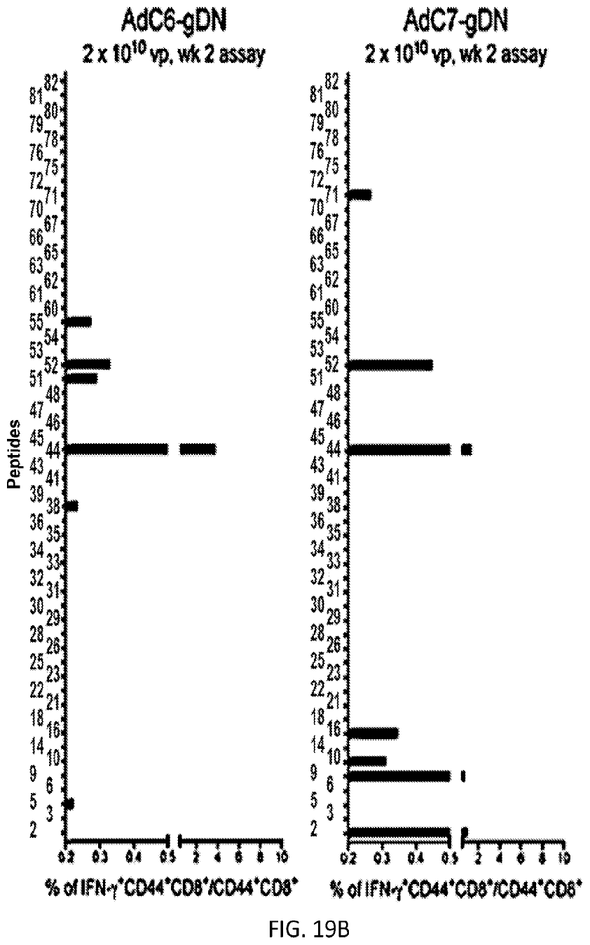

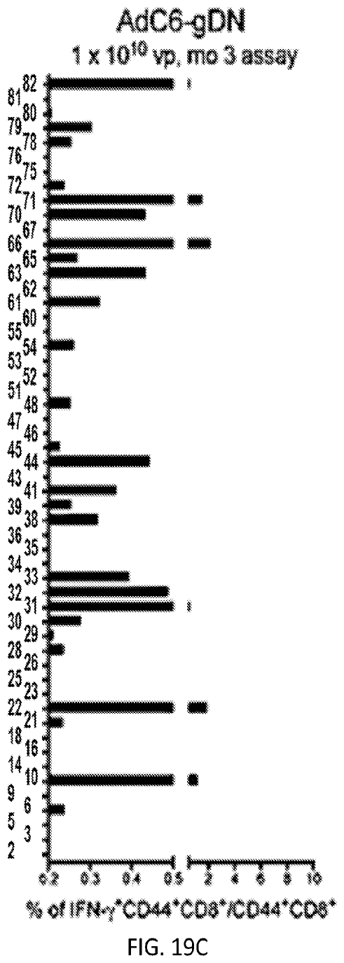

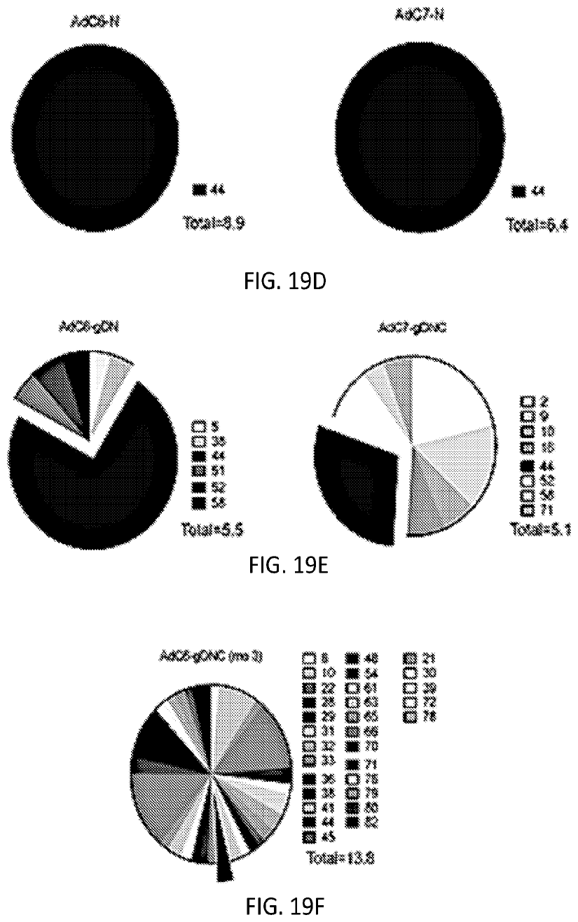

To determine CD8⁺ T cell epitopes that were recognized after priming, groups of 5 C57Bl/6 mice were immunized with 2×10¹⁰ vp of the AdC6-N, AdC7-N, AdC6-gDN or AdC7-gDN vectors and tested 2 weeks later. Another group was immunized with 1×10¹⁰ vp of the AdC6-gDN vector and tested 3 months later to determine response durability. As shown in FIGS. 19A, 19D the response to the AdC6-N and AdC7-N vector was monospecific and solely directed against peptide 44. Inclusion of gD into the vaccines broadened T cell responses to additional peptides although the response to peptide 44 remained dominant (FIGS. 19B, 19E). Surprisingly, reducing the AdC6-gDN vaccine dose to 1×10¹⁰ and delaying testing of splenocytes to 3 months after vaccination caused a further increase in the magnitude and breadth of the response as well as a shift in immunodominance away from peptide 44 towards other epitopes that were not recognized when T cells were tested at 2 weeks after vaccination (FIGS. 19C, 19F). Inclusion of gD did not increase the overall magnitude of CD8⁺ T cell responses when they were tested after 2 weeks as shown in FIGS. 19D-19F as the sum of responses to ‘unique’ epitopes. Sum of responses to all peptides excluded those to adjacent peptides that according to epitope predication express the same epitope. Frequencies were highest when tested 3 months after vaccination, which is unusual as T cell responses tend to contract once antigen-expressing cells have been eliminated. This unusual kinetics was only seen for vaccines expressing the gDN insert. When mice vaccinated with AdC6N at a moderate dose of 2×10¹⁰ vp were tested 10 weeks after immunization, the response remained monospecific for peptide 44 and the frequencies were slightly lower than when mice were tested after 2 weeks (5.6% vs. 8.9%, data not shown).

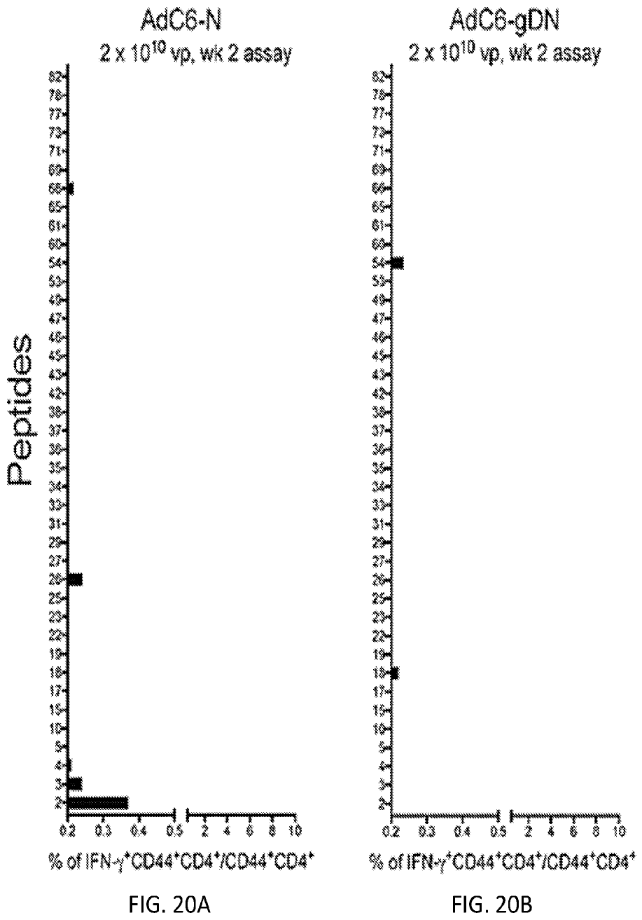

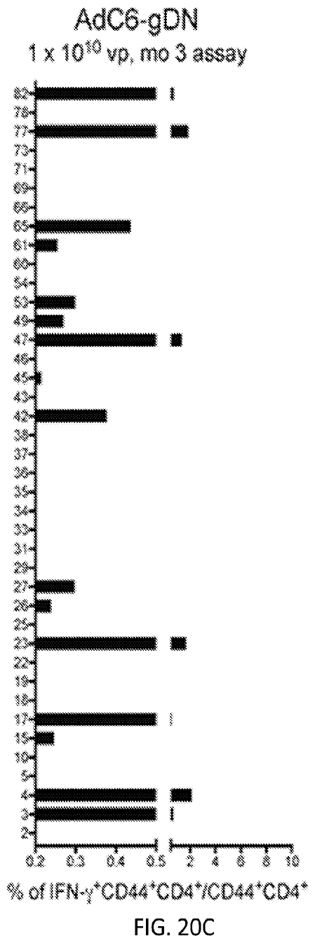

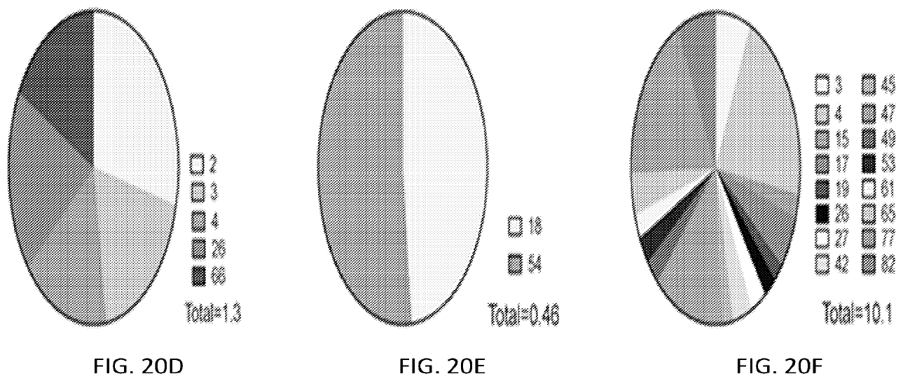

Splenocytes from mice injected with the AdC6 vectors were tested in parallel for CD44⁺CD4⁺ T cell responses to the peptides. CD4⁺ T cell responses to the AdC6-N (FIGS. 20A, 20D) and AdC6-gDN (FIGS. 20B, 20E) were low and directed to 5 vs. 2 peptides, respectively. As shown in FIGS. 19A-19F for CD8⁺ T cells, delaying testing till 3 months after vaccination with the AdC6-gDN vector not only augmented frequencies of CD4⁺ T cells but also markedly increased the breadth of responses (FIGS. 20C, 20F). Again, this was not seen after immunization with the AdC6-N vector (data not shown).

Epitope Specificity of CD8⁺ T Cells Induced by Prime-Boosting with the AdC-N Vectors

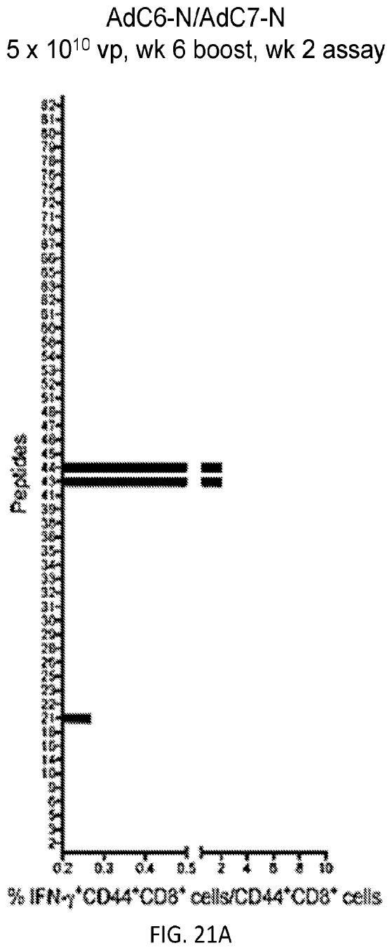

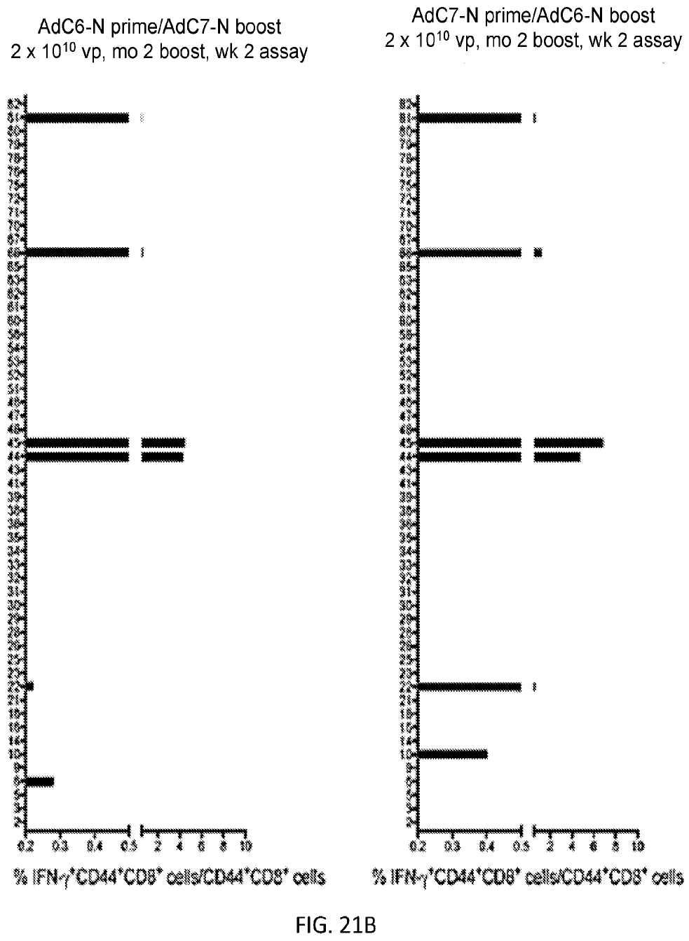

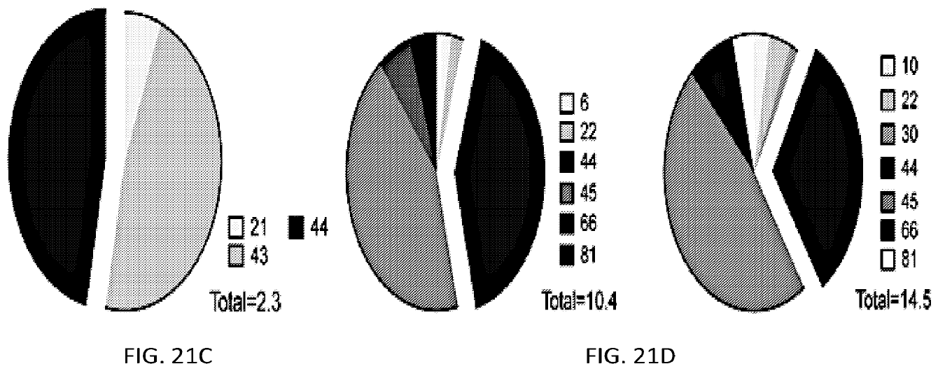

For this series of experiments groups of 5 C57Bl/6 mice were primed with AdC6-N at 5×10¹⁰ or 2×10¹⁰ vp. They were booster 6 weeks or 2 months later with the AdC7 vector expressing the same insert and used at the same doses. An additional group was immunized first with 2×10¹⁰ vp of the AdC7-N vector and then boosted 2 months later with the same dose of the AdC6-N vector. Splenocytes were tested 2 weeks after the boost for production of IFN-g to the individual N peptides as described above. As shown in FIG. 21A after the boost given within a 6-week interval using high doses of both the AdC6-N and AdC7-N vectors peptide 44 and the adjacent peptide 45 remained immunodominant and only one additional peptide, (i.e., peptide 21) scored a low response (FIGS. 21A, 21C). Reducing the dose of the two vaccines to 2×10¹⁰ vp and extending the interval between the two vaccine injections to 2 months increased magnitude and breadth of N-specific CD8⁺ T cell responses above those seen after priming (FIGS. 21B, 21D).

Epitope Specificity of CD8⁺ T Cells Induced by Prime-Boosting with the AdC-gDN Vectors.

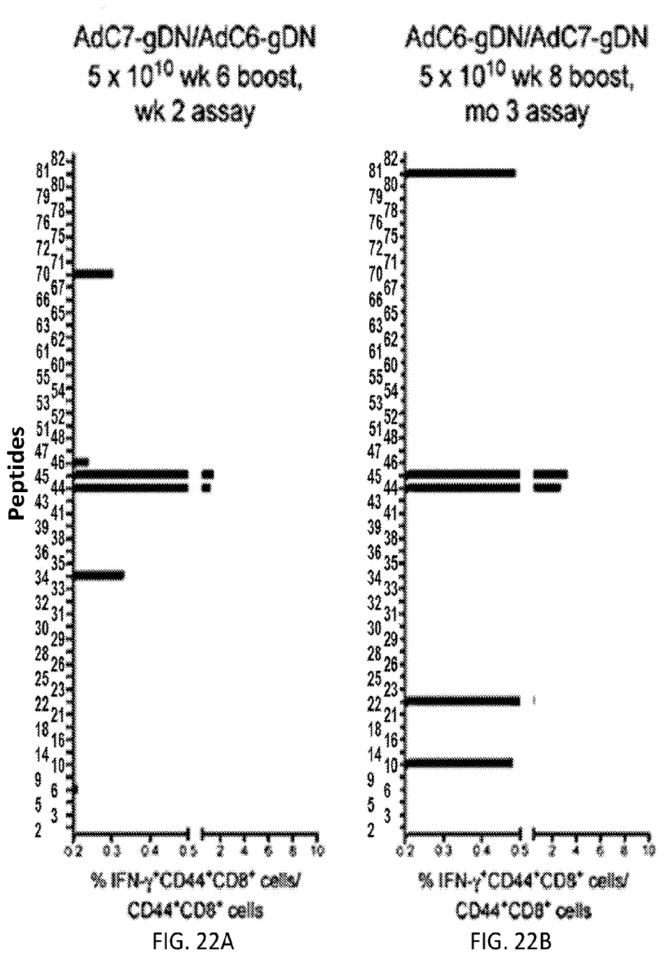

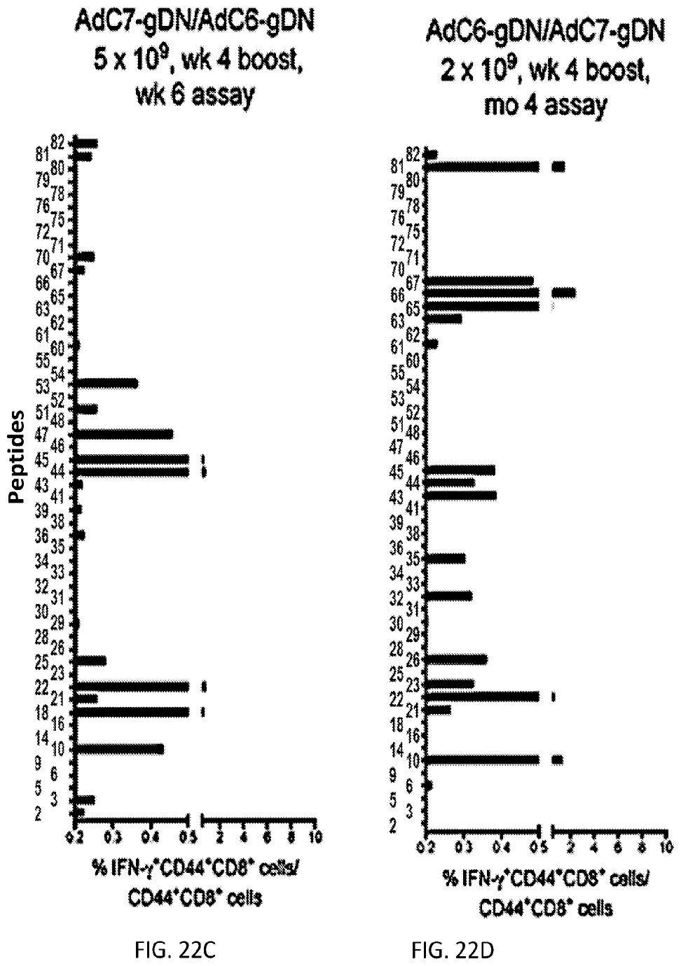

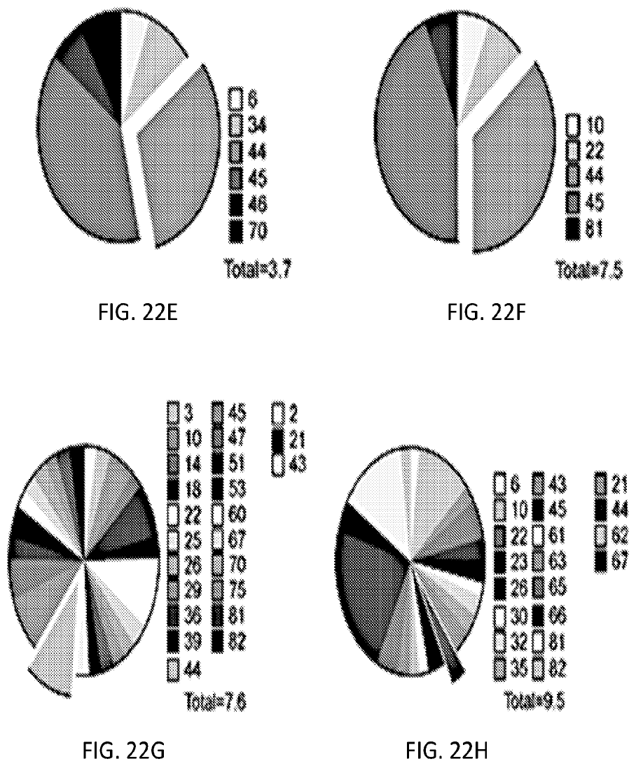

A similar experiment was conducted with the AdC-gDN vectors. One group was primed with a high dose of 5×10¹⁰ vp of the AdC7-gDN vector and boosted 6 weeks later with the same dose of the AdC6-gDN vector. Splenocytes were tested 2 weeks later (FIGS. 22A, 22E). The next group was primed with the same dose of the AdC6-gDN vector; boosting with the AdC7-gDN vector was delayed till week 8 and splenocytes were tested 3 months after the boost (FIGS. 22B, 22F). Both regimens induced responses that were dominated by those to peptides 44 and 45. The latter group also showed robust responses to three additional peptides and had overall higher frequencies of IFN-g-producing CD8⁺ T cells. One additional group was primed with 5×10⁹ vp of the AdC7-gDN vector and boosted 4 weeks later with the same dose of the AdC6-gDN vector; splenocytes were tested 6 weeks later (FIGS. 22C, 22G). A fourth group was primed with a low dose of 2×10⁹ vp of AdC6-gDN, boosted 4 weeks later with AdC7-gDN and tested 4 months later (FIGS. 22D, 22H). The breadth and magnitude of the CD8⁺ T response was markedly higher at the lower vector doses although they failed to exceed those of responses observed after a single immunization (FIGS. 19A-19F).

Epitope Specificity of CD4⁺ T Cells Induced by Prime-Boosting with the AdC-gDN Vectors.

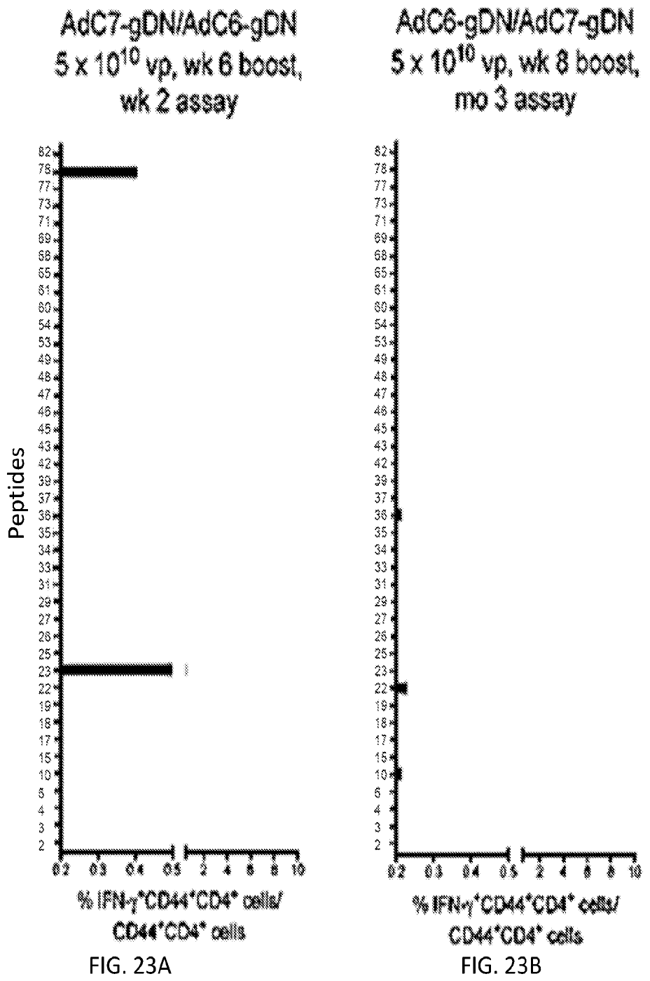

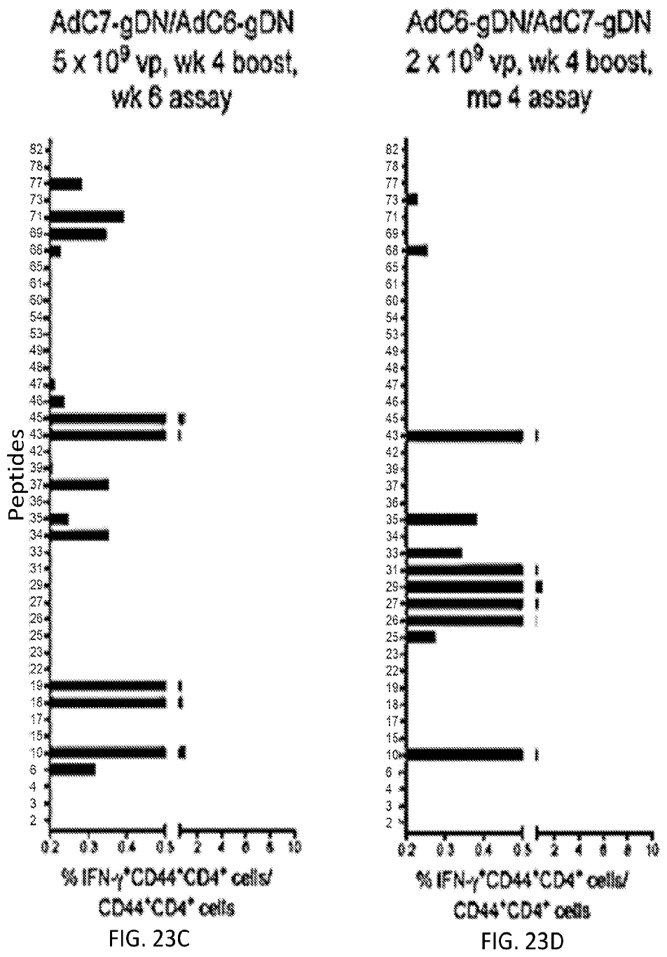

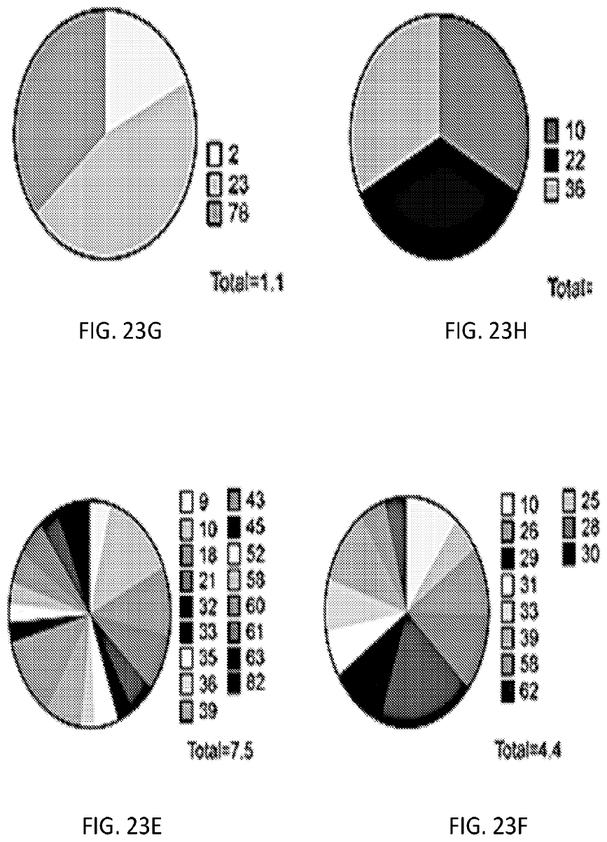

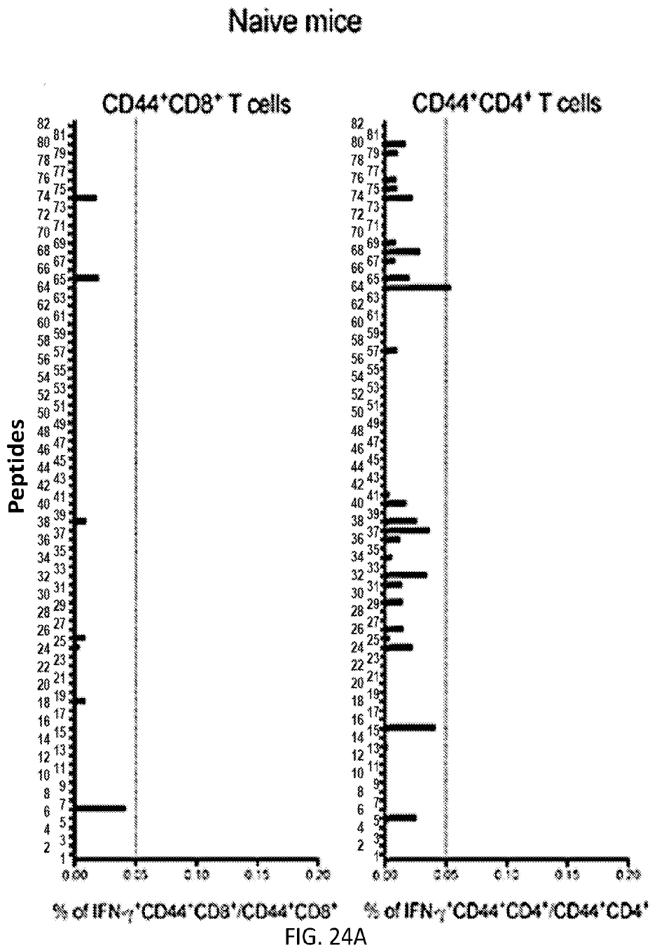

N-specific CD4⁺ T cells were only analyzed upon prime-boosting with the gDN expressing AdC vectors using again different vector doses and different intervals between prime-boosting and testing. The 5×10¹⁰ vp dose of AdC vectors with a 6-week interval between prime and boost resulted in low and narrow responses by 2 weeks after the boost (FIGS. 23A, 23E). Reversing the order of the vectors, increasing the interval between the two injections to 8 weeks and the testing to 3 months after the final injection diminished the response further (FIGS. 23B, 23F). Reducing the vector dose to 5×10⁹ vp per injection and boosting after 4 weeks induced strong and broad CD4⁺ T cell responses when tested 6 weeks after the last injection (FIGS. 23C, 23G). Similar results were obtained with a vaccine regimen where the order of the vectors used at the same doses was reversed and splenocytes were tested 4 months after the 2^{nd} injection (FIGS. 23D, 23H).

### MHC Class I Epitope Binding and MHC Class I and II Epitopes Selected by Prediction Software

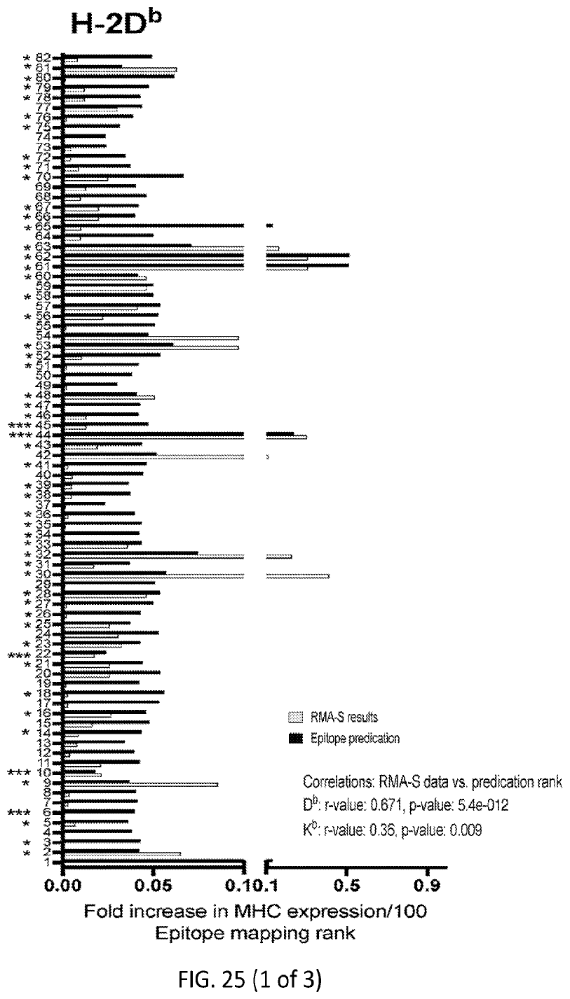

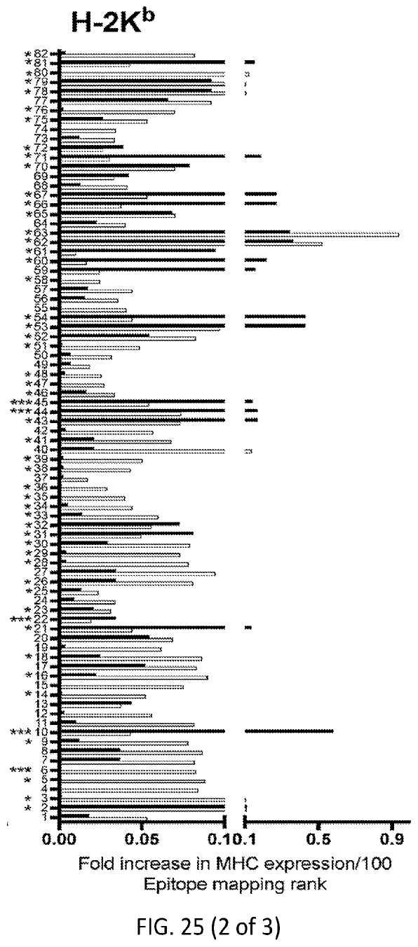

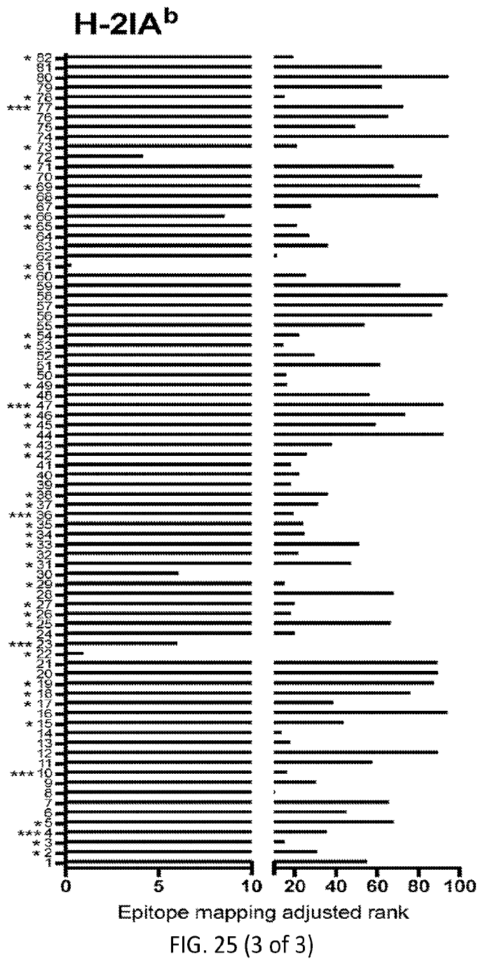

To validate results for CD8⁺ T cells, each peptide was tested for binding to RMA-S cells. These cells lack the transporter associated with antigen processing (TAP) and therefore fail to express MHC class I antigens unless a D^{b} or K^{b} binding peptide is added to stabilize surface expression of MHC class I molecules, which can then be detected by staining and flow cytometry. In addition, epitope predication software was used to determine which peptides were likely to bind to MHC class I or II antigens of H-2^{b}. Prediction of MHC class I binding peptides correlated well with results obtained with the RMA-S cells (see FIG. 25) and with most peptides that scored positive in the CD8⁺ T cell assays. Magnitude of T cell responses was not necessarily linked to high prediction scores or the most pronounced increased in MHC class I expression on RMA-S cells. For example, peptide 6 was highly positive in some of the T cell assays but scored poorly with the prediction software or in the RMA-S cell assay while peptide 63, which scored very high in these two analyses, elicited only modest CD8⁺ T cell responses. MHC class II epitope prediction was less foretelling for positive results in the CD4⁺ T cell assays; two of the highest-ranking peptides, i.e., peptides 30 and 72 failed to trigger responses while others that ranked poorly (>10 for the adjusted rank) triggered high production of IFN-g by CD4⁺ T cells (e.g., peptides 47 and 77). The same software was used to assess if these results obtained in a pre-clinical models have likely relevance for recognition of SARS-CoV-2 by human T cells. As shown in Table 4 most of the peptides recognized by H-2^{b} mice also scored as HLA binders:

| | | | | Binding to HLA* | | | | |
|---|---|---|---|---|---|---|---|---|
| | | | SEQ | | | SEQ | | |
| | Amino | | ID | | | ID | | Percentile |
| Peptide | Acids | Peptide Sequence | NO: | Allele** | Core*** | NO: | Score | Rank |
| 2 | 6-20 | PQNQRNAPRITFGGP | 8 | HLA-B*15:01 | NQRNAPITF | 96 | 0.67 | 0.16 |
| 9 | 41-55 | RPQGLPNNTASWFTA | 15 | HLA-B*57:01 | QGLPNNTSW | 97 | 0.77 | 0.24 |
| 10 | 46-60 | PNNTASWFTALTOHG | 16 | HLA-A*68:02 | NTASWFTAL | 98 | 0.87 | 0.03 |
| 21 | 101-115 | MKDLSPRWYFYYLGT | 27 | HLA-B*07:02 | SPRWYFYYL | 99 | 0.85 | 0.07 |
| 30 | 146-160 | IGTRNPANNAAIVLQ | 36 | HLA-A*30:01 | GTRNPANNA | 100 | 0.57 | 0.09 |
| 31 | 151-165 | PANNAAIVLQLPQGT | 37 | HLA-A*68:02 | NNAAIVLQL | 101 | 0.24 | 0.55 |
| 32 | 156-170 | AIVLQLPQGTTLPKG | 38 | HLA-B*15:01 | LQLPQGTTL | 102 | 0.79 | 0.07 |
| 44 | 216-230 | DAALALLLLDRLNQL | 50 | HLA-A*02:01 | LLLDRLNQL | 95 | 0.96 | 0.02 |
| 45 | 221-236 | LLLLDRLNQLESKMS | 51 | HLA-A*02:01 | LLLDRLNQL | 95 | 0.96 | 0.02 |
| 61 | 301-315 | WPQIAQFAPSASAFF | 67 | HLA-B*15:01 | AQFPSASAF | 103 | 0.99 | 0.01 |
| 63 | 311-326 | ASAFFGMSRIGMEVT | 69 | HLA-A*11:01 | ASAFFGMSR | 104 | 0.80 | 0.08 |
| 66 | 326-340 | PSGTWLTYTGAIKLD | 72 | | | | | |
| 67 | 331-345 | LTYTGAIKLDDKDPN | 73 | | | | | |
| 70 | 346-360 | FKDQVILLNKHIDAY | 76 | HLA-B*15:01 | LLNKHIDAY | 105 | 0.87 | 0.03 |
| 71 | 351-365 | ILLNKHIDAYKTFPP | 77 | HLA-B*15:01 | LLNKHIDAY | 105 | 0.87 | 0.03 |
| 81 | 401-415 | DDFSKQLQQSMSSAD | 87 | | | | | |
| | Amino | | | | | | | Adjusted |
| Peptide | Acids | Peptide sequence | | Allele | Core | | Score | Rank |
| 3 | 11-25 | NAPRITFGGPSDSTG | 9 | | | | | |
| 22 | 106-120 | PRWYFYYLGTGPEAG | 28 | HLA-DRB1*04: | PRWYFYYLGTGP | 106 | 0.77 | 0.83 |
| | | | | 05 | EA | | | |
| 23 | 111-125 | YYLGTGPEAGLPYGA | 29 | | | | | |
| 61 | 301-315 | WPQIAQFAPSASAFF | 67 | HLA-DRB1*09: | PQIAQFAPSASAFF | 107 | 0.01 | 0.01 |
| | | | | 01 | | | | |
| 66 | 326-340 | PSGTWLTYTGAIKLD | 72 | HLA-DRB1*07: | PSGTWLTYTGAIK | 108 | 0.40 | 0.43 |
| | | | | 01 | L | | | |
| 82 | 406-420 | QLOQSMSSADSTQA | 88 | HLA-DRB1*07: | PSGTWLTYTGAIK | 108 | 0.40 | 0.43 |
| | | | | 01 | L | | | |

*Potential binding to common HLAs was determined using an epitope prediction tool
**HLA allele that scored the highest binding
***Peptide recognized by the corresponding HLA

### Conservation of T Cell Epitopes Between SARS-CoV-2 Isolates

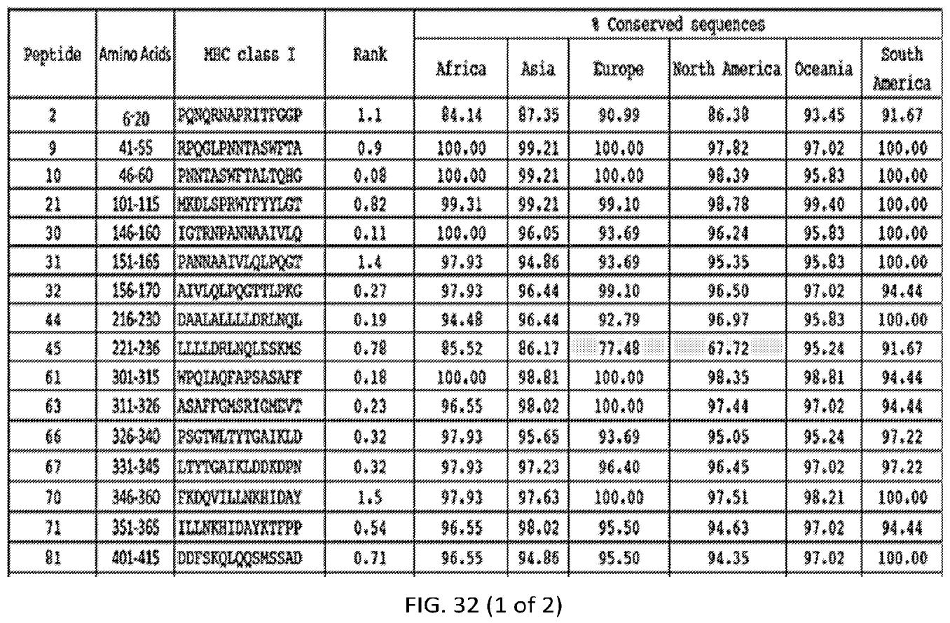

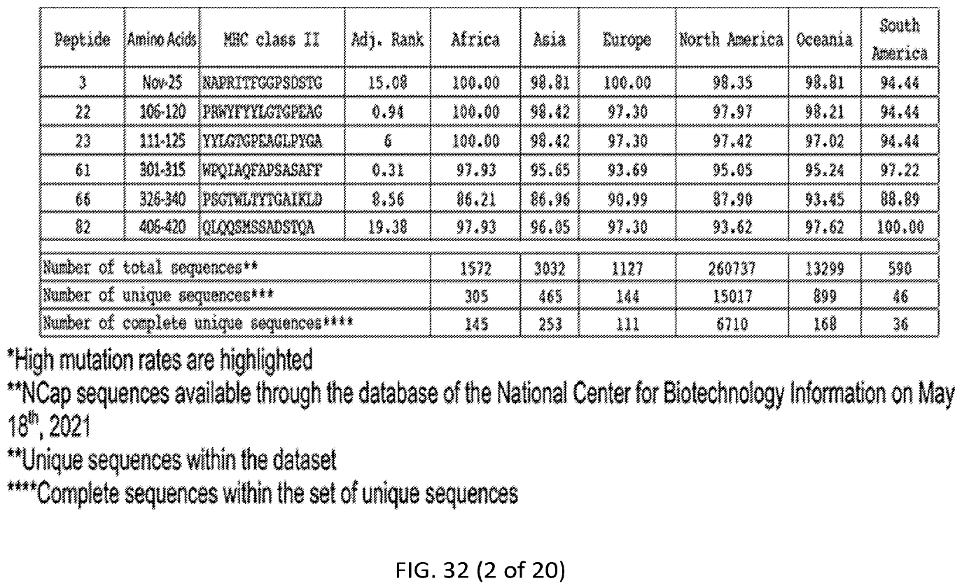

Viral escape can dampen vaccine efficacy. The degree of changes within the epitopes recognized by vaccine-induced T cells was determined by analysing 7423 unique and complete isolates from across the globe for presence of the epitopic N peptide sequences. As shown in FIG. 32 the amino acid sequences for peptides that identified the different epitopes were highly conserved:

North America, from where most sequences originated, showed the highest degree of variability for both CD4⁺ and CD8⁺ T cell epitopes while isolates from South America and Oceania were the least variable. One peptide, peptide 45, which represents amino acids 221-235 of N, showed exceptionally high variability in >30% of North American and >20% of European SARS-CoV-2 isolates. The variability of the 221-235 sequence within the available sequence set was further analysed. In most regions but for North America and Europe, variability of individual amino acids within sequence 221-235 mirrored the overall variability of N. The most common mutations in all regions but for Oceania and South America were a serine (S) to phenylalanine (F) exchange in position 235 or a methionine to isoleucine exchange in position 234 (Table 5B):

| | | Frequencies of mutation within peptide 45 (SEQ ID NO: 51)* | | | | | |
|---|---|---|---|---|---|---|---|
| Peptide 45 | SEQ ID | | | | N. | | South |
| Sequence 221-235* | NO: | Africa | Asia | Europe | America | Oceania | America |
| LLLLDRLNQLESKMS | 45 | 85.52 | 86.17 | 77.48 | 67.72 | 95.24 | 91.67 |
| LLLLDRLNQLESKMF | 91 | 9.66 | 6.72 | 15.32 | 15.47 | 0.00 | 2.78 |
| LLLLDRLNQLESKIS | 92 | 2.76 | 4.74 | 6.31 | 14.29 | 0.60 | 5.56 |
| LLLLDRLNQFESKMS | 93 | 0.00 | 0.00 | 0.90 | 0.21 | 1.79 | 0.00 |
| LLLLDRLNHLESKMS | 94 | 0.00 | 0.79 | 0.00 | 0.36 | 0.60 | 0.00 |

*Table shows the wildtype amino acid sequence 221-235 of SARS-CoV-2 N in row 3 followed by the most frequent variants and their frequencies in different regions.
**Mutated amino acids are underlined

The unique sequences for the position of changes within the N 221-235 sequence were screened. Over 90% of the mutations involved amino acids 234 and 235 (Table 5C):

| | | Types and frequencies of mutations | | |
|---|---|---|---|---|
| Amino | | | Number of | % of |
| acid | | | isolates*** | isolates**** |
| position | Wt* | Mutations** | 5165 | 69.62 |
| 221 | L | F, V | 14 | 0.19 |
| 222 | L | M | 2 | 0.03 |
| 223 | L | | | |
| 224 | L | F, P | 5 | 0.07 |
| 225 | D | Y, H, E | 3 | 0.04 |
| 226 | R | K | 8 | 0.11 |
| 227 | L | F, V | 11 | 0.15 |
| 228 | N | H, S, R, L | 9 | 0.12 |
| 229 | Q | F, H, L, K | 18 | 0.24 |
| 230 | L | F | 25 | 0.34 |
| 231 | E | T, D | 2 | 0.03 |
| 232 | S | G, I, N, R, C | 62 | 0.84 |
| 233 | K | I, R | 12 | 0.16 |
| 234 | M | I, V, L, T | 1019 | 13.74 |
| 235 | S | F, L, P, A, C, Y | 1130 | 15.23 |

*wt-wild-type, the column shows the amino acid sequence of SARS-CoV-2 N the original isolate.
**The column shows the amino acid changes at the different positions in all sequences from Table 1A.
***The column shows the number of sequences with mutations at the different positions, numbers of unmodified sequences are shown on top.
****The columns provide the same information as the column to the left as percentages over unique complete sequences.

An analyses with epitope prediction software showed that sequence 221-235 is expected to bind to HLA-A*02:01, HLA-A*02:03, HLA-A*02:08 and HLA-B*08:01 with a rank of 0.8-0.9; nevertheless, the core epitope (LLLDRLNQL)(SEQ ID NO: 95) for these HLA types does not involve the commonly mutated amino acids 234 and 235, which are also unlikely to play a role in binding to human MHC class II molecules (tools.iedb.org/main/).

### DISCUSSION

Vaccines can stop a pandemic by achieving herd immunity. Those based on attenuated viruses tend to be highly effective commonly providing protective immunity for life. They induce a full spectrum of adaptive immune responses unlike inactivated viruses or protein vaccines that in general stimulate antibodies and CD4⁺ T cells but are poor inducers of CD8⁺ T cells. Most inactivated viral vaccine require periodic booster immunizations because protective immune responses wane, as exemplified by rabies vaccines, or the virus escapes vaccine-induced protective immunity by accumulating mutations, such as influenza viruses. Genetic vaccines including RNA vaccines or E1-deleted Ad vectors are perceived by the adaptive immune system like attenuated viruses; they induce de novo synthesis of the vaccine antigen in transduced cells, which promotes its processing for association of immunogenic peptides to both MHC class I and II antigens. Genetic vaccines unlike attenuated viral vaccines only express a single viral antigen, which reduces the response breadth.

COVID-19 vaccines that have been licensed for emergency use thus far all express the viral spike proteins. In pre-clinical experiments they showed induction of T and B cells and protection against disease. Early clinical trials demonstrated safety, which was confirmed after mass vaccination campaigns had reached millions of humans causing very rarely serious adverse events such as anaphylactic reactions after the RNA vaccines or the thrombocytopenia with thrombosis syndrome also known as vaccine-induced prothrombotic immune thrombocytopenia, a potentially fatal auto-immune response that has been reported to affect ˜1 in 250,000 recipients of some of the Ad vector vaccines. The early trials reported induction of T and B cell responses. Most importantly phase III trials confirmed that the vaccines were highly efficacious in preventing serious disease or death. Ad vector vaccines have advantages over RNA vaccine; they are less costly and more heat stable. One potential disadvantage is that neutralizing antibodies to Ad vaccine carriers that are either already present in humans due to natural infections or that are induced by vaccination may blunt vaccine immunogenicity. Regarding efficacy of the different Ad vector vaccines, the Johnson and Johnson vaccine, a single dose vaccine that uses HAdV-26 as the vaccine carrier showed 66.3% efficacy against infection overall with 74.4% efficacy in the US. Efficacy in countries such as South Africa and Brazil, where viral variants have become dominant remained high at 64% and 68%, respectively. Sputnik V, which is a two-dose vaccine that uses an HAdV-26 vector prime followed 4 weeks later by a boost with an HAdV-5 vector reported 91.6% efficacy, while the AstraZeneca vaccine, which is based on an AdC vector used in a homologous prime-boost regimen prevents symptomatic COVID-19 disease in 76% of the vaccine recipients.

Correlates of protection against COVID-19 remain ill-defined. A protective role for neutralizing antibodies is supported by nonhuman primate studies, which showed protection upon adoptive transfer of antibodies. These early pre-clinical results were confirmed in humans and included trials, which showed that infusion of SARS-CoV-2-specific monoclonal antibodies benefits patients with mild to moderate COVID-19 disease. Antibody-mediated resistance to SARS-CoV-2 infection is likely to be dampened over time by mutations of the receptor-binding domain of the viral spike protein as already suggested in pre-clinical studies by loss of protection of mice that received plasma containing antibodies against an early US isolate prior to challenge with the B7.1.1.7 variant that evolved in the UK or the B.1.351 variant from South Africa.

The importance of CD8⁺ T cells was revealed by a study that showed breakthrough infections in SARS-CoV-2-immune rhesus macaques upon CD8⁺ T cell depletion stressing that these cells contribute to protection upon a natural infection and presumably upon vaccination. This might be supported further by the finding that humans, who are in general protected against a reinfection upon mild or asymptomatic COVID-19 commonly have robust T cells responses in absence of detectable antibodies.

A set of AdC vaccines that are suited for heterologous prime-boost vaccination was developed. The two vaccine carriers, AdC6 and AdC7 both belong to family E of adenoviridea and represent distinct serotypes so that antibodies induced by one of the vectors fail to neutralize the other, which is also one of the potential advantages of the Sputnik V over the AstraZeneca vaccine. In addition, many humans especially in less develop countries have robust titers of pre-existing neutralizing antibodies to human serotypes of Ad viruses such as HAdV-5 and HAdV-26 (33), which by blocking cell transduction and production of the SARS-CoV-2 antigen by the vaccine may blunt responses. In contrast, neutralizing antibodies to AdC viruses are rare in humans and those who carry them in general have low titers. As vaccine antigen, N was selected under the assumption that this protein will over time show less variability than spike, which with increasing numbers of SARS-CoV-2-immune humans will more and more face neutralizing antibody-mediated selection pressure. Furthermore, N has already been shown experimentally to induce robust T cell responses in naturally infected humans and responses cross-react with other human coronaviruses that have been circulating for several decades.

T cells are primarily directed against a small set of so-called immunodominant epitopes within a viral protein and viruses can evade such responses by mutational escape as was shown in chronic infections with for example hepatitis C virus or human immunodeficiency virus. To broaden CD8⁺ T cell responses one set of AdC vectors expresses the N protein within HSV-1 gD, which by blocking the early BTLA-HVEM checkpoint promotes activation of T cells to subdominant epitopes. As shown by the data presented herein, N presented in its wild-type form by either the AdC6 or AdC7 vectors induced after a single immunization very focused CD8⁺ T cell responses to only one epitope carried by peptide 44 while the gDN fusion protein vaccines elicited responses to several other epitopes. Surprisingly, CD8⁺ and CD4⁺ T cell responses to the gDN—but not the N-expressing vectors increased over time so that by 3 months after a single vaccine dose both breadth and magnitude of responses were markedly higher than after 2 weeks. CD4⁺ T cell responses when tested at 2 weeks after immunization were low but like CD8-T cell responses increased and gained breadth over time. As a rule, T cell responses peak within 1-2 weeks after exposure to antigen and then, once the antigen has been removed, most effector cells die and responses contract. Ad vectors like Ad viruses tend to persist at low levels and maintain effector T cell responses for prolonged periods of time while T cell transitioning into memory is delayed (48-50). Nevertheless, this does not fully explain the unusual response kinetics to the AdC-gDN vaccines. Results from previous experiments that use AdC vectors expressing a different antigen within gD suggest that thus checkpoint inhibitor may in part play a role in delaying peak responses.

Booster immunization with the AdC-N vectors increased the breadth of the CD8⁺ T cell responses to several additional epitopes but was relatively ineffective for AdC-gDN vectors Prime-boosting with high doses of the AdC-gDN vectors decreased responses while lower doses were performed better potentially indicating that excessive doses of the vaccines caused prolonged activation of T cells rendering them susceptible to activation-induced cell death upon re-exposure to antigen. These results are reminiscent of those obtained in clinical trials with the AstraZeneca vaccine, which showed higher efficacy at a lower vaccine dose and if the booster immunization was given at 3 months rather than within 6 weeks after priming.

The vaccine regimens differed in timing of the boost or the analyses. The results do not suggest that the former plays a major role while the latter clearly allows for heightened and broadened CD8⁺ but not necessarily CD4⁺ T cell responses. Lack of a more robust booster response may reflect the previously described delay in T cell memory formation due to vector persistence suggesting that a single dose regimen may suffice for further clinical development of Ad vector-based T cell vaccines. The N sequences that were recognized by the vaccine-induced CD8⁺ and CD4⁺ T cells were highly conserved within SARS-CoV-2 isolates from around the globe and most showed less than 5% variability. One clear exception was amino acid sequence 221-235 present in peptide 45 (LLLLDRLNQLESKMS)(SEQ ID NO: 51) which showed 32.5% and 22.3% variability within North American and European isolates, respectively. Most of the mutations involved amino acids 234 and 235, which do not appear to play a role in binding to common HLAs suggesting that viral fitness rather than pressure from cell-mediated immunity may have been the cause for selection of these mutants. One study reported selection pressure on amino acid 232 of SARS-CoV-2 N; this amino acid shows 0.84% variability in the sequence panel but again according to epitope predication is unlikely to be part of a dominant CD8⁺ T cell epitope. The same paper reported that the amino acid in position 13 of N, which in the original isolate is a proline showed the highest degree of variability. Using the sequence panel, also it was observed that this amino acid vas frequently exchanged mainly by a leucine. Mutations were more common in the Americas and Asia (>4.5% of all sequences) but comparatively rare in Oceania (1.2% of all sequences). The proline is part of an epitope with good binding to HLA-B*07:02, which is lost upon its replacement with leucine suggesting that this mutation may have been caused by T cell-mediated selection pressure. HL A-B*07:02 is rare in the Americas but frequent in the UK suggesting that the initial selection may have taken place prior to spread of the variant into the Americas.

In summary, AdC vector expressing the N protein within HSV-1 gD induce robust and sustained T cell responses. They also induce very broad responses to multiple epitopes within the protein, which is essential to prevent viral escape through mutations. Combined with vectors expressing the viral spike protein for induction of neutralizing antibodies they may prolong vaccine-induced protection and guard against mutational escape, which is especially critical for less developed countries, where access to vaccines is limited.

### Example 6: Testing of Two Serologically Distinct Chimpanzee-Origin Adenovirus Vectors Expressing Spike of SARS-CoV-2

Here, pre-clinical results with two serologically distinct AdC vectors expressing the S protein of an early SARS-CoV-2 isolate are described. The results show that the vaccines induce S protein-specific antibodies including VNAs. Antibody responses are sustained for at least 6 months and increase after a boost: the magnitude of the booster response depends on vaccine dose and interval between the two immunizations and is enhanced by using heterologous vectors. VNAs generated by a single vaccine dose show limited cross-reactivity with the S protein of viral variants, which markedly increases after a boost. Vaccinating mice sequentially with two AdC vectors expressing either the original S protein, or a version in which the receptor binding domain (RBD) had been mutated to resemble the South African variant only marginally improved cross-reactivity of VNAs to additional viral variants.

### Material and Methods

Cell lines. HEK 293 cells and VeroE6 cells were grown in Dulbecco's Modified Eagles medium (DMEM) supplemented with 10% fetal bovine serum (FBS) and antibiotics in a 5% C02 incubators. BHK-21/WI-2 cells were grown in DMEM supplemented with 5% FBS and antibiotics at 5% C02.
Expression of ACE-2. Cells diluted to 1×10⁶ per microtiter plate well were incubated for 45 min at room temperature with a 1 in 100 dilution of a mouse IgG2a antibody to human/hamster ACE-2 (clone 171606 R&D Systems, Minneapolis, MN). After washing, cells were incubated for 45 min with an FITC-labeled goat-anti mouse IgG and a live cell stain. After washing, cells were analyzed by flow cytometry.
Generation of and quality control AdC-S vectors. The cDNA sequence for S_{SWE} of SARS-CoV-2 was obtained from the laboratory of Dr. Elledge, Massachusetts General Hospital, Boston, MA (GenBank: QIC53204.1). Sequencing revealed a frame-shift mutation that was corrected by site-directed mutagenesis. The corrected S gene was cloned into a shuttle vector and from there into the viral molecular clones of AdC6 and AdC7. Viral vectors were rescued, expanded, and purified on HEK 293 cells. Viral DNA was isolated and tested by restriction enzyme digest for presence and integrity of the inserts. Viral titers were determined by spectrophotometry. Yields for AdC6-S and AdC7-S were 1.4×10¹³ and 2.5×10¹³ respectively upon expansion in 2×10⁸ HEK293 cell. Genetic stability was determined by restriction enzyme digest of purified viral DNA after 12 sequential passages of the vectors.

To allow for rapid exchange of the RBD sequence an AgeI site was inserted into position 1640 of the S gene which changes the nucleotide but not the amino acid sequence of S. The variant RDB sequences including flanking regions and convenient restriction enzyme sites were synthesized (Genscript Biotech, Piscataway, NJ) and cloned into the S_{SWE} gene using restriction enzymes BsrGI which cuts at base pair 1104 and Age1 of the S_{B1.351} sequence and BseGI and NheI, which cuts at base pair 2015 of the S_{B1.1.7} and S_{B.1.617.2} sequences.

### Generation of VSV-S Vectors

VSV pseudotyped with S of SARS-CoV-2 were generated in BHK-21/WI-2 cells using a the ΔG-GFP (G*ΔG-GFP) rVSV kit (Kerafast, Boston, MA, USA) and S sequences cloned into an expression plasmid under the control of the CMV promoter. BHK21/WI-2 cells were plated into a 6 well plate, the next day cells were first transfected with 1 μg/well of the paT7 plasmids using polyethyleneimine (PEI) (Polysciences, Inc. Warrington, PA); 2 hours cells were infected with a predetermined optimal amount of ΔG-GFP (G*ΔG-GFP) rVSV. After a 24-hour incubation at 37° C. in a 5% C02 incubator, supernatants were harvested, aliquoted, and stored at −80° C.

### Protein Expression

The AdC-S vectors were tested for protein expression upon transfection of HEK 293 cells with 1000 vp of the vectors for 48 hours at 37° C. Cells were lysed in RIPA buffer supplemented with a 1% μl protease inhibitor (Santa Cruz Biotechnology Inc., Dallas, TX). A 15 ul of lysate was resolved on 12% SDS-PAGE and transferred to a polyvinylidene difluoride (PVDF) membrane (Merck Millipore, Burlington, MA). The membrane was blocked in 5% powder milk overnight in 4° C. The primary antibody to S diluted to 1:1000 in saline (clone ABM19C9, Abenomics, San Diego, CA) was added for 1 h at room temperature. Membranes were washed with 1×TBS-T prior to incubating with HRP-conjugated goat anti-rabbit secondary IgG (ab6721, Abcam, Cambridge UK) for 1 h at room temperature. In parallel the membrane was probed with a mouse monoclonal IgG antibody to ß-actin (Sc-47778, Santa Cruz Biotechnology, Dallas, TX) as a loading control for 1 h at room temperature. The loading control antibody was probed with HRP-conjugated goat anti-mouse secondary IgG (SAB3701047, Sigma, St. Louis, MO) for 1 hr at room temperature. Membranes were washed 3 times with 1×TBS-T. The developing agent Super Signal West Pico Chemiluminescent (Thermo Fisher Scientific, Waltham, MA) was added. Membranes were shaken in the dark for 5 min, dried and developed.

### Mice and Mouse Procedures

Female C57Bl/6 mice (6-8 weeks of age) were purchased from the Jackson Laboratories (Bar Harbor, ME). Outbred 6-8-week-old female ICR mice were obtained from Taconic Biosciences (New York, NY). Mice were housed at the Wistar Institute Animal Facility. All mouse procedures followed approved protocols. Mice were vaccinated intramuscularly (i.m.) with the AdC vectors diluted in 200 μl of sterile saline. Mice were bled from the saphenous vein and blood was collected into 4% sodium carbonate and Liebowitz's-15 (L-15) medium. Serum was isolated 30 min later upon a 10 min centrifugation of tubes at 14000 rpm.

### ELISA

Sera from individual mice were tested for S-specific antibodies by ELISA on plates coated overnight with 100 μl of a mixture of S1 and S2 (Native Antigen Company, Kidlington, UK) each diluted to 1 μg/ml in bicarbonate buffer. The next day plates were washed and blocked for 24 hours at 4° C. with 150 μl of a 3% BSA-PBS solution. Sera were diluted in 3% BSA-PBS and 80 μl of the dilutions were added to the wells after the blocking solution had been discarded. Plates were incubated at room temperature for 1 hour and then washed 4× with 150 μl of PBS. An alkaline-phosphate-conjugated goat anti-mouse IgG antibody (Sigma-Aldrich, St Louis, MO) diluted to 1:1500 in 3% BAS-PBS was added at 60 μl/well for 1 hour at room temperature. Plates were washed 4 times and substrate composed of phosphatase substrate tables (Sigma-Aldrich, St Louis, MO) diluted in 5 ml of diethanolamine buffer per tablet was added. Plates were read in an ELISA reader at 405 nm. Coated wells only receiving the substrate served to determine background. Background data were subtracted from the experimental data. Titers were calculated based on linear regression with a Y-intercept of 0.

VNA Assay with VSV-S

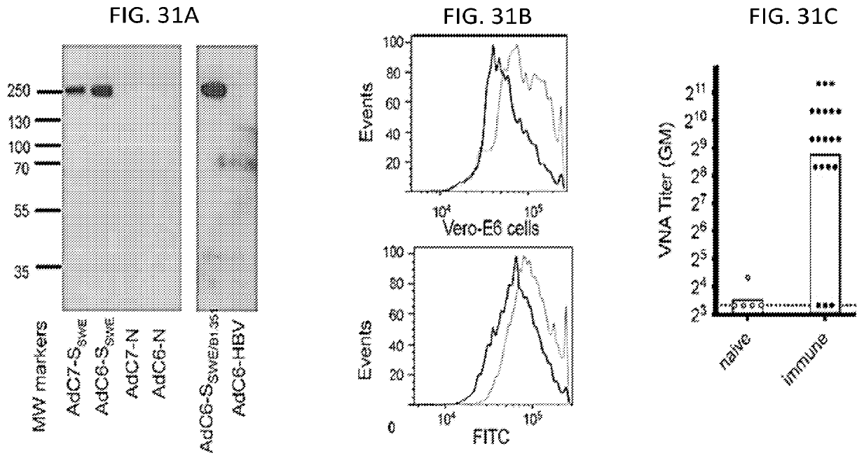

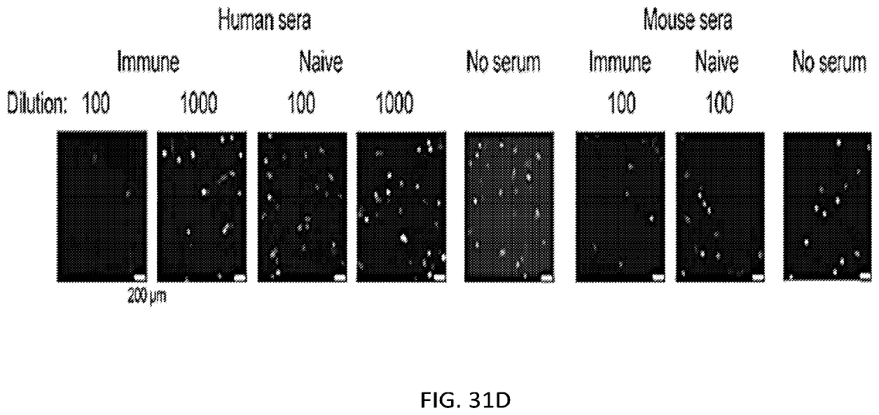

VSV-S vectors was initially titrated on BHK-21/WI-2 cells. The cells were diluted to 4×10⁵ cells/ml in DMEM with 5% FBS and 5 μl were of the cell dilution was added to each wells of Terasaki plates. The next day serial dilutions of VSV-S vectors were added to duplicate wells. The following day numbers of fluorescent cells/well were counted with a fluorescent microscope (FIG. 31D). For the neutralization assay a dose of VSV that infected ˜50-100 cells/well was selected.

For the neutralization assay, BHK-21/WI-2 cells were plated into Terasaki plate wells as described above. The following day, sera were serially diluted in DMEM with 5% FBS and then incubated with VSV-S diluted in the same medium for 90 min at room temperature under gentle agitation starting with a serum dilution of 1 in 40 or 1 in 100. 5 μl aliquots of the mixtures were transferred onto BHK-21/WI-2 within the Terasaki plate wells. Each serum dilution was tested in duplicates, an additional 4-6 wells were treated with VSV-S that had been incubated with medium rather than serum. Plates were incubated for 24-48 hours and then numbers of fluorescent cells were counted. Titers were set as the last serum dilution reduced numbers of green-fluorescent cells by at least 50%.

The COV-PosSet-S from RayBiotech (Peachtree Comers, GA), which contains 20 human samples from individuals that recovered from COVID-19 or from uninfected individuals was used to validate the neutralization assay.

### ACE Binding Inhibition Assay

Sera were tested for inhibition of ACE binding to the RBD of S by the Anti-SARS-CoV-2 Neutralizing Antibody Titer Serologic Assay Kit from Acro Biosystems (Newark, DE) following the manufacturer's instructions. A positive standard with a known concentration provided by the kit was used to extrapolated anti-S antibody concentrations in μg in the mouse sera.

### Results

### Generation and Testing of AdC-S Vaccine Vectors

E1-deleted replication-defective AdC6 (SAdV23) and AdC7 (SAdV24) vectors expressing the S protein of an early SARS-CoV-2 isolate from Sweden called SARS-CoV-2/human/SWE/01/2020 (AdC-S_{SWE}, GenBank number: QIC53204) were developed. In addition, an AdC6 vector expressing the same S protein in which the RBD sequence was modified to incorporate the E484K and N501Y mutations of the B1.351 South African variant (AdC6-S_{SWE/B.351}) was generated. Vectors were produced with the help of available molecular Ad clones and upon expansion, purification, titration and passing quality control assays they were tested for protein expression upon transfection of HEK-293 cells. As shown in FIG. 31A both types of AdC vectors express proteins of the expected size that bind the anti-S protein antibody.

Generation and Testing of Vesicular Stomatitis Virus (VSV) Vectors Pseudotyped with the S Protein of SARS-CoV-2.

To allow for testing of VNAs, several green fluorescent protein (GFP)-expressing VSV vectors that were pseudotyped with S protein of SARS-CoV2 (VSV-S) were developed. One carries the same S protein as the vaccine vectors (VSV-S_{SWE}). In a second the S_{SWE} sequence was modified to incorporate the E484K and N501Y RBD mutations of the B1.351 South African variant (VSV-S_{SWE/B.351}). Others express either the S protein with N501Y and P681H RBD mutations present in the B1.1.7 UK variant (VSV-S_{SWE/B1.17}), or L452R and D614G mutations of the Indian B.1.617.2 delta variant (VSV-S_{SWE/B.1.617.2}).

To establish a neutralization assay with the VSV-S vectors, 3 different cell lines including the commonly used VeroE6 cell line was tested but it was decided on baby hamster kidney (BHK)-21/WI-2 cells, as they gave more consistent results. BHK-21/WI-2 cells express the ACE2 receptors that is used by SARS-CoV2 for cell entry as was shown by immunofluorescent staining and flow cytometry (FIG. 31B). VSV-S_{SWE} virus transfection of BHK-21/WI-2 cells is neutralized by human antibodies from individuals, who experienced a SARS-CoV-2 infection but not by antibodies from sera of noninfected human controls (FIG. 31C). It is also neutralized by sera from S protein-immune but not naïve mice (FIG. 31D).

### Antibody Responses to Different Doses of AdC-S Vectors

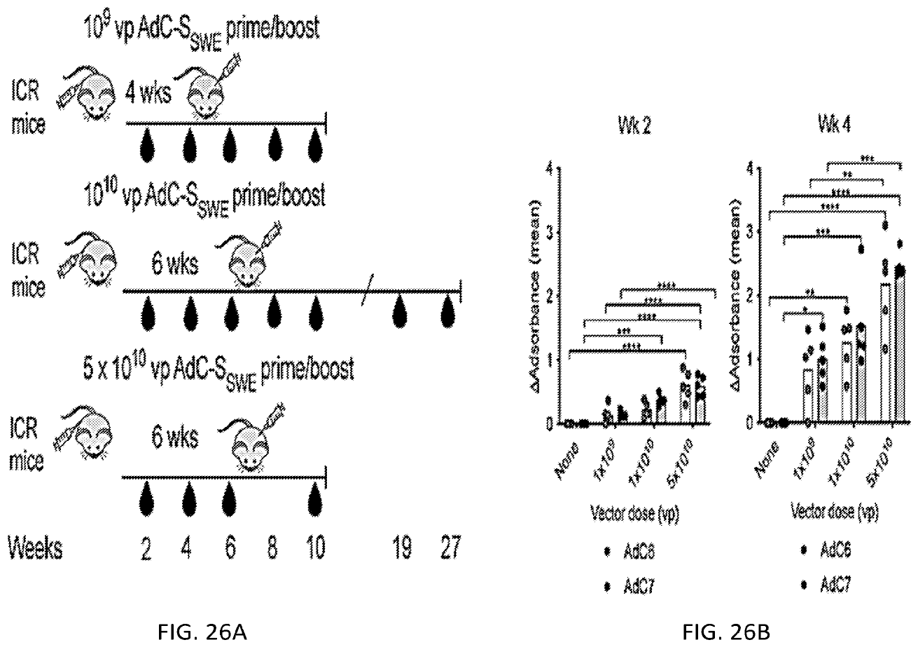

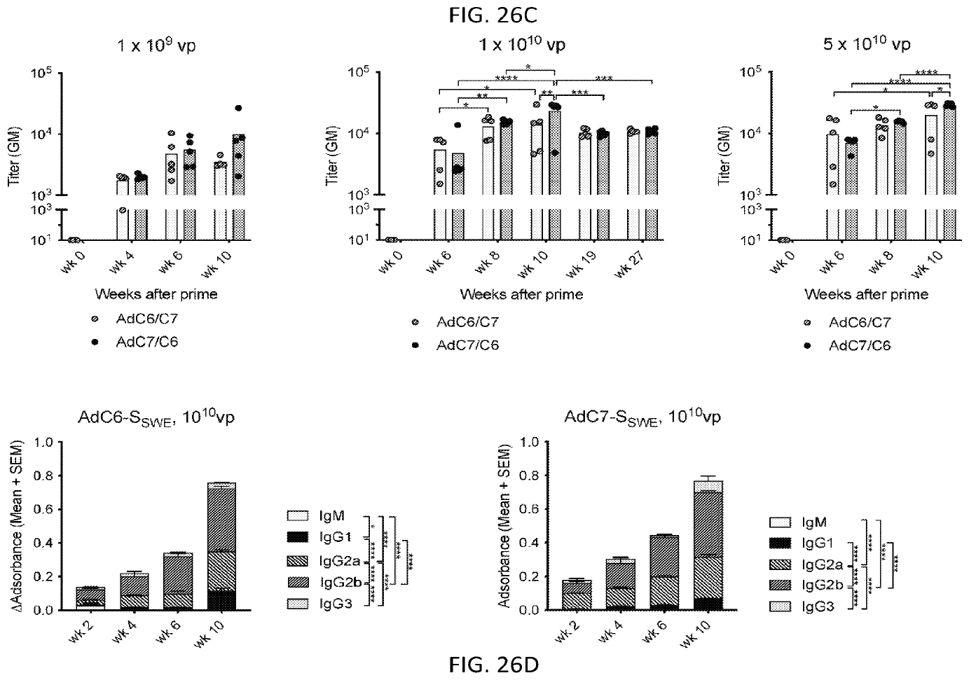

Groups of outbread ICR mice (5/group) were immunized with 10⁹, 10¹⁰ or 5×10¹⁰ virus particles (vp) of of the AdC6-S_{SWE} and AdC7-S_{SWE} vectors. Mice were bled 2 and 4 weeks later. Mice that received the lowest vector dose were boosted 4 weeks after the prime; the other mice were boosted 6 weeks after the prime. All mice were boosted with heterologous AdC vectors given at the same doses that had been used for priming. Mice were bled 2 weeks after the boost. Mice that received the 1×10¹⁰ vp doses were kept for 27 weeks following the prime and bled periodically to determine duration of antibody responses (FIG. 26A). Sera from individual mice were tested in duplicates at various dilutions by an enzyme-linked immunosorbance assay (ELISA) on a mixture of commercially available S1 and S2 proteins of SARS-CoV-2. As displayed in FIG. 26B, which shows absorbance values obtained by ELISA for individual sera tested at a 1:200 dilution, by 2 weeks after the prime mice in the high dose group developed antibodies while by 4 weeks all animals but for one mouse in the low dose AdC6-S_{SWE} group mounted detectable S protein-specific antibody responses. Response magnitude was dose dependent. As shown in FIG. 26C, which displays titers calculated by linear regression, responses significantly increased by 2 or 4 weeks after the boost in most groups but for the low dose AdC6-S_{SWE} group that had received the 2^{nd} injection more rapidly by 4 weeks after the prime. Antibodies as tested for the 10¹⁰ vp vaccine groups persisted for at least 27 weeks after the prime with a small decrease in the group that had been primed with AdC7-S_{SWE} followed by a boost with AdC6-S_{SWE}. Antibody isotypes were tested using pooled sera from different time points at a 1:100 dilution. Responses were dominated by antibodies of IgG2 isotypes indicative of the T helper cell type 1 response (FIG. 26D).

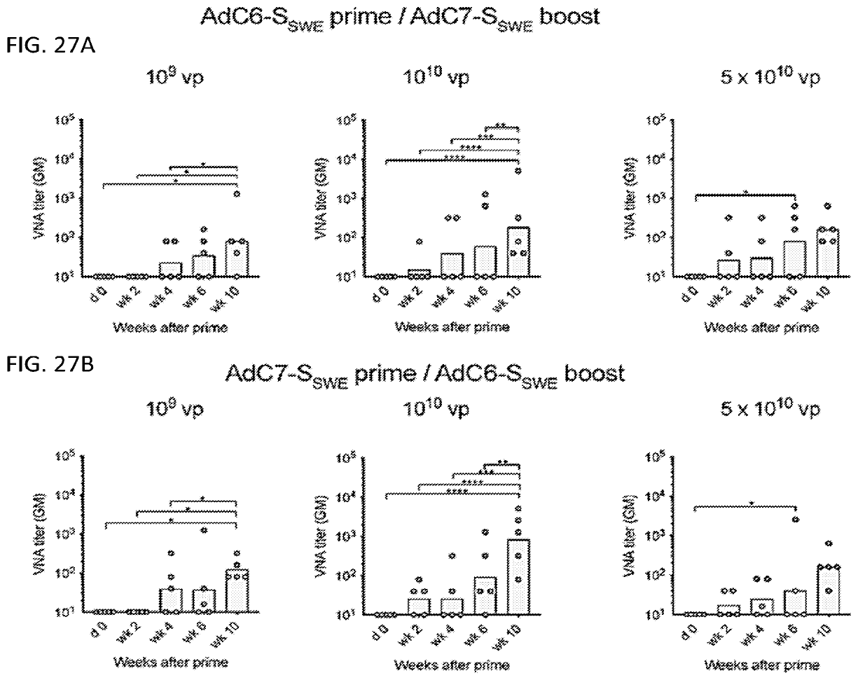

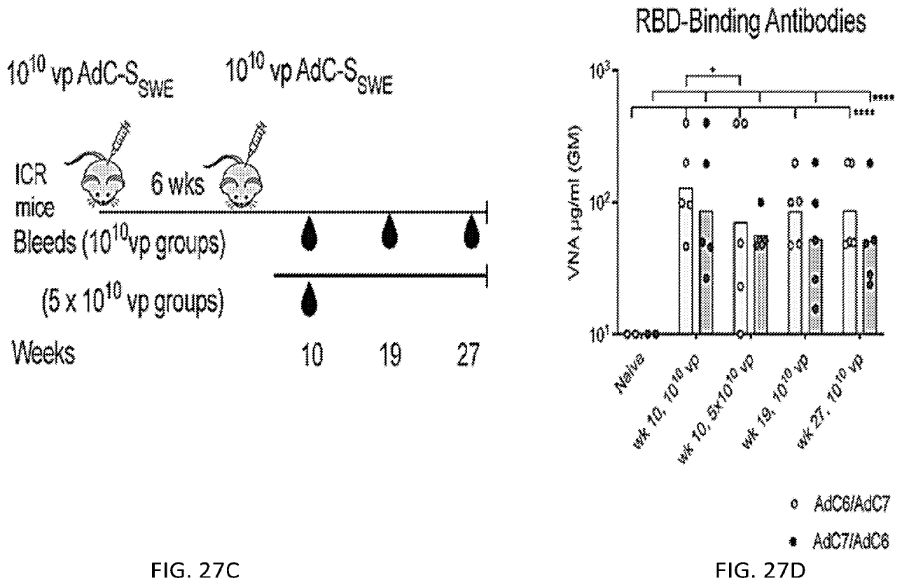

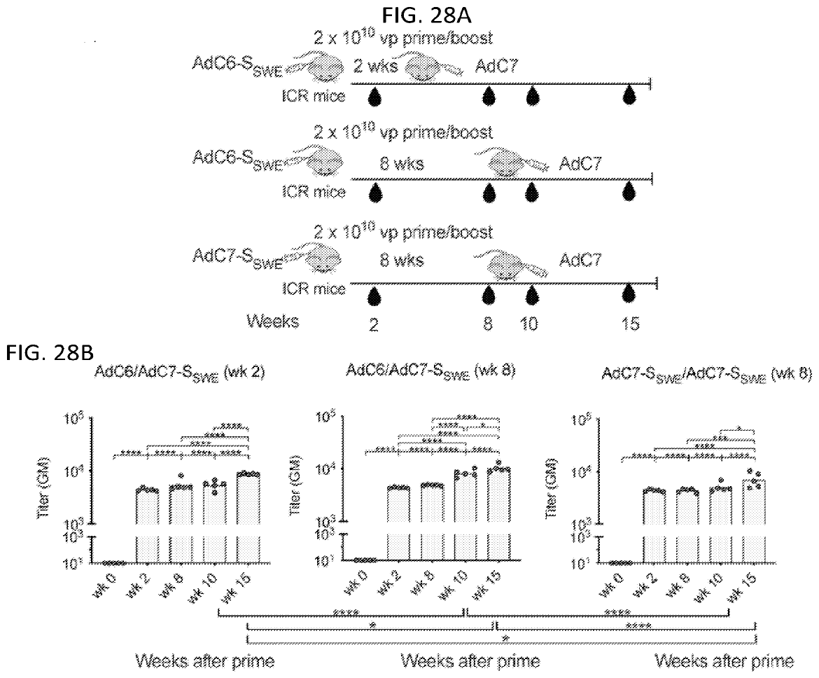

The same set of sera were tested by a neutralization assay with the VSV-S_{SWE} vector. After the boost by week 10 all mice but for one in the low dose AdC6-S_{SWE} group developed VNAs with geometric mean (GM) titers ranging from 1:80 in the low dose AdC6-S_{SWE} prime group to 1:840 in the intermediate dose AdC7-S_{SWE} prime group (FIGS. 27A, 27B). To confirm induction and longevity of SARS-CoV-2 VNAs a surrogate neutralization assay was used that tests sera for inhibition of binding of human ACE2 to S protein's RBD using samples from mice that had been primed with 10¹⁰ or 5×10¹⁰ vp AdC-S_{SWE} and then boosted with the same doses of the heterologous AdC vectors (FIG. 27C). As shown in FIG. 27D, all mice developed RBD-specific antibodies by week 10, which were maintained without significant declines through week 27. In the group that received the AdC6-S_{SWE} prime followed by the AdC7-S_{SWE} boost the 10¹⁰ vp dose resulted in significantly higher antibody titers compared to the 5×10¹⁰ vp dose.

### Responses to Different AdC-S Vaccine Regimens

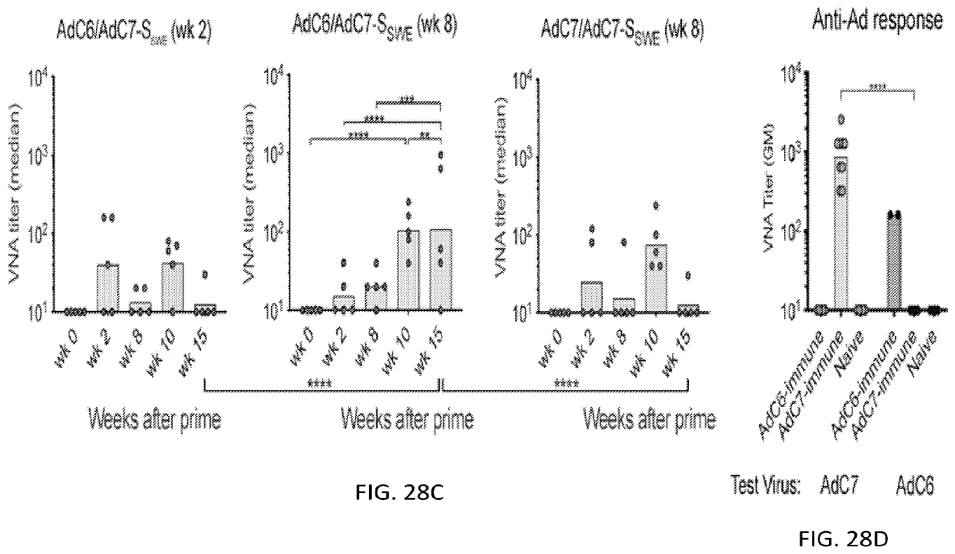

Different intervals between the prime and boost were used and the sequential use of homologous vs. heterologous AdC-S_{SWE} vectors were explored. For these experiments 3 groups of 5 ICR mice were tested (FIG. 28A). The 1^{st} group was primed with 2×10¹⁰ vp of AdC6-S_{SWE}. Mice were boosted 2 weeks later with the same dose of AdC7-S_{SWE}. The 2^{nd} group received the same vaccines at the same dose and in the same order but the boost was delayed till week 8. The 3^{rd} group was primed with 2×10¹⁰ vp of AdC7-S_{SWE} and then boosted 8 weeks later with the same AdC7-S_{SWE} vector used at the same dose. Mice were bled periodically and antibody titers to S protein were determined by ELISA. As shown in FIG. 28B the early boost was initially ineffective but mice showed a modest increase in titers by week 15. The same was seen for mice that received the homologous AdC7-S_{SWE}/AdC7-S_{SWE} boost. Highest increases in responses were obtained with the heterologous boost given 8 weeks after priming.

More pronounced differences were obtained with the neutralization assay (FIG. 28C), which showed that the early boost increased antibody titers 8-fold by week 10 (8 weeks after the boost), which then rapidly contracted. The homologous boost only achieved a 4-fold transient increase in VNA titers while the heterologous boost given 8 weeks after priming resulted by week 10 in an ˜8-fold increase and then by week 15 in a 26-fold increase. The less potent recall response of the homologous prime boost was most likely caused by Ad-specific VNAs that developed after the prime against the homologous but not the heterologous AdC vector (FIG. 28D).

### Responses in Young and Aged Mice

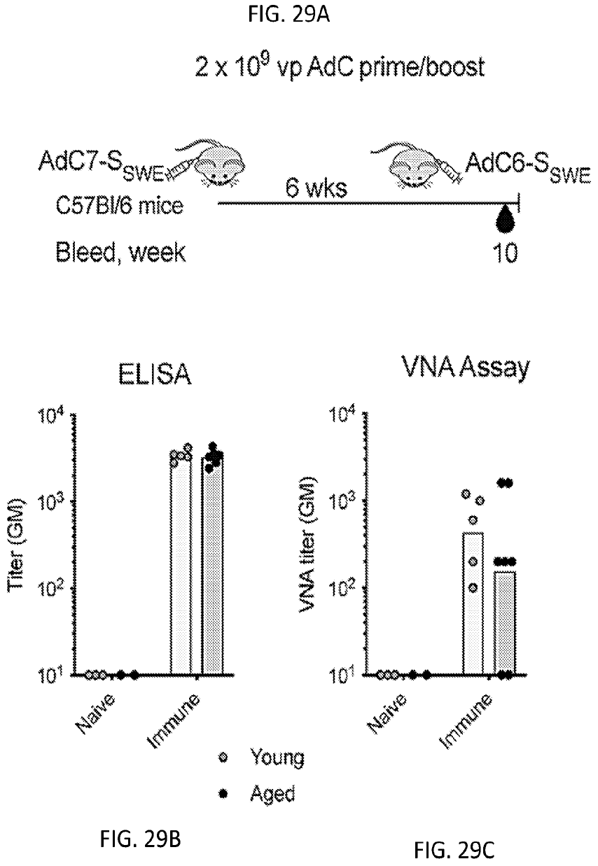

An experiment in young (6-8 weeks of age, n=5) and aged (1-2 years of age, n=7) C57Bl/6 mice was conducted. Mice were immunized with a low dose of 2×10⁹ vp of the AdC7-S_{SWE} vector and boosted 6 weeks later with 2×10⁹ vp of the AdC6-S_{SWE} vector (FIG. 29A). Naive young (n=3) and aged (n=2) mice were used as controls. Sera of individual mice were tested 2 weeks after the boost by an ELISA (FIG. 29B) and a VSV-S_{SWE} virus neutralization assay (FIG. 29C). Young and aged mice mounted comparable antibody responses when tested by ELISA. VNA titers tended to be higher in young mice although this failed to reach significance. All of the young mice developed VNAs while 2 out of 7 aged mice failed to respond.

### Cross-Reactivity of Vaccine-Induced Antibodies to SARS-CoV-2 Variants

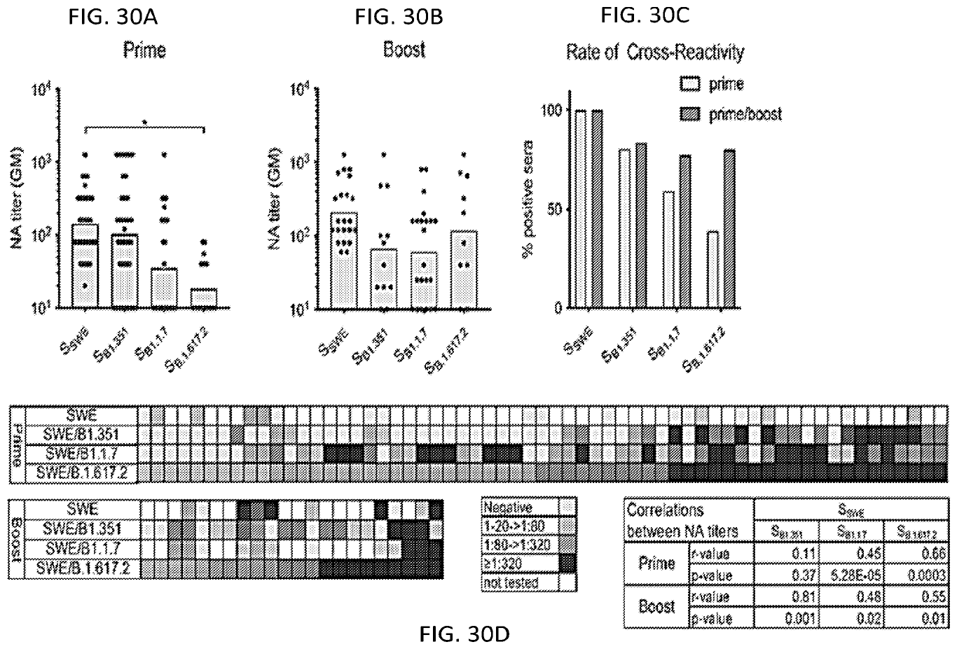

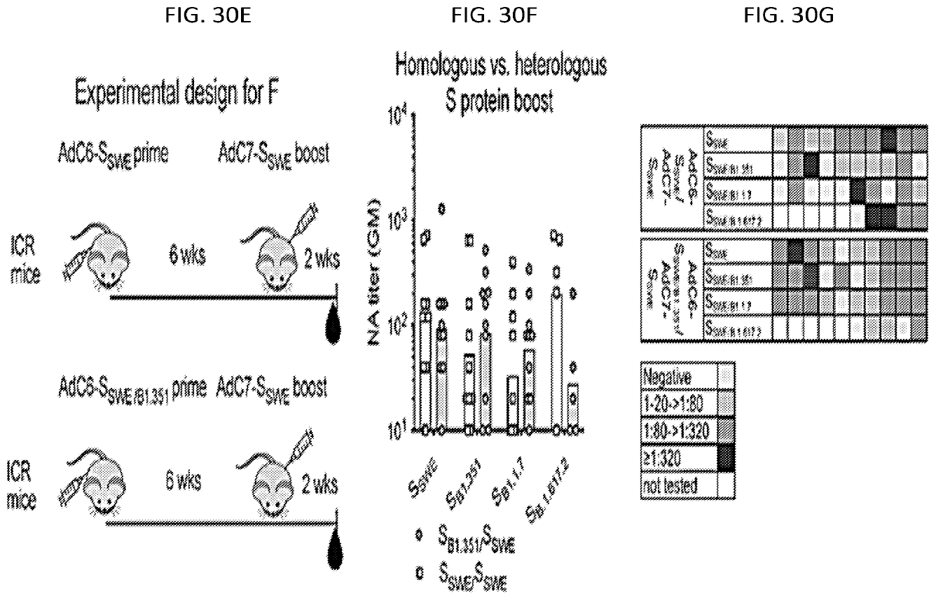

To test if the AdC-S vaccines induced antibodies that cross-neutralize viral variants, sera from ICR mice that had been immunized once or twice with AdC-S_{SWE} vectors and had shown positive neutralization of the VSV-S_{SWE} vectors was selected, and their cross-reactivity with VSV vectors pseudotyped with different versions of S protein carrying RBD mutations found in viral varians of concern was tested. A total of 61 or 22 sera harvested after priming (FIG. 30A) or boosting (FIG. 30B), respectively, were tested for cross-reactivity against VSV-S_{SWE/B1.351} and VSV-S_{SWE/B1.1.7} while 18 or 10 sera, respectively were tested against VSV-S_{SWE/B.1.617.2}. After priming, VNA titers were lower against VSV-S_{SWE/B.1.1.7} and VSV-S_{SWE/B.1.6.17.2} than against VSV-S_{SWE} and the latter difference reached significance. After the boost VNA titers became comparable. Sera with higher titers were, as expected, more cross-reactive than those with low titers. Accordingly, after priming antibody titers to S_{SWE} showed significant positive correlations to those against S_{SWE/B.1.1.7} and S_{SWE/B.1.6.17.2} and after the boost significance extended to correlations between S_{SWE} and S_{SWE/B1.351}. A direct comparison of titers may not be valid as S-pseudotyped VSV vectors may exhibit differences in their sensitivity to antibody-mediated neutralization that could relate to factors other than the S protein sequences. Therefore, the rates of responsiveness were also compared. All sera neutralize VSV-S_{SWE}. Response rates against the variants were markedly lower after a single immunization: 20%, 40% and 60% of sera failed to neutralize VSV-S_{SWE/B.351}, VSV-S_{SWE/B.1.1.7}, or VSV-S_{SWE/B.1.617.2}, respectively. Reactivity markedly increased after the boost when ˜80% of sera showed broad cross-reactivity (FIGS. 30C, 30D).

To assess if cross-reactivity could be increased by using a prime boost regimen with vectors expressing two different S proteins, 10 mice were primed either with 10¹⁰ vp of AdC6-S_{SWE} or AdC6-S_{SWE/B.351} vectors. Mice were boosted 6 weeks later with 10¹⁰ vp of AdC7-S_{SWE} and sera were tested 2 weeks later for neutralization of the different VSV-S vectors (illustrated in FIG. 30E). Although there was a trend for higher VNA titers towards the VSV-S_{SWE/B.1.17} and VSV-S_{SWE/B.1.6.17.2} vectors upon sequential immunization with Ad vectors expressing two different S proteins, differences were subtle and failed to reach significance (FIGS. 30F, 30G).

### DISCUSSION

In a remarkable feat of scientific ingenuity vaccines were developed within weeks after SARS-CoV-2 was identified as the etiological agent of COVID-19. This was facilitated by earlier outbreaks with related viruses such as SARS-CoV-1 in 2002-2004 or Middle East respiratory syndrome-related coronavirus (MERS-CoV), which appeared in 2012 in Saudi Arabia and has been circulating at low levels ever since Experimental vaccines had been developed and tested pre-clinically against these coronaviruses and provided blueprints for the COVID-19 vaccines based on RNAs or Ad vectors.

RNA vaccines, which were shown to be extraordinarily efficacious, have several disadvantages over Ad vector vaccines. RNA vaccines are in general safe but rare serious adverse events in form of anaphylactic reactions or myocarditis have been reported. They are heat labile and must be stored at around −80° C. and they are costly. Also, it appears that RNA-vaccine-induced S protein-specific antibody levels decline markedly after 6 months leading to reduced vaccine efficacy. This is supported by a previous trial testing an RNA vaccine against rabies virus, which showed that transgene product-specific antibody responses were not sustained.

Ad vector vaccines are heat stable and can be stored at 4° C. They are relatively inexpensive, which further facilitates their use in resource poor countries. They are very potent inducers of polyfunctional CD8⁺ T cell responses, which do not afford sterilizing immunity but provide a solid second layer of defense against viral spread within an organism. The efficacies of single-dose Ad vector vaccines by J&J or the single-vector, two-dose regimen by AstraZeneca's AdC vaccines are lower compared to the 2-dose RNA vaccine regimens while the Sputnik V vaccine from the Gamalaya Institute, which uses an HAdV26 vector prime followed by an HAdV5 boost has comparable efficacy, suggesting that a 2^{nd} immunization is essential for optimal efficacy and that a regimen using heterologous Ad vectors for two sequential immunizations might outperform an Ad vector-based vaccine regimen that gives the same vector twice. In general, Ad vectors are well tolerated although rare cases of potentially fatal cerebral venous sinus thrombosis (CVST) with thrombocytopenia have been linked to the J&J and AstraZeneca COVID-19 vaccines but not to the Sputnik V vaccines or to natural infections with Ad viruses. Such infections are common while CVST is very rare in the general population, suggesting contaminants rather than the Ad vector backbone or the expressed S protein as probable cause for CVST.

Although limited information is available on response longevity following Ad vector immunization, available data from pre-clinical models show that antibody titers are maintained for long periods of time while early clinical data suggest that antibody responses to SARS-CoV-2 induced by a 2 dose Ad vector vaccine decline less rapidly that those induced by RNA vaccines. This is likely linked to the biology of Ad vectors, which allows them to persist for extended periods of time mainly in lymphatic cells, thus maintaining active immune responses and delaying formation of memory, which also explains the needed for lengthy time intervals between two immunizations.

One clear disadvantage of Ad vector vaccines is that their immunogenicity is dampened by pre-existing VNAs to the vaccine carrier. Such antibodies can be induced by natural infections and consequently prevalence rates of VNAs to HAdV5 are globally high while VNAs to HAdV26 are common in humans residing in Africa. A single immunization with an Ad vector vaccine likely induces more modest levels due to lower antigenic loads although repeated vaccination with the same Ad vectors is expected to gradually increase titers so that they will reach those seen after a natural infection. One clinical study with an HAdV5-based COVID vaccine from CanSino Biologicals monitored induction of Ad vector-specific VNAs and, as expected, showed that they increased after vaccination. The same study reported an albeit insignificant inverse correlation between VNA titers to the HAdV5 vector and COVID S protein-specific antibody responses. VNAs to the vaccine vectors will likely pose limitations to repeated booster immunization using the same Ad vector by not only reducing, but also modifying transgene product-specific immune responses.

To avoid interference by Ad vector-specific VNAs, a vaccine regimen composed of two serologically distinct E1-deleted AdC vectors was developed. AdC vectors were selected as pre-existing VNAs to these chimpanzee viruses are rare in humans and individuals who have VNAs in general have low titers. Two serologically distinct AdC vector vaccines were developed to prevent blunting of booster immunizations by VNAs induced by the AdC vector used for priming. The two AdC vectors express the S protein of an isolate from Sweden, which is identical to that of the original Wuhan virus. The S sequence was not codon-optimized, nor were the K986P and V987P stabilizing mutations incorporated into the S2 sequence which both were used for the S gene expressed by the RNA vaccines or mutations of the S1/S2 furin cleavage site which, in addition to the stabilizing mutations, were incorporated into the S gene carried by the J&J COVID-19 vaccine. The Ad vectors, like those of J&J and the Gamalaya Institute, uses the original signal sequence of the S protein while the AstraZeneca vaccine uses an engineered leader sequence. Phase III trial results of the different Ad vector vaccines fail to indicate that any of these modifications have a major impact on vaccine efficacy.

The magnitude of S-specific antibody responses after a single immunization increased with higher vaccine doses and there were no significant differences between responses induced by the AdC6-S or AdC7-S vectors. This pattern changed after boosting, where the highest dose performed poorly. Timing of boosting seems to affect the magnitude of the recall response as was reported for both the AstraZeneca and J&J vaccines. IA short interval of 2 weeks between two vaccine doses resulted only in marginal increases in antibody titers as compared to a longer interval of 8 weeks. The optimal interval between repeated immunizations may depend on the vector dose used for priming with lower doses allowing for shorter intervals and may, well exceed 8 weeks for higher doses. Repeated use of the same vector also reduces the effectiveness of the booster immunization, which is likely caused by Ad vector specific VNAs, which reduce transduction rates and thereby expression levels of the transgene product. Again, the inhibitory effect of Ad vector-specific VNAs will depend on pre-existing memory and vector dose, which both affect the magnitude of the response, as well as on timing of the booster immunization with longer intervals being advantageous by allowing for a decline in Ad vector-specific antibody titers. Alternatively, inhibition by Ad vector specific VNAs can be circumvented by using heterologous vectors for prime boosting such as in the Sputnik V vaccine or the pre-clinical vaccines disclosed herein or by changing vaccine platforms. In the study, the importance of a boost was stressed by the relative lack of cross-reactivity of antibodies generated after a single immunization against S proteins with RBD mutations present in key variants such as the delta variant that has become dominant in many countries. Cross-reactivity markedly improved after a boost confirming previous clinical trial data. This could only in part be explained by increases in titers and may also reflect additional affinity maturation of the antibodies towards more conserved sequences of the RBD of the S protein.

In summary, and without wishing to be bound by theory, the results disclosed herein show that two serologically distinct AdC vectors expressing S of an early SARS-Co-V2 isolate induce robust and sustained antibody responses that cross-neutralize viral variants.

### Example 7: Prime-Boost Vaccinations with Two Serologically Distinct Chimpanzee Adenovirus Vectors Expressing SARS-CoV-2 Spike or Nucleocapsid Tested in a Hamster COVID-19 Model

Two serologically distinct replication-defective chimpanzee-origin adenovirus (Ad) vectors (AC) called AdC6 and AdC7 expressing the spike (S) or nucleocapsid (N) proteins of an early SARS-CoV-2 isolate were tested individually or as a mixture in a hamster COVID-19 challenge model. The N protein, which was expressed as a fusion protein within herpes simplex virus glycoprotein D (gD) stimulated antibodies and CD8+ T cells. The S protein expressing AdC (AdC-S) vectors induced antibodies including those with neutralizing activity that in part cross-reacted with viral variants. Hamsters vaccinated with the AdC-S vectors were protected against serious disease and showed accelerated recovery upon SARS-CoV-2 challenge. Protection was enhanced if AdC-S vectors were given together with the AdC vaccines that expressed the gDN fusion protein (AdC-gDN). In contrast hamsters that just received the AdC-gDN vaccines showed only marginal lessening of symptoms compared to control animals. These results indicate that immune response to the N protein that is less variable that the S protein may potentiate and prolong protection achieved by the currently used genetic COVID-19 vaccines.

### Cell Lines

HEK 293 cells were grown in Dulbecco's Modified Eagles medium (DMEM) supplemented with 10% fetal bovine serum (FBS) and antibiotics. BHK-21/WI-2 cells were grown in DMEM supplemented with 5% FBS and antibiotics. A549-hACE-TMPRSS2 (A549A/T) cells (InvivoGen, cat no. a549-hace2tpsa) were maintained in DMEM with 10% FBS, 300 μg/nl of hygromycin, 0.5 μg/ml of puromycin and antibiotics. All cells were maintained at 37° C. in in a 5% CO, incubator.

### Challenge Virus

Challenge was conducted with the WT WAS stock, which was generated from seed stock (2019-nCoV/USA-WA1/2020), obtained from Biodefense and Emerging Infections Research (BEI) resources (Cat #NR-52281, Lot #70036318) and expanded in Calu-3 cells at 37° C. for 3 days. The stock (Lot T 12152020-1235) titers in Vero TMPRSS2 cells are 2.34×10⁹ Median Tissue Culture Infectious Dose (TCID50)/mL and 3.68×10⁷ plaque forming units (PFU)/mL, and 6×10⁵ PFU/mL in Vero76 cells.

### Generation and Quality Control of AdC Vectors

Construction and expansion of the AdC-S and AdC-gDN vectors has been described (Zhou, et al., Nat Protoc. 2010; 5:1775-1785). Briefly the AdC-S vectors carry the gene encoding the full-length S gene (GenBank: QIC53204.1) while the AdC-gDN vectors carry the N gene (GenBank: QIC50514) that upon removal of the start and stop codons had been cloned into herpes simplex virus gD. Quality control assays for the AdC vector batches used for the hamster studies included titrations for vp content and infectious units, testing for sterility and endotoxin content and restriction enzyme digest of viral DNA, the latter to establish genetic integrity. The vectors passed the quality controls.

### Generation of VSV-S Vectors

VSV vectors pseudotyped with S of SARS-CoV-2 were generated in BHK-21/WT-2 cells using a the ΔG-GFP (G*ΔG-GFP) rVSV kit (Kerafast, Boston, MA, USA) and S sequences cloned into an expression plasmid under the control of the CMV promoter as described previously (Whitt, et al., J. Virol Methods. 2010; 169:365-74). One carries the same S protein as the vaccine vectors (VSV-SSWE). In a second the SSWE sequence was modified to incorporate the E484K and N501Y RBD mutations of the B1.351 South African variant (VSV-SSWE/B1.351). Others express either the S protein with N501Y and P681H RBD mutations present in the B1.1.7 UK variant (VSV-SSWE/B1.1.7), the L452R and D614G mutations of the Indian B.1.617.2 delta variant (VSV-S SWE/B.1.617.2), or a full-length synthetic sequence of the omicron variant BA.1/B.1.1.529.1 (VSV-S B.1.1.529.1).

### Hamster Challenge

Prior to challenge, hamsters were anesthetized with 80 mg/kg Ketamine and 5 mg/kg xylazine given i.m. The animals were challenged with 6×10³ PFU (Vero76 cells) in 100 μL per animal (50 p1/nostril). Administration of virus was conducted as follows: Using a calibrated P200 pipettor, 50 μl of the viral inoculum was administered dropwise into each nostril. Anesthetized animals were held upright such that the nostrils of the hamsters were pointing towards the ceiling. The tip of the pipette was placed into the first nostril and virus inoculum was slowly injected into the nasal passage, and then removed. This was repeated for the second nostril. The animal's head was tilted back for about 20 seconds and then the animal was returned to its housing unit. Approximately 20 min post challenged, antisedan (0.04 ml, 1 mg/kg) was given i.m. to all hamsters.

### Antibody Assays

### ELISA for Anti-S1 and Anti-S2 Antibodies

Sera from individual hamsters were tested for S-specific antibodies by ELISA on plates coated overnight with 100 μl of a mixture of S1 and S2 (Native Antigen Company, Kidlington, UK) each diluted to 1 μg/ml in bicarbonate buffer. The next day plates were washed and blocked for 24 hours at 4° C. with 150 μl of a 3% BSA-PBS solution. Sera were diluted in 3% BSA-PBS and 80 μl of the dilutions were added to the wells after the blocking solution had been discarded. Plates were incubated at room temperature for 1 hour and then washed 4× with 150 μl of PBS. An anti-hamster IgG (H+L)-Alkaline Phosphatase antibody produced in goat (Sigma-Aldrich. St Louis, MO) diluted to 1:1000 in 3% BAS-PBS was added at 60 μl/well for 1 hour at room temperature Plates were washed 4 times and 100 μl of substrate composed of phosphatase substrate tables (Sigma-Aldrich, St Louis, MO) diluted in 5 ml of diethanolamine buffer per tablet was added. Plates were read in an ELISA reader at 405 nm. Coated wells only receiving the substrate served to determine background. Background data were subtracted from the experimental data. Data are expressed as area under the curve for the different dilutions.

### ELISA for RBD-Binding Antibodies

Sera were tested for inhibition of ACE binding to the RBD of the S protein by the Anti-SARS-CoV-2 Neutralizing Antibody Titer Serologic Assay Kit from Acro Biosystems (Newark, DE) following the manufacturer's instructions. A positive standard with a known concentration provided by the kit was used to extrapolated anti-S antibody concentrations into μg per ml of serum.

Neutralization Assay with S Protein Pseudotyped VSV Vectors

VSV-S vector was initially titrated on A549A/T cells. Cells were diluted to 4×10⁵ cells/ml in DMEM with 10% FBS and 5 μl of the cell dilution were added to each wells of Terasaki plates. The next day, serial dilutions of VSV-S vectors were added to duplicate wells. Numbers of fluorescent cells/well were counted with a fluorescent microscope 48 hours later.

For the neutralization assay, a dose of VSV-S vector that infected ˜20-40 cells/well was selected. A549A/T cells were plated into Terasaki plate wells as described above. The following day, sera were serially diluted in DMEM with 10% FBS. The VSV-S vector was diluted in medium containing 1 μg/ml of a mouse monoclonal antibody against VSV glycoprotein (sc-365019, Santa Crusz Biotechnology, Dallas, TX) and then incubated with the serum dilutions for 90 min at room temperature under gentle agitation starting with a serum dilution of 1 in 40. 5 μl aliquots of the mixtures were transferred onto Terasaki plate wells with A549A/T cells. Each serum dilution was tested in duplicates, an additional 4-6 wells were treated with VSV-S that had been incubated with medium rather than serum. Plates were incubated for 48 hours and then numbers of fluorescent cells were counted. Titers were set as the last serum dilution that reduced numbers of green-fluorescent cells by at least 50%.

### ELISA for N Protein-Specific Antibodies

Sera were tested for antibodies to the N protein by an ELISA on plates coated with 1 μg of a purified N protein (Abbexa, abx163973) using the same procedures as for antibodies to the S protein. A monoclonal antibody to N protein (Abbexa, MCA6373, lot #156962) served as a positive control (data not shown).

### T Cell Assays

Blood diluted in EDTA was collected 2 weeks after the 2nd immunization and delivered within 6 hours after collection to the laboratory. PBMCs were purified by Ficoll) Paque Plus (GE Healthcare, Chicago, IL) gradient centrifugation for 30 min at 2800 rpm. Cells were washed and seeded into 96-roundbottom well plates (0.2-1×10⁶ cells per well). Lymphocytes were stimulated with three pools of peptides representing the N sequence present in the vaccines. Peptides were 15 amino acids in length and overlapped by 10 amino acids with the adjacent peptides. Individual peptides were diluted according to the manufacturer's instructions. For stimulation ˜10⁶ lymphocytes plated in medium containing 2% fetal calf serum and 1.5 μl/ml Golgiplug (BD Bioscience; San Jose, CA) were cultured with peptides each present at a final concentration of 2 μg/ml for 5 hr at 37° C. in a 5% C02 incubator. Control cells were cultured without peptides. Following stimulation, cells were incubated with anti-CD8b-PE (clone eBio341, eBioscience 53-6.7 and violet live/dead dye (Thermo Fisher Scientific) at 4° C. for 30 min in the dark. Cells were washed once with PBS and then fixed and permeabilized with Cytofix/Cytoperm (BD Biosciences, San Jose, CA) for 20 min. Cells were then incubated with an anti-INF-7-FITC antibody (clone XMG1.2 BioLegend. San Diego, CA) at 4° C. for 30 min in the dark. Cells were washed and fixed in 1:3 dilution of BD Cytofix fixation buffer (BD Pharmingen, San Diego CA). They were analyzed by a BD FACS Celesta (BD Biosciences, San Jose, CA) and DiVa software. Post-acquisition analyses were performed with FlowJo (TreeStar, Ashland. OR). Data shown in graph represents % of INF-γ production by CD8+ cells upon peptide stimulation. Background values obtained for the same cells cultured without peptide(s) were subtracted.

Genomic mRNA PCR Assay

The qRT-PCR assay utilizes primers and a probe specifically designed to bind to a conserved region of N gene of SARS-CoV-2. The signal is compared to a known standard curve and calculated to give copies per mL. For the qRT-PCR assay, viral RNA was first isolated from oral swab using the Qiagen MinElute virus spin kit (cat. no. 57704). For tissues it was extracted with RNA-STAT 60 (Tel-test “B”)/chloroform, precipitated and resuspended in RNAse-free water. To generate a control for the amplification reaction, RNA was isolated from the applicable SARS-CoV-2 stock using the same procedure. The amount of RNA was determined from an O.D. reading at 260, using the estimate that 1.0 OD at A260 equals 40 μg/mL of RNA. With the number of bases known and the average base of RNA weighing 340.5 g/mole, the number of copies was then calculated, and the control diluted accordingly. A final dilution of 10⁸ copies per 3 μl was then divided into single use aliquots of 10 μl. For the master mix preparation, 2.5 mL of 2× buffer containing Taq-polymerase, obtained from the TaqMan RT-PCR kit (Bioline cat #BIO-78005), was added to a 15 mL tube. From the kit, 50 μl of the RT and 100 μl of RNAse inhibitor was added. The primer pair at 2 μM concentration was then added in a volume of 1.5 ml. Lastly, 0.5 ml of water and 350 μl of the probe at a concentration of 2 μM were added and the tube vortexed. For the reactions, 45 μl of the master mix and 5 μl of the sample RNA was added to the wells of a 96-well plate. The plates were sealed with a plastic sheet. All samples were tested in triplicate.

For control curve preparation, samples of the control RNA were prepared to contain 10⁶ to 10⁷ copies per 3 μL. Eight (8) 10-fold serial dilutions of control RNA was prepared using RNAse-free water by adding 5 μl of the control to 45 μl of water and repeating this for 7 dilutions resulting in a standard curve with a range of 1 to 10⁷ copies/reaction. Duplicate samples of each dilution were prepared as described above. If the copy number exceeded the upper detection limit, the sample was diluted as needed. For amplification, the plate was placed in an Applied Biosystems 7500 Sequence detector and amplified using the following program: 48° C. for 30 minutes, 95° C. for 10 minutes followed by 40 cycles of 95° C. for 15 seconds, and 1 minute at 55° C. The number of copies of RNA per ml was calculated by extrapolation from the standard curve and multiplied by the reciprocal of 0.2 ml extraction volume. This gave a practical range of 50 to 5×10⁸ RNA copies per gram tissue.

Primers/probe sequences:
2019-nCoV_N1-F:
(SEQ ID NO: 109)
5′-GAC CCC AAA ATC AGC GAA AT-3′
2019-nCoV_N1-R:
(SEQ ID NO: 110)
5′-TCT GGT TAC TGC CAG TTG AAT CTG-3′
2019-nCoV_N1-P:
(SEQ ID NO: 111)
5′-FAM-ACC CCG CAT TAC GTT TGG TGG ACC-BHQ1-3′.

Sub-Genomic mRNA Assay

For the qRT-PCR assay, viral RNA is first isolated using the Qiagen MinElute virus spin kit (cat. no. 57704). For tissues, the tissue was homogenized and RNA is extracted with RNA-STAT 60 (Tel-test “B”)/chloroform, precipitated and resuspended in AVE Buffer (Qiagen 1020953). The qRT-PCR assay utilizes primers and a probe specifically designed to amplify and bind to a conserved region of N gene of SARS-CoV-2. The signal was compared to a known standard curve and calculated to give copies per ml. To generate a control for the amplification reaction, RNA was isolated from the applicable virus stock using the same procedure. The amount of viral RNA was determined comparing it to a known quantity of plasmid control. A final dilution of 10P copies per 3 μl was then divided into single use aliquots of 10 μl. For the master mix preparation, 2.5 ml of 2× buffer containing Taq-polymerase, obtained from the TaqMan RT-PCR kit (Bioline #BIO-78005), was added to a 15 ml tube. From the kit, 50 μl of the RT and 100 μl of RNAse inhibitor was also added. The primer pair at 2 μM concentration was then added in a volume of 1.5 ml. Lastly. 0.5 ml of water and 350 μl of the probe at a concentration of 2 μM are added and the tube vortexed. For the reactions, 45 μl of the master mix and 5 μl of the sample RNA were added to the wells of a 96-well plate. The plates were sealed with a plastic sheet. All samples were tested in triplicate. For control curve preparation, the control viral RNA was prepared to contain 10⁶ to 10⁷ copies per 3 μl. Seven 10-fold serial dilutions of control RNA were prepared using RNAse-free water by adding 5 μl of the control to 45 μl of water and repeating this for 7 dilutions. This gives a standard curve with a range of 1 to 10⁷ copies/reaction. The sub-genomic-N uses a known plasmid for its curve. Duplicate samples of each dilution were prepared as described above. If the copy number exceeded the upper detection limit, the sample was diluted as needed. For amplification, the plate was placed in an Applied Biosystems 7500 Sequence detector and amplified using the following program: 48° C. for 30 minutes. 95° C. for 10 minutes followed by 40 cycles of 95° C. for 15 seconds, and 1 minute at 55° C. The number of copies of RNA per ml is calculated by extrapolation from the standard curve and multiplying by the reciprocal of 0.2 ml extraction volume. This gives a practical range of 50 to 5×10⁸ RNA copies per mL for oral swabs and BAL, and for lung tissue the viral loads are given per gram.

Primers/probe sequences:
sg-N-F:
(SEQ ID NO: 112)
5′-CGATCTCTTGTAGATCTGTTCTC-3′
sg-N-R:
(SEQ ID NO: 113)
5′-GGTGAACCAAGACGCAGTAT-3′
Sg-N-P:
(SEQ ID NO: 114)
5′-6-FAM/TAACCAGAA/ZEN/TGGAGAACGCAGTGGG/3IABKFQ/

### Lung Histology

Parts of the lungs were placed in 10% neutral buffered formalin for histopathologic analysis. Lung was processed to hematoxylin and eosin (H&E) stained slides and examined by a board-certified pathologist at Experimental Pathology Laboratories, Inc. (EPLX) in Sterling, Virginia. Findings were graded from one to five, depending upon severity.

### Statistical Analyses

Data were analyzed by 2-or 1-way Anova or Kruskal-Wallis test with Dunn correction for multiple comparisons. Correlations were carried out by one-tailed Spearman's rank correlation tests. P values equal or less than 0.05 were considered significant. The analyses were carried out by GraphPad Prism.

### Results

### Experimental Design

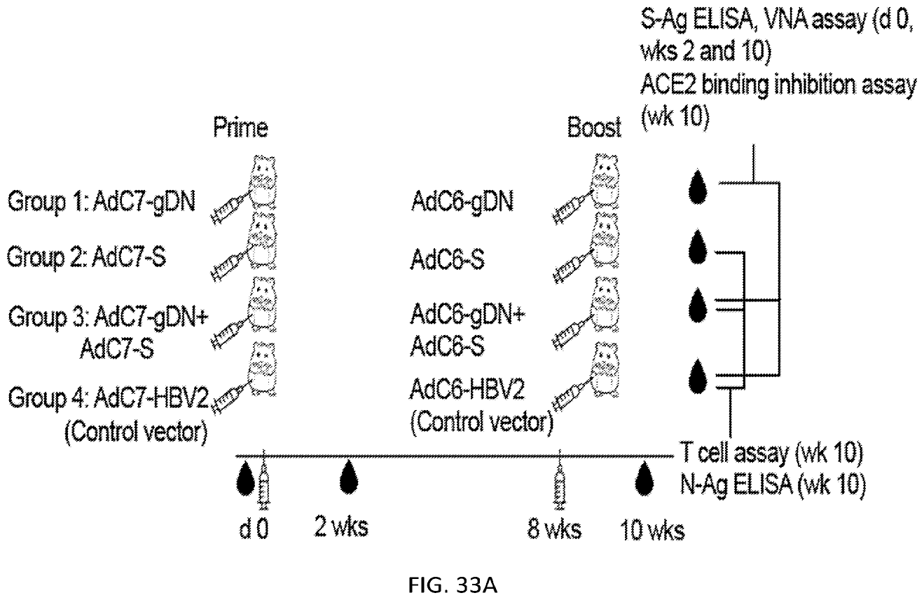

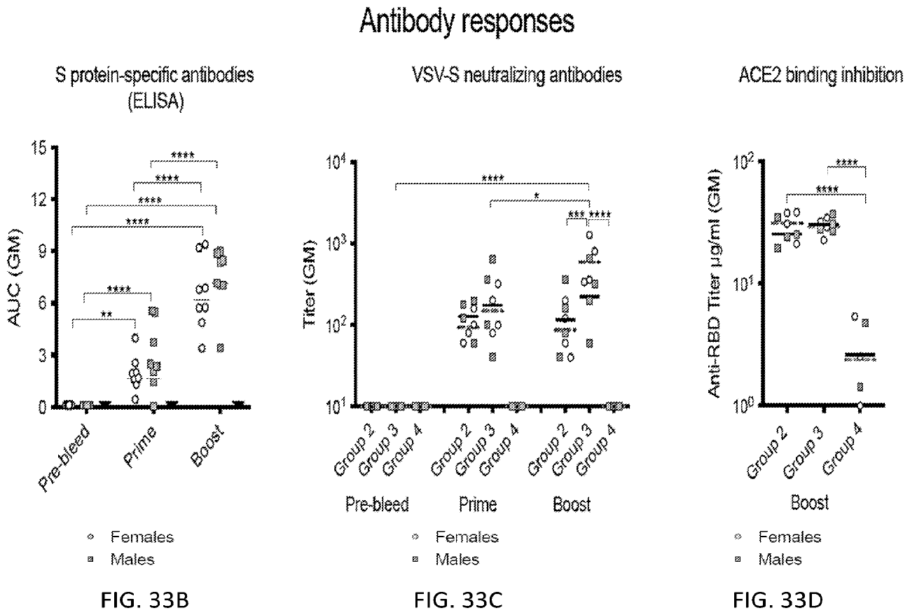

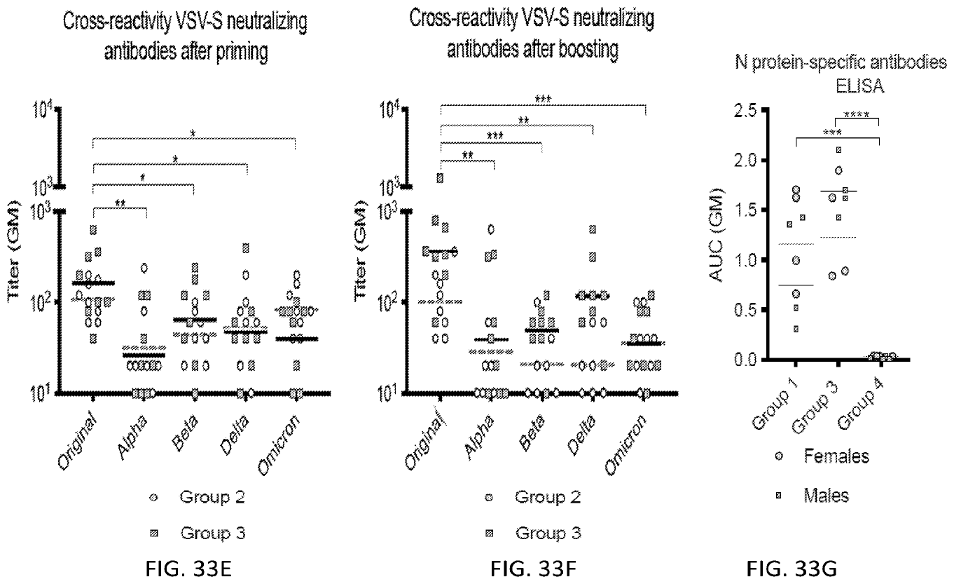

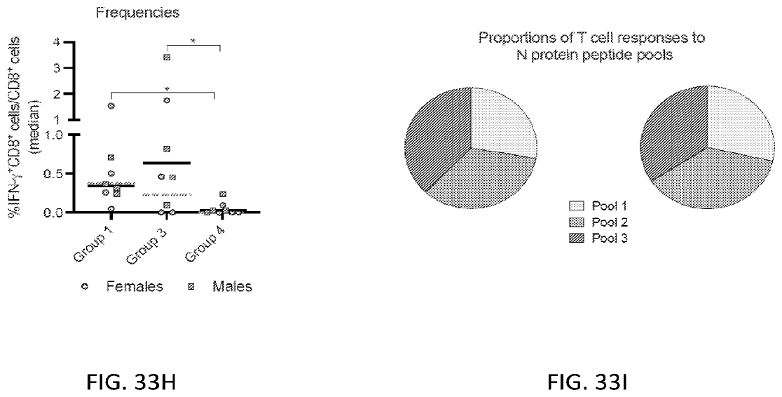

Sixteen female and sixteen male Syrian golden hamsters were enrolled and separated into 4 groups of 8 animals each (4 females, 4 males/group). The groups were primed by intramuscular (i.m.) injections as follows: group 1 was injected with 1×10¹⁰ virus particle (vp) of AdC7-gDN, group 2 with 1×10¹⁰ vp of AdC7-S, group 3 with a mixture of 0.5×10¹⁰ vp AdC7-gDN and 0.5×10¹⁰ AdC6-S and group 4 with 1×10¹⁰ vp of the control vector, called AdC7-HBV2, which expresses a sequence derived from hepatitis B virus. Only half of the vaccine dose was used for each component of the mixture to keep the total amount of vector given to each animal constant, as in a clinical setting higher vaccine doses translate to increases in adverse events driven by innate responses to the Ad vector. Animals were boosted 2 months after priming with AdC6 vectors expressing the same inserts and used at the same doses. Hamsters were bled at baseline, 14 days after the prime and 14 days after the boost to determine SARS-CoV-2 S protein-specific antibody responses in groups 2-4. N-specific antibody and CD8+ T cell responses in groups 1, 3 and 4 were measured from blood 14 days after the boost (FIG. 33A). Animals were challenged intranasally with SARS-CoV-2 4 weeks after the boost. After challenge animals were checked daily for clinical symptoms and weight loss. Oral swaps were collected on days 2, 4, 7 and 14 after viral challenge to determine levels of viral genomic and sub-genomic (sg) RNA. Four animals in each group were euthanized 4 days and the others 14 days after challenge. After euthanasia, lung viral titers were determined, and lung sections were screened for pathology.

### Immune Responses to the Vaccine Vectors

Sera from hamsters of groups 2, 3 and 4 harvested at baseline and at 2 weeks after the prime or boost were tested by ELISA on plates coated with S1 and S2 proteins, which measures all S-binding antibodies (FIG. 33B). They were also tested in a neutralization assays using vesicular stomatitis virus (VSV) vectors pseudo-typed with SARS-CoV-2 S protein to detect VNAs, which can either be directed to the RBD sequence of S1 or the fusion peptide in S2 (FIG. 33C). Sera harvested 2 weeks after the boost were in addition tested by an ELISA, which measures antibodies specific to the receptor binding domain (RBD) of S1 (FIG. 33D). All pre-immunization samples and samples from the control groups harvested at either time point scored negative in either one of these assays. All but one of the sera from one hamster of groups 2 scored positive by the S1/S2-specific ELISA by 2 weeks after the prime. All hamsters seroconverted after the boost and overall responses showed significant increases (FIG. 33B). More specifically geometric mean (GM) titers of group 2 females increased 1.6-fold, while those of males increased 6.8 fold; GM titers of group 3 females increased 1.5 fold while those of males increased 1.9 fold. There were no significant differences in responses of males compared to females although in group 2 males tended to have higher responses after the boost than females. All hamsters developed antibodies that neutralized the pseudotyped VSV-S virus with GM titers of 109 in group 2 and 167 in group 3 after the prime which changed to 166 and 365 after the boost, respectively. The superior booster response in group 3 was driven by female hamsters, which showed a 4-fold increase in VNA titers compared to the marginal 1.2-fold increase in males. After the boost all animals scored positive in the RBD-specific ELISA (FIG. 33D) and there were no significant differences between males and females or groups 2 and 3.

VNAs were tested for reactivity against alpha, beta, delta, and omicron variants. Sera collected after the prime (FIG. 33E) or after the boost (FIG. 33F) showed significantly reduced titers comparing neutralization of VSV-S vectors expressing wild-type S protein to those with S proteins of variants.

Sera from hamsters of groups 1, 3 and 4 harvested after the boost were tested for antibodies to the N protein. All AdC-gDN vaccinated animals scored positive with no differences between groups 1 and 3 or males and females hamsters (FIG. 33G).

Peripheral blood lymphocytes were tested two weeks after the boost for production of IFN-γ by CD8+ T cells in response to three peptide pools representing the N protein sequence. Most animals of groups 1 and 3 developed N protein-specific CD8+ T cell responses; 1 and 2 females of groups 1 and 3, respectively and 1 male of group 3 failed to respond (FIG. 33H). Responses showed no preference to any of the 3 peptide pools representing different segments of the N protein (FIG. 33I).

Clinical Symptoms and Weigh Loss after SARS-CoV-2 Challenge

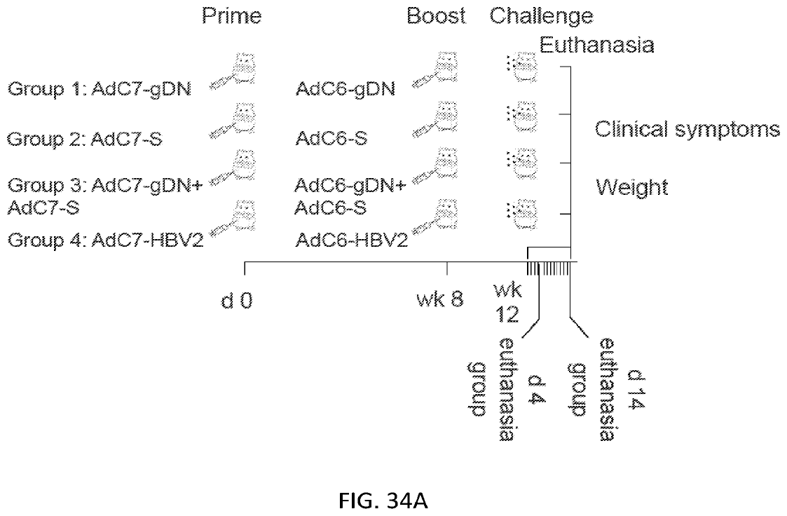

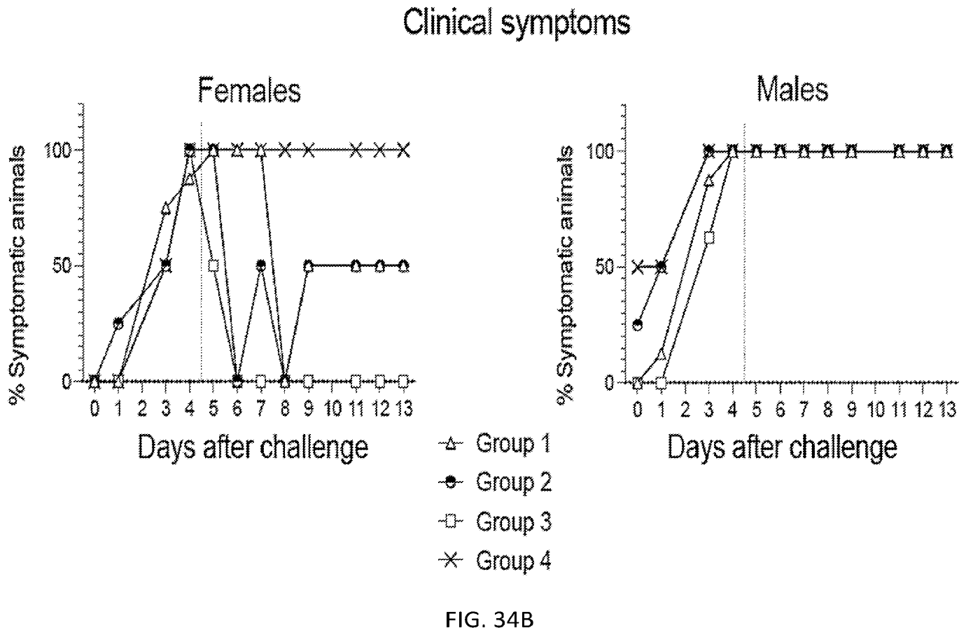

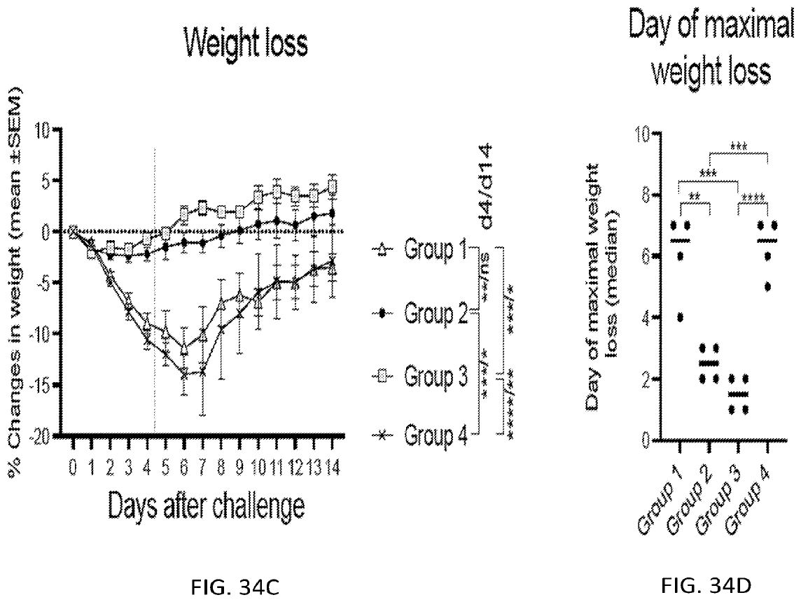

Four weeks after the boost animals were challenged intranasally with SARS-CoV-2 (FIG. 34A). Animals were scored twice daily for clinical symptoms. Most animals developed benign symptoms after challenge in form of mildly ruffled fur, hunched over posture and/or closed or squinted eyes. None of the vaccinated mice exceeded a COVID-19 disease score of 1 and only one female in the control group 4 was given a disease score of 2 on days 5-7 after challenge. One male animal in the control group died 7 days after challenge although it never exceeded a disease score of 1. His lung pathology suggested that his death was most likely caused by SARS-CoV-2.

Plotting days of clinical symptoms against days after challenge showed that most female animals developed symptoms by day 4 after challenge but for one group 3 animal, which became symptomatic on day 5. All males of group 4 and most of the males of the vaccine groups had symptoms by day 3 and by day 4 all male animals had visible signs of disease. Thereafter the disease course was markedly different for vaccinated female and male animals as has also been reported for humans. In both gender groups, unvaccinated hamsters exhibited symptoms till the day of their euthanasia. The same was seen for all males of the vaccine groups. With some fluctuations, half of the females of group 1 and 2 remained symptomatic throughout the observation period while group 3 females became and remained symptom-free by days 5 or 6 (FIG. 34B). Although this result is based on small numbers of animals, it nevertheless suggests that vaccinated females recover more rapidly than vaccinated males upon breakthrough infections and that the combination vaccine compared to the individual vaccines accelerated recovery in females.

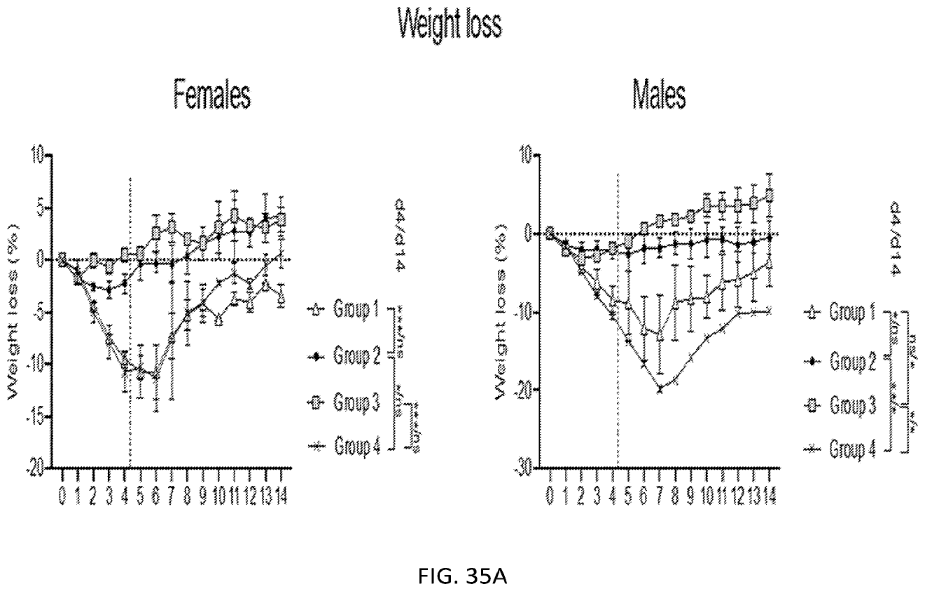

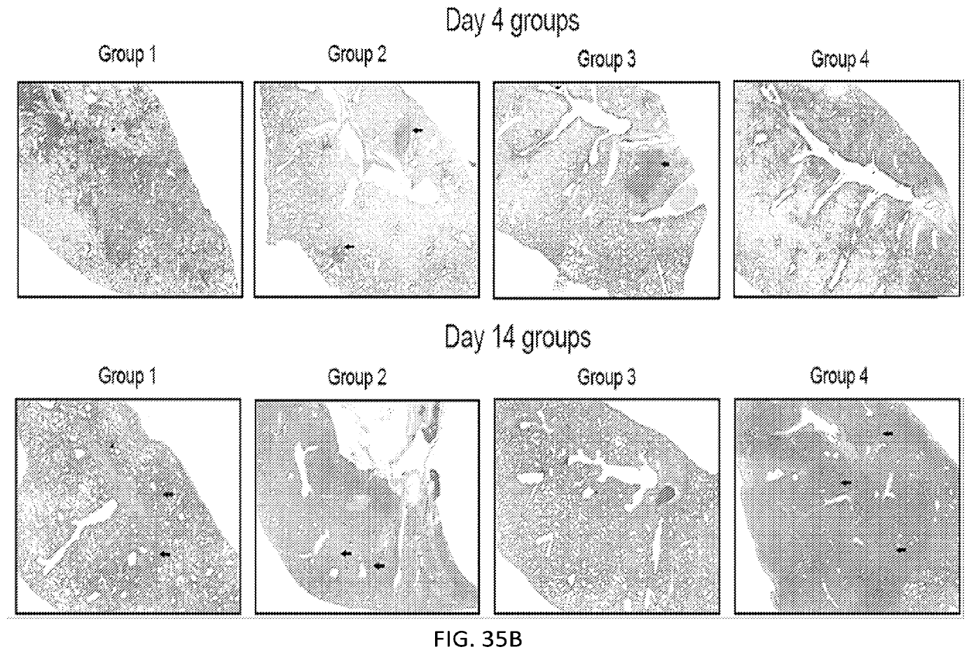

These data were mirrored by levels of weight loss. Control animals rapidly started losing weight after challenge (FIG. 34C) with maximal weight loss around days 6-7 (FIG. 34D). Group 1 animals which had only received AdC-gDN vectors lost weight with similar kinetics to the control group although their maximal weight loss by days 6 and 7 after infection tended to be lower. Hamsters of groups 2 and 3 lost only a minimal amount of weight early after challenge and started gaining weight by day 2 or 3 after challenge. On days 4 and 14 after challenge groups 2 and 3 showed significantly less weight loss compared to the control group 4: group 3 showed a significant difference to group 1 at both time points while group 2 only differed from group 1 by day 4 after infection. Females recovered their weight 1-2 days earlier than males (FIG. 35A).

### Effect of SARS-CoV-2 on Viral Loads and Lung Histology

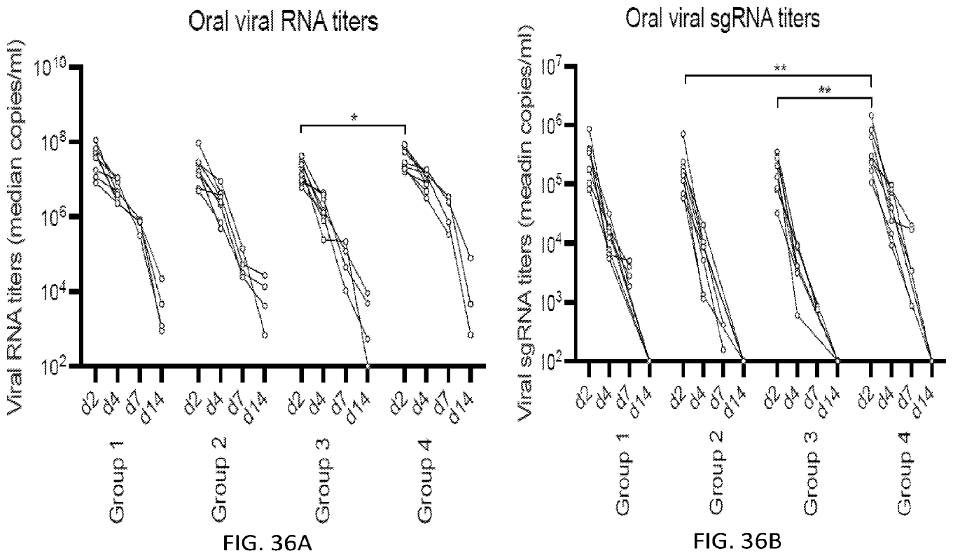

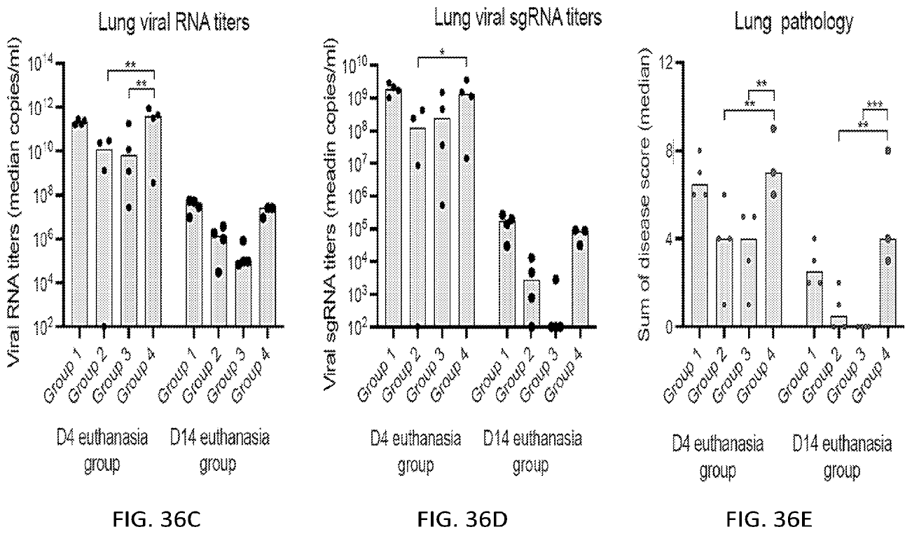

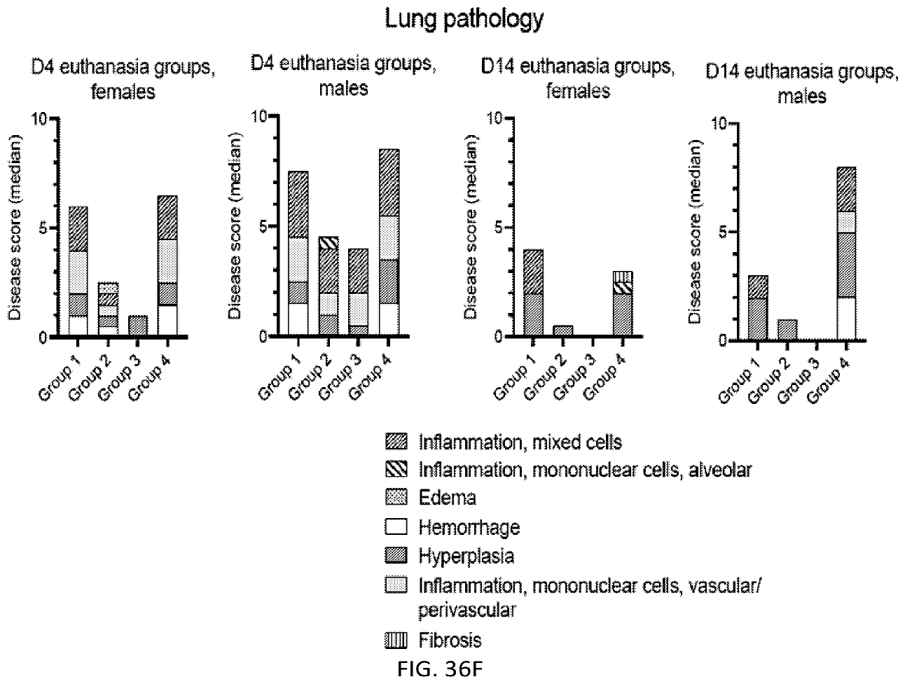

Viral titers were determined from oral swabs collected on days 2, 4 (8 animals for each group), 7 and 14 (4 animals for each group)(FIG. 36A) and lung tissues collected from 4 animals of each group either on days 4 or 14 after challenge. Samples were screened for viral RNA and sgRNA, the latter has been suggested to determine titers of infectious virus more accurately. Immunization with the vaccine mixture reduced oral viral RNA (FIG. 36A) and sgRNA (FIG. 36B) titers on day 2 after infection while immunization with the Ad-S vaccine only reduced sgRNA titers but not RNA titers on day 2. No significant differences were observed for the group 1 or the other time points. Vaccination with either the AdC-S or the AdC-S+AdC-gDN regimens reduced lung viral RNA titers (FIG. 36C) tested on day 4 after challenge while AdC-S vaccination also reduced sgRNA titers at that time point (FIG. 36D). By day 14, viral loads in lungs were markedly reduced in all groups and although group 3 continued to show lower RNA and sgRNA titers compared to the others this failed to reach significance.

Parts of the lungs were sectioned and stained after euthanasia for 4 animals of each group on day 4 and the remaining animals on day 14 after challenge and analyzed for pathological changes (FIG. 36E). Macroscopically lungs showed red or dark red patches, dark spotted, mottled/red lobes in groups 1, 2 and 4 but not 3 (FIG. 35B). Tissue lesions were graded microscopically and lungs from most animals showed grade 1-3 histopathology. Especially on day 4 lungs showed perivascular edema, alveolar hemorrhage, bronchiolo-alveolar hyperplasia, mixed or mononuclear cell inflammation (bronchoalveolar, alveolar, and/or interstitial), mononuclear vascular/perivascular inflammation, mesothelial hypertrophy, and/or pleural fibrosis (FIG. 36E and FIG. 36F). In groups 2 and 3 lesions were milder and more multifocal than in groups 1 and 4 on day 4. The sum of the score for the individual symptoms showed on day 4 significant differences between groups 2 and 3 compared to the control animals of group 4. On day 14 lungs of the control animals continued to show marked pathology while group 3 animal lungs no longer showed any lesions: group 2 animals showed residual mild disease. Group 1 animals, which only received the AdC-gDN vaccine, also showed reduced pathology compared to group 4 animals (FIG. 36E). Lung pathology showed gender specific differences (FIG. 36F). By day 4 after infection the two group 3 females only showed grade 1 hyperplasia while the 2 males of this group had additional lesions. By day 14 both females of the control group showed a lessening of symptoms, which was not observed in the surviving male.

### Correlations

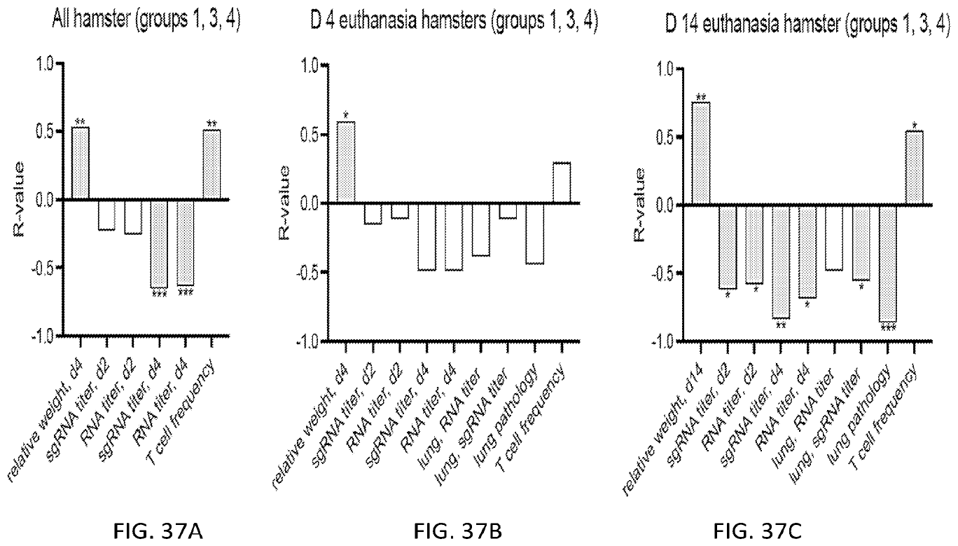

The data were analyzed for correlations by Spearman separately for hamsters that were tested for S protein specific antibody responses (groups 2 and 3) and/or N protein specific T and B cell responses (group 1, 3 and 4). T cell responses inversely correlated with oral RNA and sgRNA titers on day 2 (p=0.037/0.02) while S protein specific antibody by the S1/S2 ELISA after the boost showed inverse correlations for oral viral RNA or sgRNA titers on day 4 (ELISA: p=0.049/0.029). For other parameters such as relative weight after challenge, lung pathology or lung viral titers, animals that were euthanized on day 4 were analyzed separately from those that were euthanized on day 14, again keeping the T and B cell groups separate. These analyses showed, as expected, direct correlations between lung viral loads and lung pathology. Significant correlations were not observed between T cell responses and any of the parameters of disease. In the early euthanasia group, VNA titers after the boost inversely correlated with RNA and sgRNA lung viral titers on day 4 (RNA p=0.01, sgRNA p=0.006), while the late euthanasia group showed antibody responses by S1/S2 ELISA after the boost inversely correlated with oral viral RNA or sgRNA titers on day 4 (p=0.01 for both). The most pronounced correlations were seen for antibody responses to the N proteins in groups 1. 3 and 4. N protein specific antibody responses showed for the analyses with all animals and the day 4 or 14 euthanasia group animals strong positive correlations with relative weight and for the former two analyses strong inverse correlations with RNA and sgRNA titers in saliva on day 4. The day 14 euthanasia animals also showed strong inverse correlations between N protein specific antibody titers and sgRNA and RNA titers in saliva on days 2 and 4, lung sgRNA titers and lung pathology (FIGS. 37A-37C).

### Conservation of T and B Cell Epitopes

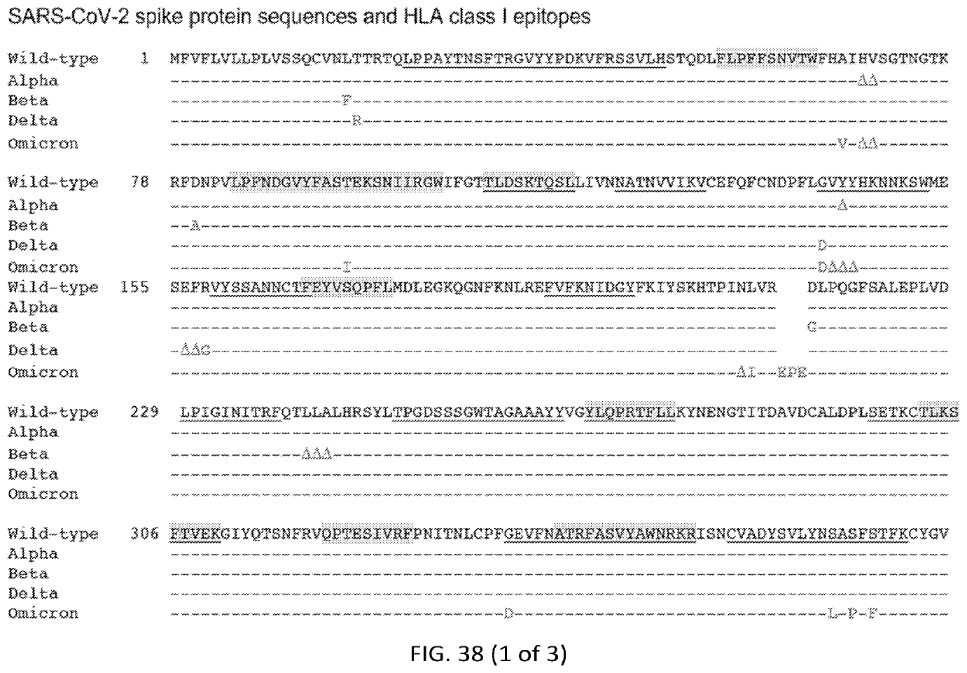

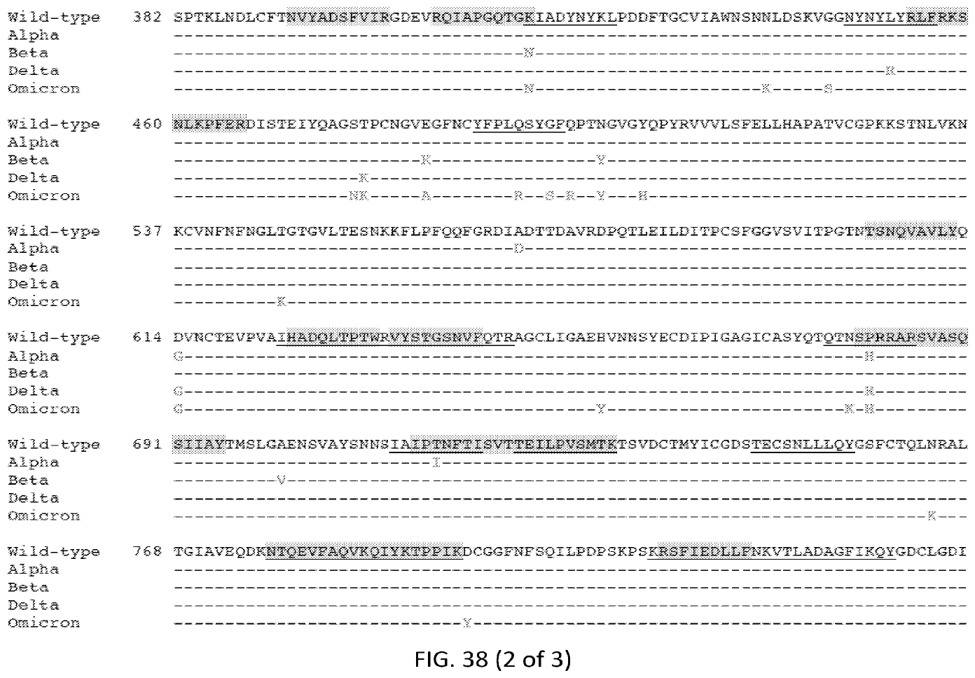

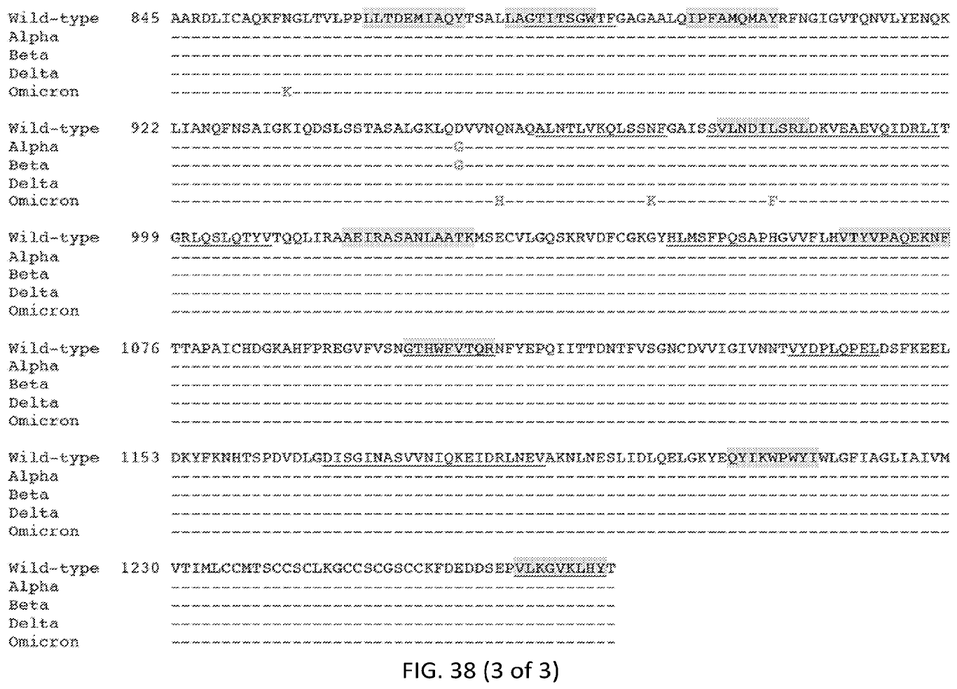

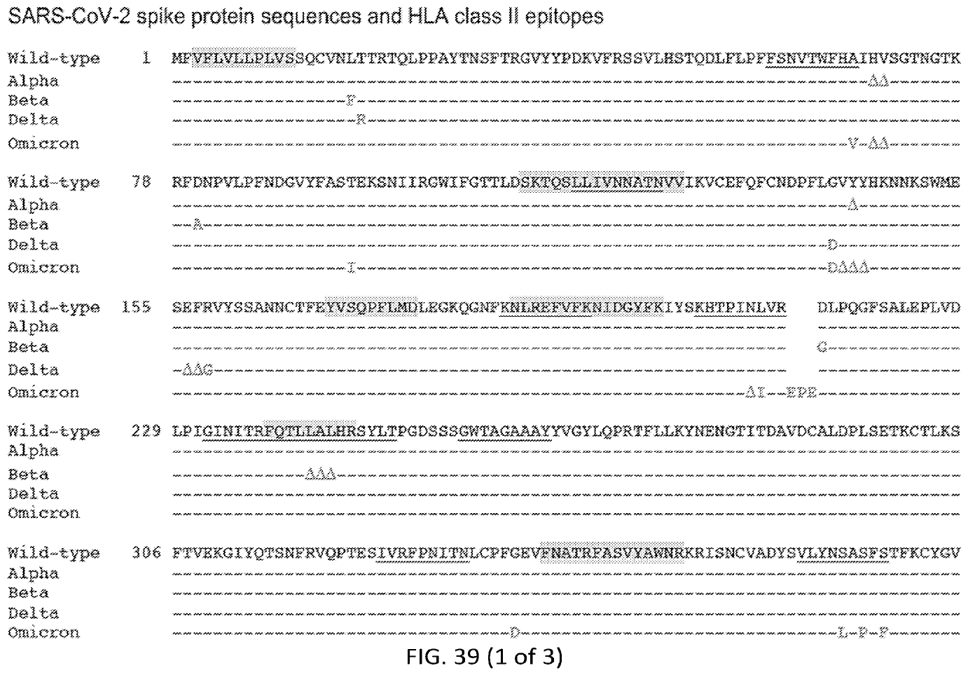

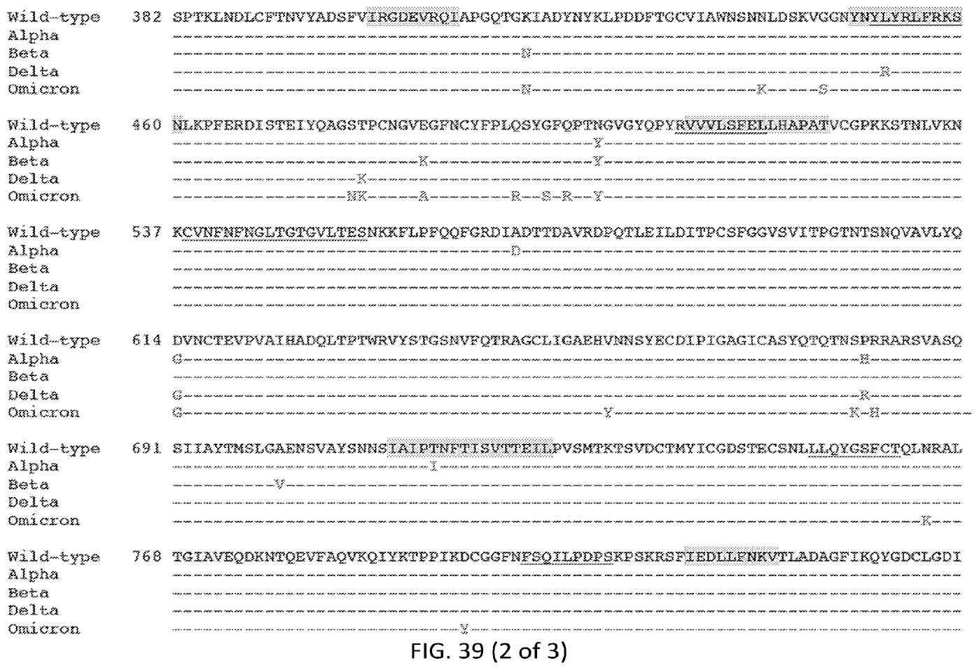

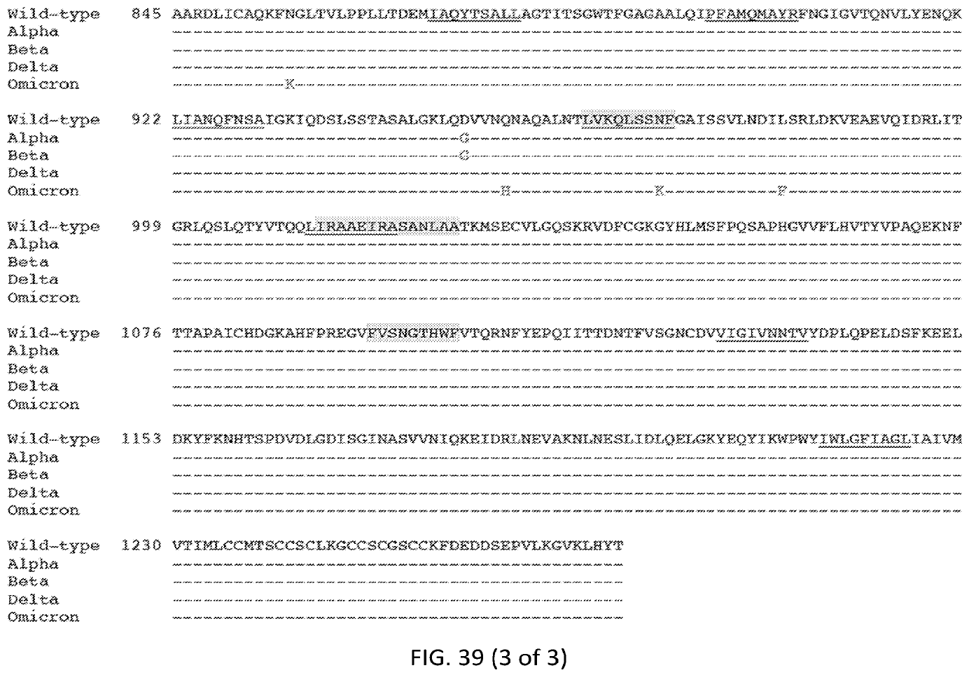

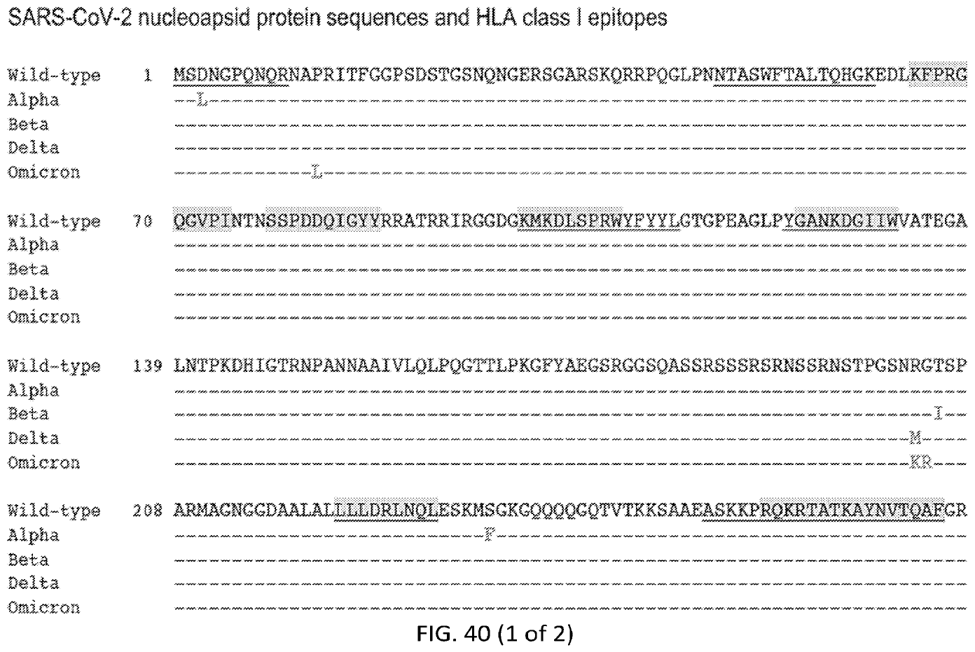

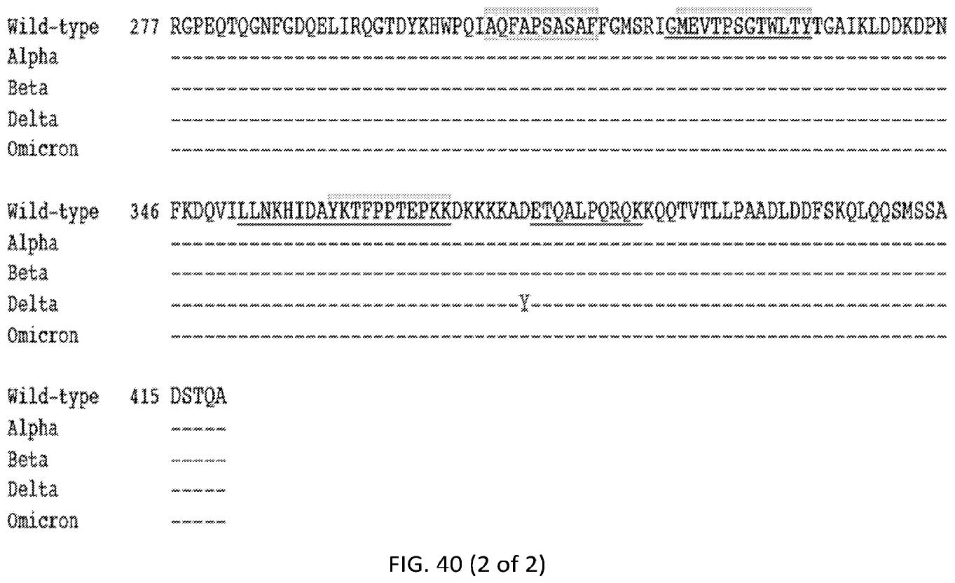

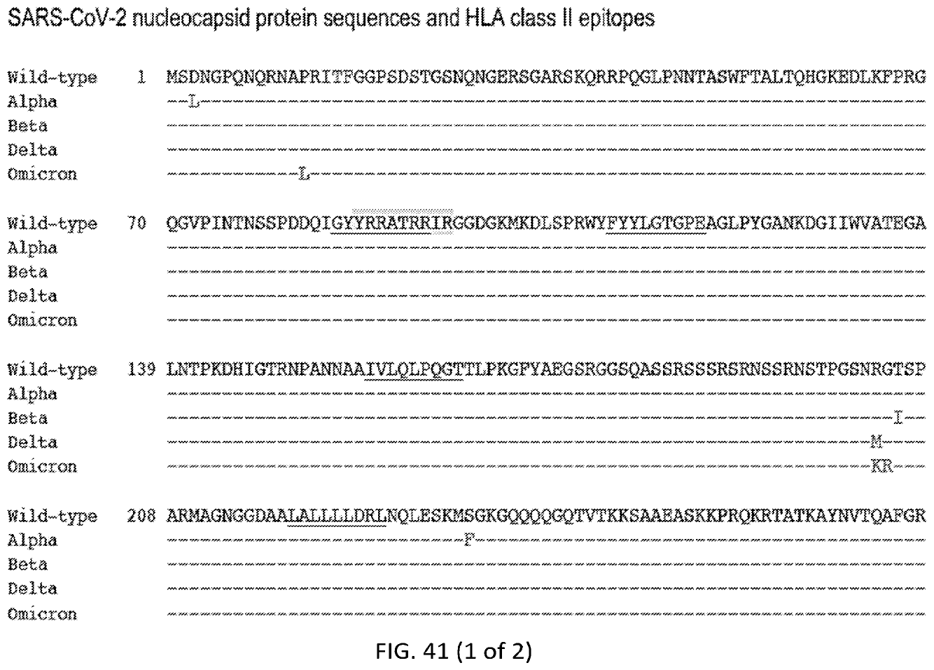

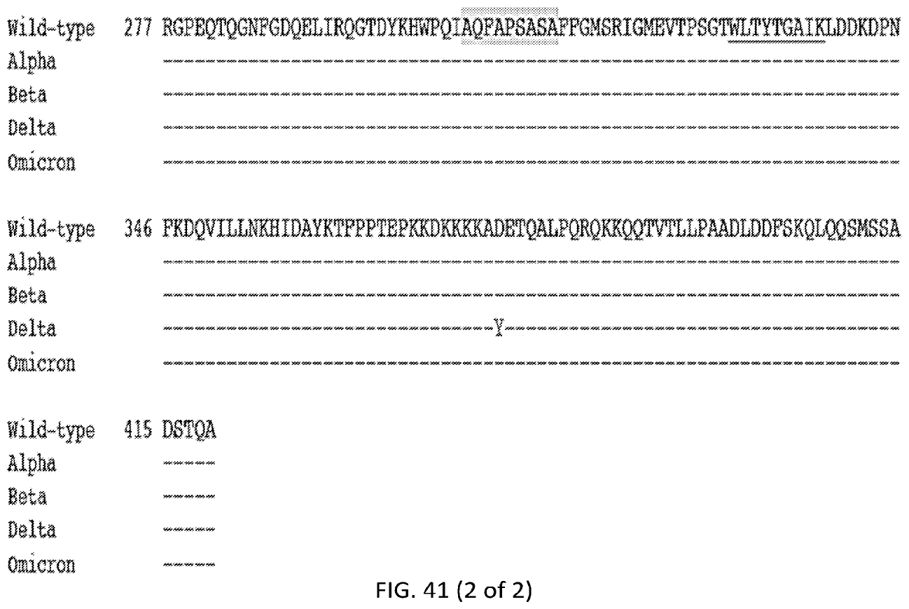

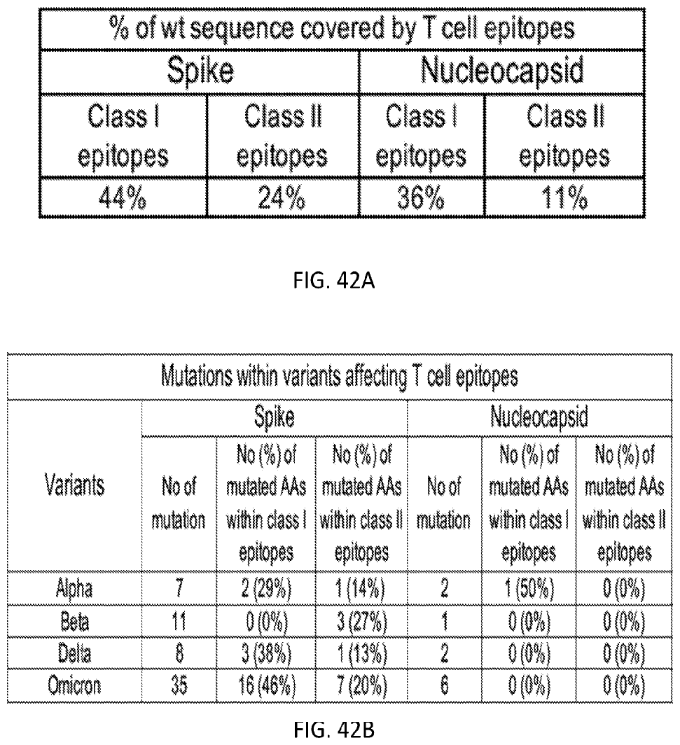

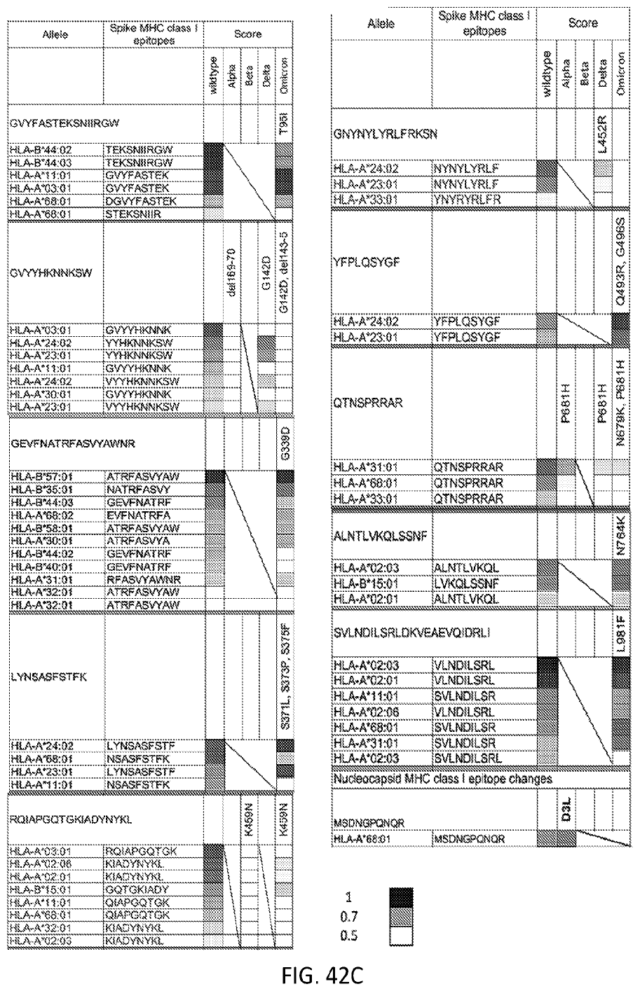

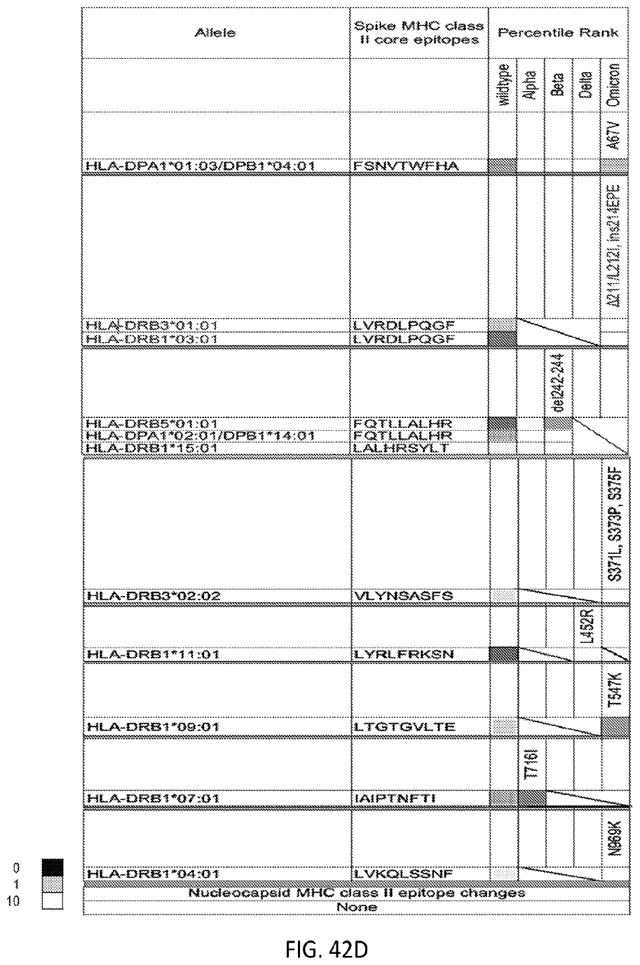

The results demonstrated herein show that vaccine induced S protein-specific immune responses reduce COVID-19 disease after SARS-CoV-2 infection and that addition of vaccines expressing the N protein has significant benefits. The S protein of SARS-CoV-2 has undergone extensive mutations, some of which increase viral transmission and affect neutralization by vaccine-induced antibodies. Both the S and N protein have numerous MHC class I and 11 epitopes for recognition by CD8+ and CD4+ T cells (FIGS. 38-41) and such sequences may be more conserved than those of VNA binding epitopes. Using epitope prediction software (http://tools.iedb.org/main/tcell/), HLA class I and II epitopes present within the SARS-CoV-2 S and N protein sequences, and how these epitopes have been affected by mutations within variants, were analyzed. For HLA class I epitopes, a cut-off score of 0.8 was used, while for HLA class II epitopes, a cut-off rank of ≤1 was used. The S protein is very rich in HLA class I epitopes and about 44% of the sequence can be recognized by CD8+ T cells from individuals with different HLAs while 24% of the sequence are covered by HLA class II epitopes. T cell epitopes are less abundant in the shorter N protein: 36% and 11% of the sequence scored as potential HLA class I or 11 epitopes respectively (FIG. 42A). Of the mutations within the alpha or beta variants only a modest percentage affected HLA class I epitopes while within delta and omicron mutations were present in 38% or 48% of such epitopes. HLA class II epitopes within the S protein were less affected and showed mutations rates between 13-27% for the different variants. (FIG. 42B). Most of the epitope mutations within the variants resulted in a reduction or even loss of predicted binding to their corresponding restriction elements. Only one of the mutations within N present in the alpha variant affected an HLA class I epitope; this mutation resulted in increased HLA class I binding to the corresponding peptide (FIG. 42C). HLA class II epitopes of N were not modified by any of the mutations (FIG. 42D).

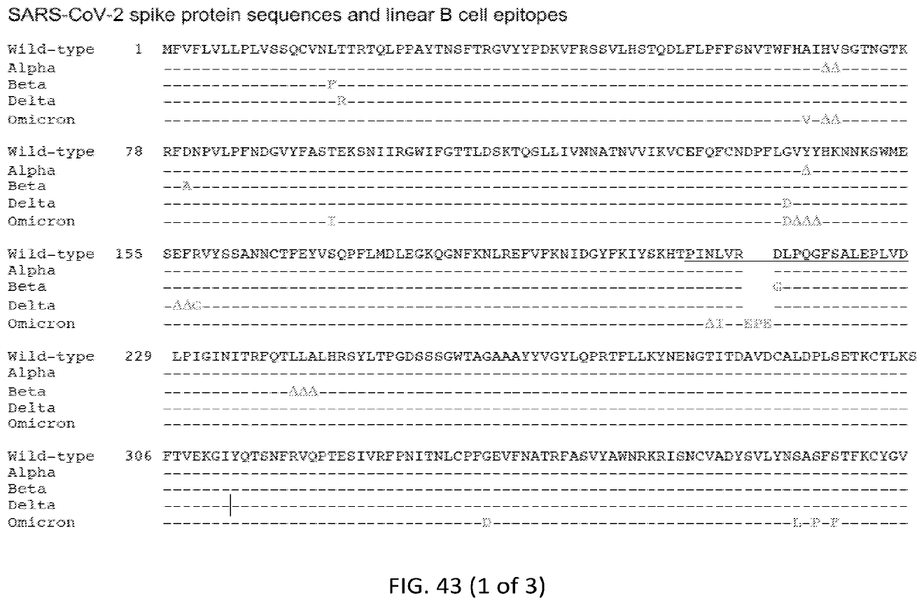

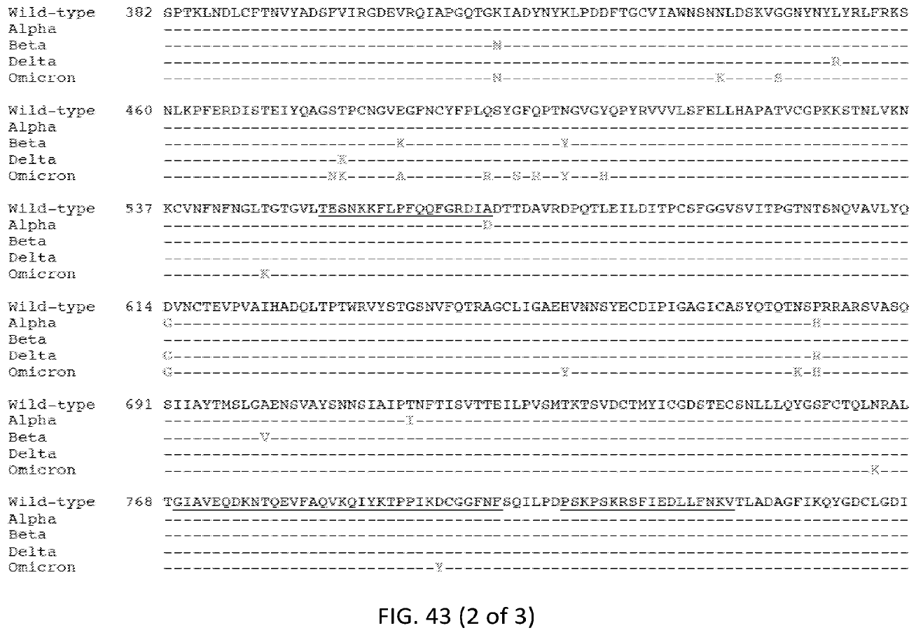

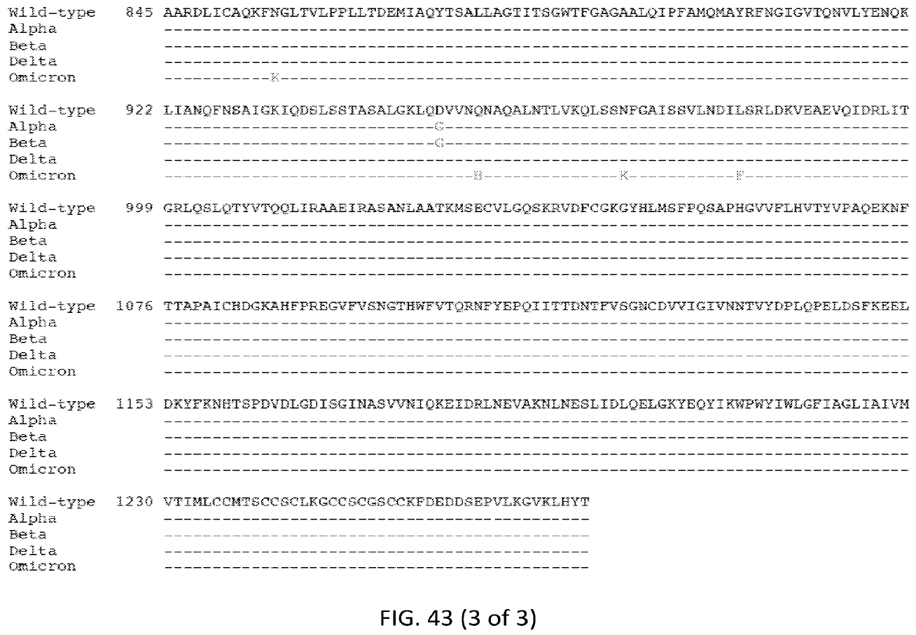

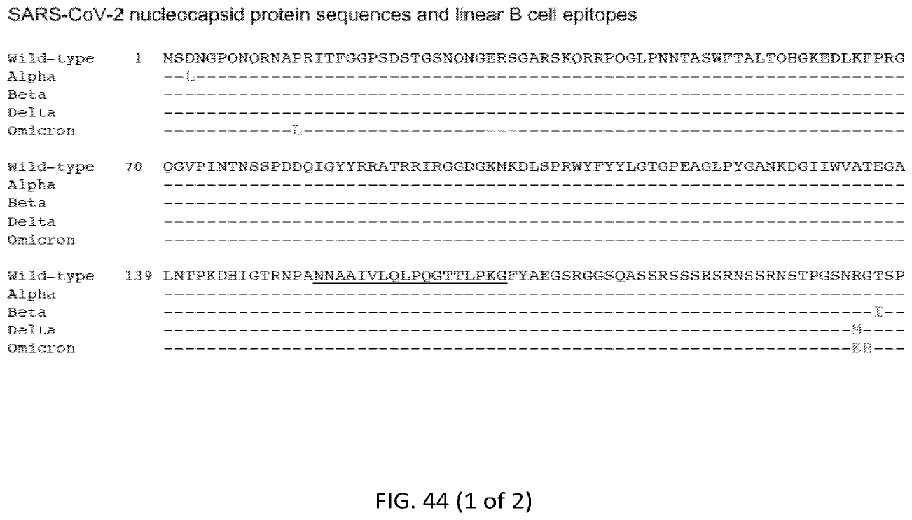

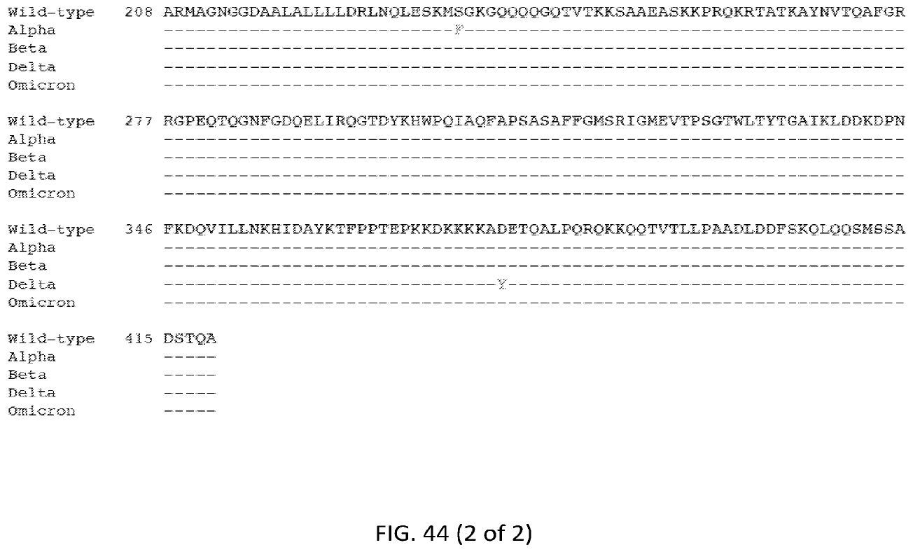

Several linear epitopes have been identified for SARS-CoV-2 S and N proteins (FIG. 43 and FIG. 44, respectively). Of the 4 within the S protein, 3 are mutated in the variants: the 1st epitope that stretches from amino acids 200-220 is mutated in all of the analyzed variants. A 2nd epitope from amino acids 544 to 562 is mutated in the alpha variant and the 3rd epitope from amino acids 569 to 603 is mutated within omicron. The single linear epitope within the N protein is conserved between the variants.

### DISCUSSION

For the hamster challenge study, two AdC vectors that belong to distinct serotypes were used to prevent that Ad capsid-specific antibody responses induced by the prime neutralized the Ad vector used for the boost. Humans only rarely have VNAs to AdC viruses and those who are seropositive have low titers. VNAs to Ad viruses, directed mainly against the hypervariable loops of the hexon protein, can prevent infection of cells with Ad vector vaccines and thereby expression of the transgene product. This in turn reduces the antigenic load and blunts immune responses to the vaccine antigen, which can be problematic for human serotype Ad vectors or for the repeated use of the same Ad vector. The AdC vectors for the S protein carry the full-length unmodified gene from an early SARS-CoV-2 isolate and induce after a boost sustained antibody responses in mice. These data were recapitulated in hamsters which developed binding as well as neutralizing antibodies after vaccination with the AdC-S vectors. A booster effect was seen for binding antibodies for groups 2 and 3 while VNA responses only increased in group 3 mice that received the AdC-S/AdC-gDN vector mixtures. Whether this reflects increased T help due to responses to the N protein or a difference in vector dosing with group 2 hamsters receiving double the dose of the AdC-S vector is unknown. As expected, VNAs cross-reacted, albeit with significantly reduced activity against viral variants of concern.

The N protein is expressed by the AdC vectors as a fusion protein within HSV-gD, which as an inhibitor of the early BTLA-HVEM T cell checkpoint enhances B cell responses and broadens CD8+ T cell responses as was shown with several antigens including the SARS-CoV-2 N protein in mice. Expressing the N protein by an AdC vector within a heterologous viral protein that is expressed on the surface of an infected cells may promote B cell responses to the N protein's linear epitopes but will likely distorted the proteins' structure and destroy conformation-dependent epitopes. Nevertheless, a strong N protein specific antibody response was obtained as demonstrated herein after the boost in hamsters that received the AdC-gDN vaccines.

The N protein as expressed by the AdC vectors induces potent and broad CD8+ T cell responses in mice. Responses are readily detectable but modest in hamsters, which may in part relate to the paucity of hamster-specific antibodies to T cell determinants and key cytokines, which only allowed for testing of circulating CD8+ lymphocytes producing IFN-γ in response to the N peptide pools.

Hamsters were challenged after vaccination to assess if the vaccines protected against disease or infection. It is noteworthy that, mirroring humans, male hamsters were more susceptible to severe disease than female hamsters—one male hamster in the control group died and all male hamsters regardless of vaccination exhibited clinical symptoms throughout the 14-day observation period. Vaccinated female hamsters showed accelerated recovery from symptoms especially after immunization with the AdC-S-AdC-gDN combination vaccine. Both the combination vaccine and the AdC-S vaccine provided solid protection against weight loss. Despite reducing symptoms, none of the vaccine regimens prevented infection; in all animals viral RNA and sgRNA were recovered at high titers from their oral cavities with only minor reductions in groups 2 and 3 on day 2 after challenge. In the same token only one group 2 animal was protected against a pulmonary infection while all other animals had detectable titers of viral RNA and sgRNA on day 4 after challenge which declined by day 14. Lung virus RNA titers on day 4 were significantly lower in groups 2 and 3 and by day 14, 3 out of 4 group 3 and 1 out of 4 group 2 animals scored negative for lung sgRNA titers while control animals and animals vaccinated only with the AdC-gDN vaccine retained titers of approximately 10⁵ copies per ml. Not only did vaccinated animals become infected, but they also transmitted the virus to unvaccinated hamsters that were placed into their cages as of day 4 after challenge (data not shown). Lung pathology mirrored lung sgRNA titers and again females had less lung disease compared to males and group 3 animals tended to recover faster than group 2 animals while group 1 animals showed no difference compared to control animals, although addition of the AdC-gDN vaccine reduced disease and accelerated recovery in animals that were also vaccinated with the AdC-S vaccine. By itself, the AdC-gDN vaccine only induced marginal protection, which contrasts data from another group which reported T cell-mediated protection against COVID-19 disease and a reduction in viral loads upon intravenous immunization of hamsters with a human serotype 5 Ad vector expressing the N protein. Differences in results may reflect the distinct immunization protocols or differences in the challenge.

In the present study, the contribution of the AdC-gDN vaccine to protection induced by the AdC-S vaccines was not solely mediated by T cells, which have the advantage that upon antigen-driven activation they persist for very long periods of time, which would likely extend duration of protection well beyond the 4-6 months that current vaccines offer. Antibodies to the N protein strong positive correlations with relative weight of the animals and inverse correlations with disease parameters such as viral loads and lung pathology in the late euthanasia group indicating that the N protein-specific antibodies contribute to accelerated recovery from the infection.

### Enumerated Embodiments

The following enumerated embodiments are provided, the numbering of which is not to be construed as designating levels of importance.

Embodiment 1 provides an immunogenic composition comprising an effective amount of a first adenoviral vector comprising a first nucleotide sequence encoding a first SARS-CoV-2 protein and a second adenoviral vector comprising a second nucleotide sequence encoding a second SARS-CoV-2 protein.

Embodiment 2 provides the immunogenic composition of embodiment 1, wherein the first SARS-CoV-2 protein is SARS-CoV-2 spike (S) protein and the second SARS-CoV-2 protein is SARS-CoV-2 nucleocapsid (N) protein.

Embodiment 3 provides the immunogenic composition of embodiment 2, wherein the nucleocapsid protein is fused to a domain of herpes simplex virus glycoprotein D, thereby forming a fusion protein.

Embodiment 4 provides the immunogenic composition of embodiment 2 or embodiment 3, wherein the SARS-CoV-2 spike protein comprises SEQ ID NO: 2 or SEQ ID NO: 6, and the SARS-CoV-2 nucleocapsid protein comprises SEQ ID NO: 4.

Embodiment 5 provides the immunogenic composition of any one of embodiments 1-4, wherein the first nucleotide sequence comprises SEQ ID NO: 1 or SEQ ID NO: 5, and the second nucleotide sequence comprises SEQ ID NO: 3.

Embodiment 6 provides the immunogenic composition of any one of embodiments 1-5, wherein the first adenoviral vector and the second adenoviral vector are replication-defective.

Embodiment 7 provides the immunogenic composition of any one of embodiments 1-6, wherein the first adenoviral vector and the second adenoviral vector are of chimpanzee origin.

Embodiment 8 provides the immunogenic composition of any one of embodiments 1-7, wherein the first adenoviral vector is serotype-6 (AdC6) or serotype-7 (AdC7).

Embodiment 9 provides the immunogenic composition of any one of embodiments 1-8, wherein the second adenoviral vector is serotype-6 (AdC6) or serotype-7 (AdC7).

Embodiment 10 provides the immunogenic composition of any one of embodiments 1-9, wherein the first adenoviral vector and the second adenoviral vector are of the same serotype.

Embodiment 11 provides the immunogenic composition of any one of embodiments 1-9, wherein the first adenoviral vector and the second adenoviral vector are of different serotypes.

Embodiment 12 provides the immunogenic composition of any one of embodiments 1-11, wherein the immunogenic composition further comprises a pharmaceutically acceptable vehicle.

Embodiment 13 provides a viral vector comprising a first nucleotide sequence encoding a first SARS-CoV-2 protein and a second nucleotide sequence encoding a second SARS-CoV-2 protein.

Embodiment 14 provides the viral vector of embodiment 13, wherein the first SARS-CoV-2 protein is SARS-CoV-2 spike (S) protein and the second SARS-CoV-2 protein is SARS-CoV-2 nucleocapsid (N) protein.

Embodiment 15 provides the viral vector of embodiment 14, wherein the nucleocapsid protein is fused to a domain of herpes simplex virus glycoprotein D, thereby forming a fusion protein.

Embodiment 16 provides the viral vector of embodiment 14 or embodiment 15, wherein the SARS-CoV-2 spike protein comprises SEQ ID NO: 2 or SEQ ID NO: 6, and the SARS-CoV-2 nucleocapsid protein comprises SEQ ID NO: 4.

Embodiment 17 provides the viral vector of any one of embodiments 13-16, wherein the first nucleotide sequence comprises SEQ ID NO: 1 or SEQ ID NO: 5, and the second nucleotide sequence comprises SEQ ID NO: 3.

Embodiment 18 provides the viral vector of any one of embodiments 13-17, wherein the viral vector is an adenoviral vector.

Embodiment 19 provides the viral vector of embodiment 18, wherein the adenoviral vector is replication-defective.

Embodiment 20 provides the viral vector of embodiment 18 or embodiment 19, wherein the adenoviral vector is of chimpanzee origin.

Embodiment 21 provides the viral vector of any one of embodiments 18-20, wherein the adenoviral vector is serotype-6 (AdC6) or serotype-7 (AdC7).

Embodiment 22 provides a method of stimulating an immune response in a subject, comprising administering to the subject an effective amount of the immunogenic composition of any one of embodiments 1-12 or an effective amount of the viral vector of any one of claims 13-21.

Embodiment 23 provides the method of embodiment 22, wherein the immunogenic composition or the viral vector is delivered by a route selected from the group consisting of oral route, inhalation route, nasal route, nebulization route, intravenous injection, intraperitoneal injection, intramuscular injection, subcutaneous injection, transdermal injection, and any combination thereof.

Embodiment 24 provides a method for treating or preventing a coronavirus infection in a subject, comprising administering to the subject an effective amount of the immunogenic composition of any one of embodiments 1-12 or an effective amount of the viral vector of any one of claims 13-21.

Embodiment 25 provides the method of embodiment 24, wherein the immunogenic composition or the viral vector is delivered by a route selected from the group consisting of oral route, inhalation route, nasal route, nebulization route, intravenous injection, intraperitoneal injection, intramuscular injection, subcutaneous injection, transdermal injection, and any combination thereof.

Embodiment 26 provides the method of embodiment 24, wherein the coronavirus infection is caused by the SARS-CoV-2 virus.

Embodiment 27 provides a method of stimulating an immune response in a subject, the method comprising administering to the subject: (i) an effective amount of a first adenoviral vector comprising a first nucleotide sequence encoding a first SARS-CoV-2 protein; and (ii) a second adenoviral vector comprising a second nucleotide sequence encoding a second SARS-CoV-2 protein.

Embodiment 28 provides a method for treating or preventing a coronavirus infection in a subject, the method comprising administering to the subject: (i) an effective amount of a first adenoviral vector comprising a first nucleotide sequence encoding a first SARS-CoV-2 protein; and (ii) a second adenoviral vector comprising a second nucleotide sequence encoding a second SARS-CoV-2 protein.

Embodiment 29 provides the method of embodiment 27 or embodiment 28, wherein the first SARS-CoV-2 protein is SARS-CoV-2 spike (S) protein and the second SARS-CoV-2 protein is SARS-CoV-2 nucleocapsid (N) protein.

Embodiment 30 provides the method of embodiment 29, wherein the nucleocapsid protein is fused to a domain of herpes simplex virus glycoprotein D, thereby forming a fusion protein.

Embodiment 31 provides the method of embodiment 29 or embodiment 30, wherein the SARS-CoV-2 spike protein comprises SEQ ID NO: 2 or SEQ ID NO: 6, and the SARS-CoV-2 nucleocapsid protein comprises SEQ ID NO: 4.

Embodiment 32 provides the method of any one of embodiments 27-31, wherein the first nucleotide sequence comprises SEQ ID NO: 1 or SEQ ID NO: 5, and the second nucleotide sequence comprises SEQ ID NO: 3.

Embodiment 33 provides the method of any one of embodiments 27-32, wherein the first adenoviral vector and the second adenoviral vector are replication-defective.

Embodiment 34 provides the method of any one of embodiments 27-33, wherein the first adenoviral vector is serotype-6 (AdC6) or serotype-7 (AdC7).

Embodiment 35 provides the method of any one of embodiments 27-34, wherein the second adenoviral vector is serotype-6 (AdC6) or serotype-7 (AdC7).

Embodiment 36 provides the method of any one of embodiments 27-35, wherein the first adenoviral vector and the second adenoviral vector are of the same serotype.

Embodiment 37 provides the method of any one of embodiments 27-35, wherein the first adenoviral vector and the second adenoviral vector are of different serotypes.

Embodiment 38 provides the method of any one of embodiments 27-37, wherein administration of the first adenoviral vector and administration of the second adenoviral vector each independently comprises a route selected from the group consisting of oral route, inhalation route, nasal route, nebulization route, intravenous injection, intraperitoneal injection, intramuscular injection, subcutaneous injection, transdermal injection, and any combination thereof.

Embodiment 39 provides the method of any one of embodiments 27-38, wherein the first adenoviral vector is administered prior to the second adenoviral vector.

Embodiment 40 provides the method of any one of embodiments 27-38, wherein the second adenoviral vector is administered prior to the first adenoviral vector.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An immunogenic composition comprising an effective amount of a first adenoviral vector comprising a first nucleotide sequence encoding a first SARS-CoV-2 protein and a second adenoviral vector comprising a second nucleotide sequence encoding a second SARS-CoV-2 protein.

2. The immunogenic composition of claim 1, wherein the first SARS-CoV-2 protein is SARS-CoV-2 spike (S) protein and the second SARS-CoV-2 protein is SARS-CoV-2 nucleocapsid (N) protein.

3. The immunogenic composition of claim 2, wherein the nucleocapsid protein is fused to a domain of herpes simplex virus glycoprotein D, thereby forming a fusion protein.

4. The immunogenic composition of claim 2, wherein the SARS-CoV-2 spike protein comprises SEQ ID NO: 2 or SEQ ID NO: 6, and the SARS-CoV-2 nucleocapsid protein comprises SEQ ID NO: 4.

5. The immunogenic composition of claim 1, wherein the first nucleotide sequence comprises SEQ ID NO: 1 or SEQ ID NO: 5, and the second nucleotide sequence comprises SEQ ID NO: 3.

6. The immunogenic composition of claim 1, wherein the first adenoviral vector and the second adenoviral vector are replication-defective.

7. The immunogenic composition of claim 1, wherein the first adenoviral vector and the second adenoviral vector are of chimpanzee origin.

8. The immunogenic composition of claim 1, wherein the first adenoviral vector is serotype-6 (AdC6) or serotype-7 (AdC7).

9. The immunogenic composition of claim 1, wherein the second adenoviral vector is serotype-6 (AdC6) or serotype-7 (AdC7).

10. The immunogenic composition of claim 1, wherein the first adenoviral vector and the second adenoviral vector are of the same serotype.

11. The immunogenic composition of claim 1, wherein the first adenoviral vector and the second adenoviral vector are of different serotypes.

12. The immunogenic composition of claim 1, wherein the immunogenic composition further comprises a pharmaceutically acceptable vehicle.

13. A viral vector comprising a first nucleotide sequence encoding a first SARS-CoV-2 protein and a second nucleotide sequence encoding a second SARS-CoV-2 protein.

14. The viral vector of claim 13, wherein the first SARS-CoV-2 protein is SARS-CoV-2 spike (S) protein and the second SARS-CoV-2 protein is SARS-CoV-2 nucleocapsid (N) protein.

15. The viral vector of claim 14, wherein the nucleocapsid protein is fused to a domain of herpes simplex virus glycoprotein D, thereby forming a fusion protein.

16. The viral vector of claim 14, wherein the SARS-CoV-2 spike protein comprises SEQ ID NO: 2 or SEQ ID NO: 6, and the SARS-CoV-2 nucleocapsid protein comprises SEQ ID NO: 4.

17. The viral vector of claim 13, wherein the first nucleotide sequence comprises SEQ ID NO: 1 or SEQ ID NO: 5, and the second nucleotide sequence comprises SEQ ID NO: 3.

18. The viral vector of claim 13, wherein the viral vector is an adenoviral vector.

19. The viral vector of claim 18, wherein the adenoviral vector is replication-defective.

20. The viral vector of claim 18, wherein the adenoviral vector is of chimpanzee origin.

21. The viral vector of claim 18, wherein the adenoviral vector is serotype-6 (AdC6) or serotype-7 (AdC7).

22. A method of stimulating an immune response in a subject, comprising administering to the subject an effective amount of the immunogenic composition of claim 1.

23. The method of claim 22, wherein the immunogenic composition is delivered by a route selected from the group consisting of oral route, inhalation route, nasal route, nebulization route, intravenous injection, intraperitoneal injection, intramuscular injection, subcutaneous injection, transdermal injection, and any combination thereof.

24. A method for treating or preventing a coronavirus infection in a subject, comprising administering to the subject an effective amount of the immunogenic composition of claim 1.

25. The method of claim 24, wherein the immunogenic composition is delivered by a route selected from the group consisting of oral route, inhalation route, nasal route, nebulization route, intravenous injection, intraperitoneal injection, intramuscular injection, subcutaneous injection, transdermal injection, and any combination thereof.

26. The method of claim 24, wherein the coronavirus infection is caused by the SARS-CoV-2 virus.

27. A method of stimulating an immune response in a subject, the method comprising administering to the subject: (i) an effective amount of a first adenoviral vector comprising a first nucleotide sequence encoding a first SARS-CoV-2 protein; and (ii) a second adenoviral vector comprising a second nucleotide sequence encoding a second SARS-CoV-2 protein.

28. A method for treating or preventing a coronavirus infection in a subject, the method comprising administering to the subject: (i) an effective amount of a first adenoviral vector comprising a first nucleotide sequence encoding a first SARS-CoV-2 protein; and (ii) a second adenoviral vector comprising a second nucleotide sequence encoding a second SARS-CoV-2 protein.

29. The method of claim 27, wherein the first SARS-CoV-2 protein is SARS-CoV-2 spike (S) protein and the second SARS-CoV-2 protein is SARS-CoV-2 nucleocapsid (N) protein.

30. The method of claim 29, wherein the nucleocapsid protein is fused to a domain of herpes simplex virus glycoprotein D, thereby forming a fusion protein.

31. The method of claim 29, wherein the SARS-CoV-2 spike protein comprises SEQ ID NO: 2 or SEQ ID NO: 6, and the SARS-CoV-2 nucleocapsid protein comprises SEQ ID NO: 4.

32. The method of claim 27, wherein the first nucleotide sequence comprises SEQ ID NO: 1 or SEQ ID NO: 5, and the second nucleotide sequence comprises SEQ ID NO: 3.

33. The method of claim 27, wherein the first adenoviral vector and the second adenoviral vector are replication-defective.

34. The method of claim 27, wherein the first adenoviral vector is serotype-6 (AdC6) or serotype-7 (AdC7).

35. The method of claim 27, wherein the second adenoviral vector is serotype-6 (AdC6) or serotype-7 (AdC7).

36. The method of claim 27, wherein the first adenoviral vector and the second adenoviral vector are of the same serotype.

37. The method of claim 27, wherein the first adenoviral vector and the second adenoviral vector are of different serotypes.

38. The method of claim 27, wherein administration of the first adenoviral vector and administration of the second adenoviral vector each independently comprises a route selected from the group consisting of oral route, inhalation route, nasal route, nebulization route, intravenous injection, intraperitoneal injection, intramuscular injection, subcutaneous injection, transdermal injection, and any combination thereof.

39. The method of claim 27, wherein the first adenoviral vector is administered prior to the second adenoviral vector.

40. The method of claim 27, wherein the second adenoviral vector is administered prior to the first adenoviral vector.


SEQUENCE LISTING (160 sequences)

SEQ ID NO: 1 - DNA, 3816 bp, Artificial Sequence;SARS-CoV-2 Spike
  Unk
tttgtgtttctggtgcttcttccattggttagcagtcaatgcgttaatcttaccacccgcactcagctgcctccagcttatactaatagtttcactcgcggtgtgtactatccagacaaagttttccgcagctccgtcctccattcaactcaagacctgttccttccatttttcagtaatgtcacttggtttcatgctattcacgtttctggtaccaatggcactaaacgcttcgataaccctgttttgcctttcaatgacggtgtgtatttcgcctcaacagaaaaatcaaacatcatccgcggttggatttttggcactaccctcgatagtaagacccagtccctgctgatcgtcaataacgccacaaatgtggttatcaaagtttgtgagttccaattttgcaacgacccatttcttggtgtttactaccataagaacaacaaatcatggatggagtctgagttccgcgtctatagcagcgccaacaactgtactttcgagtatgtctcccagcctttccttatggatcttgagggcaaacaggggaatttcaaaaaccttcgcgagtttgtgtttaagaacatcgacggttattttaaaatttacagcaaacatactcctatcaacttggttcgcgacctcccacaggggttctccgccctggaacctctggttgatttgcctatcggcatcaacattactcgcttccagaccttgctcgcccttcaccgctcatacctgacccctggggatagttcatctggctggactgctggtgccgccgcatactacgtgggctatttgcaacctcgcacttttctccttaagtataacgaaaacggtactattaccgacgccgttgattgtgcactcgatcctttgagcgaaaccaagtgcactttgaaaagttttaccgtggaaaaagggatttaccaaacatctaattttcgcgtgcagccaaccgaatccatcgtccgcttcccaaatatcactaacttgtgtcctttcggggaggttttcaatgctacacgctttgcatctgtgtatgcttggaaccgcaagcgcatttcaaactgcgttgcagactactcagttttgtacaacagcgcttcattcagcacatttaagtgctatggtgtgagcccaaccaagctcaacgatctttgctttaccaatgtgtacgctgactcttttgtcatccgcggtgatgaggtccgccagattgctccagggcagacaggtaaaattgccgattacaattacaaactgcctgatgattttaccgggtgcgttattgcctggaacagcaataatttggatagcaaagtcgggggtaactacaactatctttaccgcttgtttcgcaagtctaatctgaagccattcgaacgcgatatcagcactgagatctatcaggccgggtcaaccccatgtaacggggttgaagggttcaactgctatttccctctccaatcttacggtttccagccaaccaatggggtcggctaccagccatatcgcgtcgtcgtcctcagctttgagctgcttcacgctcctgcaaccgtctgtggccctaagaagtcaacaaacctcgtgaaaaacaaatgtgtcaacttcaacttcaatggcctcaccggcacaggcgttctgactgaaagcaataaaaagttcttgccatttcagcaattcggtcgcgatattgctgatacaacagatgccgttcgcgacccacagaccctggaaattctcgatattactccttgttcttttggcggggtgagcgtgattactccagggacaaatacaagtaaccaagttgcagtgttgtatcaggacgtcaactgtaccgaggttcctgtcgcaatccacgccgatcaacttacacctacatggcgcgtgtatagcactgggagtaatgtcttccaaacacgcgctgggtgccttattggggccgaacatgttaataactcatatgaatgcgacatcccaatcggcgccgggatctgcgctagctaccagactcagacaaacagtcctcgccgcgctcgctccgtggcaagccagtccattatcgcctatactatgtctctcggtgctgaaaactccgtcgcatatagtaataatagcatcgccattccaaccaattttacaattagcgttacaaccgagattctgcctgtctccatgaccaaaacttccgtcgactgcaccatgtatatctgtggcgacagcaccgagtgctccaatctcttgcttcaatacgggagcttctgcactcaactgaaccgcgccttgactggtattgcagttgaacaagataaaaatacccaggaggtctttgcacaagtgaagcaaatttacaaaacccctcctatcaaggactgtgggggctttaatttctcacaaatcctgcctgatccatccaaacctagcaaacgctcattcattgaggacctgctctttaataaagttacattggctgacgctggttttattaaacaatatggtgactgtctcggtgacatcgctgcccgcgacttgatttgtgcacaaaagttcaatgggctcactgttctgccacctttgctcaccgatgagatgatcgcacagtatacaagtgccttgctggccggcaccatcacctccggttggacttttggtgccggtgcagctttgcaaatcccttttgcaatgcaaatggcttatcgctttaacgggatcggggtgactcagaatgttctgtatgagaaccaaaaactgatcgccaatcaatttaactcagccatcggcaagattcaagattcactgtcatctacagcctcagcattgggtaagttgcaggacgtcgtcaaccaaaacgctcaggcactgaacacattggtcaaacaacttagctctaatttcggtgccatcagtagtgtgttgaatgacattcttagccgcctggacaaagtggaagccgaggttcagatcgaccgccttattactggtcgcttgcagtctctgcagacatatgttacccaacaactcattcgcgccgctgaaattcgcgccagcgccaacctggctgccacaaagatgtccgaatgcgtcttggggcaaagcaagcgcgttgatttctgcggcaaaggttatcacttgatgtcttttcctcaatctgcacctcacggggtggtgttcttgcacgttacttacgttccagcacaggagaaaaacttcacaacagcacctgctatttgtcatgacggtaaggctcacttccctcgcgagggtgtctttgtcagtaacggtactcactggtttgtgacccagcgcaacttttatgagcctcaaatcatcacaaccgacaacacctttgtttccgggaactgtgatgttgtcattggtatcgtcaacaacacagtctatgaccctcttcaaccagaactggactcatttaaggaagagctcgacaagtattttaaaaatcacacctctcctgatgtggatcttggggatatcagtggtattaacgctagtgtggttaatatccaaaaagaaatcgatcgcctgaatgaagtcgctaaaaatctgaatgaaagtttgatcgacttgcaagaacttgggaagtacgaacaatatattaaatggccttggtacatttggctcggctttatcgctggcctcattgccatcgttatggttacaattatgctctgctgcatgacatcatgctgcagttgcttgaaaggttgttgtagttgtgggtcttgctgtaagtttgatgaggatgactctgagcctgttcttaagggtgtgaaattgcactacacc

SEQ ID NO: 2 - protein, 1273 aa, Artificial Sequence;SARS-CoV-2 Spike
  Unk
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDCGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - DNA, 1254 bp, Artificial Sequence;SARS-CoV-2 Nucleocapsid
  Unk
tccgataacggcccacaaaatcaacgcaacgcacctcgcattacctttggcggtccttccgattctaccggttcaaaccaaaatggggagcgcagtggtgcccgctcaaaacaacgccgccctcaaggtcttcctaataatactgcttcatggttcaccgctttgacacaacatggtaaagaggatctgaaatttccacgcgggcaaggggtcccaattaacaccaattcctctccagatgaccagattgggtactatcgccgcgctactcgccgcatccgcggtggggacggtaaaatgaaagatctgtcccctcgctggtatttttattatcttgggactgggcctgaagcaggtttgccatacggggccaataaagacggtattatctgggttgcaacagaaggcgccctgaatactccaaaggatcatattgggactcgcaatccagcaaataatgcagctattgttttgcagctccctcagggtaccacccttcctaagggtttctatgcagagggttctcgcggtggttctcaggcatcctcccgctcatcttctcgctcacgcaacagttctcgcaattcaacaccaggttcaaatcgcggtaccagcccagcacgcatggctggtaatggtggtgacgctgctttggcattgttgctgctcgatcgcctgaaccagctggaatccaagatgagtgggaaaggtcagcaacaacagggtcagacagtcactaaaaagtctgccgctgaggcttctaaaaagccacgccagaagcgcacagcaactaaggcttacaatgtgacccaggcttttggtcgccgcggtccagaacagacacaaggtaactttggtgatcaggagctcattcgccaaggcaccgattataaacattggccacagattgcccaattcgcaccatcagcttcagccttctttggcatgtcacgcatcggcatggaggtgactccttccgggacatggttgacatacactggtgcaatcaaactggacgataaggaccctaattttaaagatcaggtcatcctgcttaacaaacatattgatgcctacaagacttttccacctaccgaacctaagaaagacaagaaaaagaaggcagatgagactcaggctttgcctcaacgccaaaagaagcaacagacagttacactccttccagcagccgatcttgatgacttcagtaaacaactgcaacaatctatgagctctgcagatagtactcaggcc

SEQ ID NO: 4 - protein, 419 aa, Artificial Sequence;SARS-CoV-2 Nucleocapsid
  Unk
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSNRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 5 - DNA, 3822 bp, Artificial Sequence;Spike Age I mutant
  Unk
atgtttgtgtttctggtgcttcttccattggttagcagtcaatgcgttaatcttaccacccgcactcagctgcctccagcttatactaatagtttcactcgcggtgtgtactatccagacaaagttttccgcagctccgtcctccattcaactcaagacctgttccttccatttttcagtaatgtcacttggtttcatgctattcacgtttctggtaccaatggcactaaacgcttcgataaccctgttttgcctttcaatgacggtgtgtatttcgcctcaacagaaaaatcaaacatcatccgcggttggatttttggcactaccctcgatagtaagacccagtccctgctgatcgtcaataacgccacaaatgtggttatcaaagtttgtgagttccaattttgcaacgacccatttcttggtgtttactaccataagaacaacaaatcatggatggagtctgagttccgcgtctatagcagcgccaacaactgtactttcgagtatgtctcccagcctttccttatggatcttgagggcaaacaggggaatttcaaaaaccttcgcgagtttgtgtttaagaacatcgacggttattttaaaatttacagcaaacatactcctatcaacttggttcgcgacctcccacaggggttctccgccctggaacctctggttgatttgcctatcggcatcaacattactcgcttccagaccttgctcgcccttcaccgctcatacctgacccctggggatagttcatctggctggactgctggtgccgccgcatactacgtgggctatttgcaacctcgcacttttctccttaagtataacgaaaacggtactattaccgacgccgttgattgtgcactcgatcctttgagcgaaaccaagtgcactttgaaaagttttaccgtggaaaaagggatttaccaaacatctaattttcgcgtgcagccaaccgaatccatcgtccgcttcccaaatatcactaacttgtgtcctttcggggaggttttcaatgctacacgctttgcatctgtgtatgcttggaaccgcaagcgcatttcaaactgcgttgcagactactcagttttgtacaacagcgcttcattcagcacatttaagtgctatggtgtgagcccaaccaagctcaacgatctttgctttaccaatgtgtacgctgactcttttgtcatccgcggtgatgaggtccgccagattgctccagggcagacaggtaaaattgccgattacaattacaaactgcctgatgattttaccgggtgcgttattgcctggaacagcaataatttggatagcaaagtcgggggtaactacaactatctttaccgcttgtttcgcaagtctaatctgaagccattcgaacgcgatatcagcactgagatctatcaggccgggtcaaccccatgtaacggggttgaagggttcaactgctatttccctctccaatcttacggtttccagccaaccaatggggtcggctaccagccatatcgcgtcgtcgtcctcagctttgagctgcttcacgctcctgcaaccgtctgtggccctaagaagtcaacaaacctcgtgaaaaacaaatgtgtcaacttcaacttcaatggcctcaccggtacaggcgttctgactgaaagcaataaaaagttcttgccatttcagcaattcggtcgcgatattgctgatacaacagatgccgttcgcgacccacagaccctggaaattctcgatattactccttgttcttttggcggggtgagcgtgattactccagggacaaatacaagtaaccaagttgcagtgttgtatcaggacgtcaactgtaccgaggttcctgtcgcaatccacgccgatcaacttacacctacatggcgcgtgtatagcactgggagtaatgtcttccaaacacgcgctgggtgccttattggggccgaacatgttaataactcatatgaatgcgacatcccaatcggcgccgggatctgcgctagctaccagactcagacaaacagtcctcgccgcgctcgctccgtggcaagccagtccattatcgcctatactatgtctctcggtgctgaaaactccgtcgcatatagtaataatagcatcgccattccaaccaattttacaattagcgttacaaccgagattctgcctgtctccatgaccaaaacttccgtcgactgcaccatgtatatctgtggcgacagcaccgagtgctccaatctcttgcttcaatacgggagcttctgcactcaactgaaccgcgccttgactggtattgcagttgaacaagataaaaatacccaggaggtctttgcacaagtgaagcaaatttacaaaacccctcctatcaaggactgtgggggctttaatttctcacaaatcctgcctgatccatccaaacctagcaaacgctcattcattgaggacctgctctttaataaagttacattggctgacgctggttttattaaacaatatggtgactgtctcggtgacatcgctgcccgcgacttgatttgtgcacaaaagttcaatgggctcactgttctgccacctttgctcaccgatgagatgatcgcacagtatacaagtgccttgctggccggcaccatcacctccggttggacttttggtgccggtgcagctttgcaaatcccttttgcaatgcaaatggcttatcgctttaacgggatcggggtgactcagaatgttctgtatgagaaccaaaaactgatcgccaatcaatttaactcagccatcggcaagattcaagattcactgtcatctacagcctcagcattgggtaagttgcaggacgtcgtcaaccaaaacgctcaggcactgaacacattggtcaaacaacttagctctaatttcggtgccatcagtagtgtgttgaatgacattcttagccgcctggacaaagtggaagccgaggttcagatcgaccgccttattactggtcgcttgcagtctctgcagacatatgttacccaacaactcattcgcgccgctgaaattcgcgccagcgccaacctggctgccacaaagatgtccgaatgcgtcttggggcaaagcaagcgcgttgatttctgcggcaaaggttatcacttgatgtcttttcctcaatctgcacctcacggggtggtgttcttgcacgttacttacgttccagcacaggagaaaaacttcacaacagcacctgctatttgtcatgacggtaaggctcacttccctcgcgagggtgtctttgtcagtaacggtactcactggtttgtgacccagcgcaacttttatgagcctcaaatcatcacaaccgacaacacctttgtttccgggaactgtgatgttgtcattggtatcgtcaacaacacagtctatgaccctcttcaaccagaactggactcatttaaggaagagctcgacaagtattttaaaaatcacacctctcctgatgtggatcttggggatatcagtggtattaacgctagtgtggttaatatccaaaaagaaatcgatcgcctgaatgaagtcgctaaaaatctgaatgaaagtttgatcgacttgcaagaacttgggaagtacgaacaatatattaaatggccttggtacatttggctcggctttatcgctggcctcattgccatcgttatggttacaattatgctctgctgcatgacatcatgctgcagttgcttgaaaggttgttgtagttgtgggtcttgctgtaagtttgatgaggatgactctgagcctgttcttaagggtgtgaaattgcactacacctaa

SEQ ID NO: 6 - protein, 1273 aa, Artificial Sequence;Spike Age I Mutant; identical to SEQ ID NO: 2
  Unk

SEQ ID NO: 7 - protein, 15 aa, Artificial Sequence;Peptide 1
  Unk
MSDNGPQNQRNAPRI

SEQ ID NO: 8 - protein, 15 aa, Artificial Sequence;Peptide 2
  Unk
PQNQRNAPRITFGGP

SEQ ID NO: 9 - protein, 15 aa, Artificial Sequence;Peptide 3
  Unk
NAPRITFGGPSDSTG

SEQ ID NO: 10 - protein, 15 aa, Artificial Sequence;Peptide 4
  Unk
TFGGPSDSTGSNQNG

SEQ ID NO: 11 - protein, 15 aa, Artificial Sequence;Peptide 5
  Unk
SDSTGSNQNGERSGA

SEQ ID NO: 12 - protein, 15 aa, Artificial Sequence;Peptide 6
  Unk
SNQNGERSGARSKQR

SEQ ID NO: 13 - protein, 15 aa, Artificial Sequence;Peptide 7
  Unk
ERSGARSKQRRPQGL

SEQ ID NO: 14 - protein, 15 aa, Artificial Sequence;Peptide 8
  Unk
RSKQRRPQGLPNNTA

SEQ ID NO: 15 - protein, 15 aa, Artificial Sequence;Peptide 9
  Unk
RPQGLPNNTASWFTA

SEQ ID NO: 16 - protein, 15 aa, Artificial Sequence;Peptide 10
  Unk
PNNTASWFTALTQHG

SEQ ID NO: 17 - protein, 15 aa, Artificial Sequence;Peptide 11
  Unk
SWFTALTQHGKEDLK

SEQ ID NO: 18 - protein, 15 aa, Artificial Sequence;Peptide 12
  Unk
LTQHGKEDLKFPRGQ

SEQ ID NO: 19 - protein, 15 aa, Artificial Sequence;Peptide 13
  Unk
KEDLKFPRGQGVPIN

SEQ ID NO: 20 - protein, 15 aa, Artificial Sequence;Peptide 14
  Unk
FPRGQGVPINTNSSP

SEQ ID NO: 21 - protein, 15 aa, Artificial Sequence;Peptide 15
  Unk
GVPINTNSSPDDQIG

SEQ ID NO: 22 - protein, 15 aa, Artificial Sequence;Peptide 16
  Unk
TNSSPDDQIGYYRRA

SEQ ID NO: 23 - protein, 15 aa, Artificial Sequence;Peptide 17
  Unk
DDQIGYYRRATRRIR

SEQ ID NO: 24 - protein, 15 aa, Artificial Sequence;Peptide 18
  Unk
YYRRATRRIRGGDGK

SEQ ID NO: 25 - protein, 15 aa, Artificial Sequence;Peptide 19
  Unk
TRRIRGGDGKMKDLS

SEQ ID NO: 26 - protein, 15 aa, Artificial Sequence;Peptide 20
  Unk
GGDGKMKDLSPRWYF

SEQ ID NO: 27 - protein, 15 aa, Artificial Sequence;Peptide 21
  Unk
MKDLSPRWYFYYLGT

SEQ ID NO: 28 - protein, 15 aa, Artificial Sequence;Peptide 22
  Unk
PRWYFYYLGTGPEAG

SEQ ID NO: 29 - protein, 15 aa, Artificial Sequence;Peptide 23
  Unk
YYLGTGPEAGLPYGA

SEQ ID NO: 30 - protein, 15 aa, Artificial Sequence;Peptide 24
  Unk
GPEAGLPYGANKDGI

SEQ ID NO: 31 - protein, 15 aa, Artificial Sequence;Peptide 25
  Unk
LPYGANKDGIIWVAT

SEQ ID NO: 32 - protein, 15 aa, Artificial Sequence;Peptide 26
  Unk
NKDGIIWVATEGALN

SEQ ID NO: 33 - protein, 15 aa, Artificial Sequence;Peptide 27
  Unk
IWVATEGALNTPKDH

SEQ ID NO: 34 - protein, 15 aa, Artificial Sequence;Peptide 28
  Unk
EGALNTPKDHIGTRN

SEQ ID NO: 35 - protein, 15 aa, Artificial Sequence;Peptide 29
  Unk
TPKDHIGTRNPANNA

SEQ ID NO: 36 - protein, 15 aa, Artificial Sequence;Peptide 30
  Unk
IGTRNPANNAAIVLQ

SEQ ID NO: 37 - protein, 15 aa, Artificial Sequence;Peptide 31
  Unk
PANNAAIVLQLPQGT

SEQ ID NO: 38 - protein, 15 aa, Artificial Sequence;Peptide 32
  Unk
AIVLQLPQGTTLPKG

SEQ ID NO: 39 - protein, 15 aa, Artificial Sequence;Peptide 33
  Unk
LPQGTTLPKGFYAEG

SEQ ID NO: 40 - protein, 15 aa, Artificial Sequence;Peptide 34
  Unk
TLPKGFYAEGSRGGS

SEQ ID NO: 41 - protein, 15 aa, Artificial Sequence;Peptide 35
  Unk
FYAEGSRGGSQASSR

SEQ ID NO: 42 - protein, 15 aa, Artificial Sequence;Peptide 36
  Unk
SRGGSQASSRSSSRS

SEQ ID NO: 43 - protein, 15 aa, Artificial Sequence;Peptide 37
  Unk
QASSRSSSRSRNSSR

SEQ ID NO: 44 - protein, 15 aa, Artificial Sequence;Peptide 38
  Unk
SSSRSRNSSRNSTPG

SEQ ID NO: 45 - protein, 15 aa, Artificial Sequence;Peptide 39
  Unk
RNSSRNSTPGSNRGT

SEQ ID NO: 46 - protein, 15 aa, Artificial Sequence;Peptide 40
  Unk
NSTPGSNRGTSPARM

SEQ ID NO: 47 - protein, 15 aa, Artificial Sequence;Peptide 41
  Unk
SNRGTSPARMAGNGG

SEQ ID NO: 48 - protein, 15 aa, Artificial Sequence;Peptide 42
  Unk
SPARMAGNGGDAALA

SEQ ID NO: 49 - protein, 15 aa, Artificial Sequence;Peptide 43
  Unk
AGNGGDAALALLLLD

SEQ ID NO: 50 - protein, 15 aa, Artificial Sequence;Peptide 44
  Unk
DAALALLLLDRLNQL

SEQ ID NO: 51 - protein, 15 aa, Artificial Sequence;Peptide 45
  Unk
LLLLDRLNQLESKMS

SEQ ID NO: 52 - protein, 15 aa, Artificial Sequence;Peptide 46
  Unk
RLNQLESKMSGKGQQ

SEQ ID NO: 53 - protein, 15 aa, Artificial Sequence;Peptide 47
  Unk
ESKMSGKGQQQQGQT

SEQ ID NO: 54 - protein, 15 aa, Artificial Sequence;Peptide 48
  Unk
GKGQQQQGQTVTKKS

SEQ ID NO: 55 - protein, 15 aa, Artificial Sequence;Peptide 49
  Unk
QQGQTVTKKSAAEAS

SEQ ID NO: 56 - protein, 15 aa, Artificial Sequence;Peptide 50
  Unk
VTKKSAAEASKKPRQ

SEQ ID NO: 57 - protein, 15 aa, Artificial Sequence;Peptide 51
  Unk
AAEASKKPRQKRTAT

SEQ ID NO: 58 - protein, 15 aa, Artificial Sequence;Peptide 52
  Unk
KKPRQKRTATKAYNV

SEQ ID NO: 59 - protein, 15 aa, Artificial Sequence;Peptide 53
  Unk
KRTATKAYNVTQAFG

SEQ ID NO: 60 - protein, 15 aa, Artificial Sequence;Peptide 54
  Unk
KAYNVTQAFGRRGPE

SEQ ID NO: 61 - protein, 15 aa, Artificial Sequence;Peptide 55
  Unk
TQAFGRRGPEQTQGN

SEQ ID NO: 62 - protein, 15 aa, Artificial Sequence;Peptide 56
  Unk
RRGPEQTQGNFGDQE

SEQ ID NO: 63 - protein, 15 aa, Artificial Sequence;Peptide 57
  Unk
QTQGNFGDQELIRQG

SEQ ID NO: 64 - protein, 15 aa, Artificial Sequence;Peptide 58
  Unk
FGDQELIRQGTDYKH

SEQ ID NO: 65 - protein, 15 aa, Artificial Sequence;Peptide 59
  Unk
LIRQGTDYKHWPQIA

SEQ ID NO: 66 - protein, 15 aa, Artificial Sequence;Peptide 60
  Unk
TDYKHWPQIAQFAPS

SEQ ID NO: 67 - protein, 15 aa, Artificial Sequence;Peptide 61
  Unk
WPQIAQFAPSASAFF

SEQ ID NO: 68 - protein, 15 aa, Artificial Sequence;Peptide 62
  Unk
QFAPSASAFFGMSRI

SEQ ID NO: 69 - protein, 15 aa, Artificial Sequence;Peptide 63
  Unk
ASAFFGMSRIGMEVT

SEQ ID NO: 70 - protein, 15 aa, Artificial Sequence;Peptide 64
  Unk
GMSRIGMEVTPSGTW

SEQ ID NO: 71 - protein, 15 aa, Artificial Sequence;Peptide 65
  Unk
GMEVTPSGTWLTYTG

SEQ ID NO: 72 - protein, 15 aa, Artificial Sequence;Peptide 66
  Unk
PSGTWLTYTGAIKLD

SEQ ID NO: 73 - protein, 15 aa, Artificial Sequence;Peptide 67
  Unk
LTYTGAIKLDDKDPN

SEQ ID NO: 74 - protein, 15 aa, Artificial Sequence;Peptide 68
  Unk
AIKLDDKDPNFKDQV

SEQ ID NO: 75 - protein, 15 aa, Artificial Sequence;Peptide 69
  Unk
DKDPNFKDQVILLNK

SEQ ID NO: 76 - protein, 15 aa, Artificial Sequence;Peptide 70
  Unk
FKDQVILLNKHIDAY

SEQ ID NO: 77 - protein, 15 aa, Artificial Sequence;Peptide 71
  Unk
ILLNKHIDAYKTFPP

SEQ ID NO: 78 - protein, 15 aa, Artificial Sequence;Peptide 72
  Unk
HIDAYKTFPPTEPKK

SEQ ID NO: 79 - protein, 15 aa, Artificial Sequence;Peptide 73
  Unk
KTFPPTEPKKDKKKK

SEQ ID NO: 80 - protein, 15 aa, Artificial Sequence;Peptide 74
  Unk
TEPKKDKKKKADETQ

SEQ ID NO: 81 - protein, 15 aa, Artificial Sequence;Peptide 75
  Unk
DKKKKADETQALPQR

SEQ ID NO: 82 - protein, 15 aa, Artificial Sequence;Peptide 76
  Unk
ADETQALPQRQKKQQ

SEQ ID NO: 83 - protein, 15 aa, Artificial Sequence;Peptide 77
  Unk
ALPQRQKKQQTVTLL

SEQ ID NO: 84 - protein, 15 aa, Artificial Sequence;Peptide 78
  Unk
QKKQQTVTLLPAADL

SEQ ID NO: 85 - protein, 15 aa, Artificial Sequence;Peptide 79
  Unk
TVTLLPAADLDDFSK

SEQ ID NO: 86 - protein, 15 aa, Artificial Sequence;Peptide 80
  Unk
PAADLDDFSKDDFSK

SEQ ID NO: 87 - protein, 15 aa, Artificial Sequence;Peptide 81
  Unk
DDFSKDDFSKMSSAD

SEQ ID NO: 88 - protein, 14 aa, Artificial Sequence;Peptide 82
  Unk
QLQQSMSSADSTQA

SEQ ID NO: 89 - DNA, 30 bp, Artificial Sequence;N fwd Primer
  Unk
gcgggccctccgataacggcccacaaaatc

SEQ ID NO: 90 - DNA, 28 bp, Artificial Sequence;N rev Primer
  Unk
gcgggcccggcctgagtactatctgcag

SEQ ID NO: 91 - protein, 15 aa, Artificial Sequence;Peptide 45 variant
  Unk
LLLLDRLNQLESKMF

SEQ ID NO: 92 - protein, 15 aa, Artificial Sequence;Peptide 45 variant
  Unk
LLLLDRLNQLESKIS

SEQ ID NO: 93 - protein, 15 aa, Artificial Sequence;Peptide 45 variant
  Unk
LLLLDRLNQFESKMS

SEQ ID NO: 94 - protein, 15 aa, Artificial Sequence;Peptide 45 variant
  Unk
LLLLDRLNHLESKMS

SEQ ID NO: 95 - protein, 9 aa, Artificial Sequence;Peptide 45 core
  Unk
LLLDRLNQL

SEQ ID NO: 96 - protein, 9 aa, Artificial Sequence;Peptide 2 core
  Unk
NQRNAPITF

SEQ ID NO: 97 - protein, 9 aa, Artificial Sequence;Peptide 9 core
  Unk
QGLPNNTSW

SEQ ID NO: 98 - protein, 9 aa, Artificial Sequence;Peptide 10 core
  Unk
NTASWFTAL

SEQ ID NO: 99 - protein, 9 aa, Artificial Sequence;Peptide 23 core
  Unk
SPRWYFYYL

SEQ ID NO: 100 - protein, 9 aa, Artificial Sequence;Peptide 30 core
  Unk
GTRNPANNA

SEQ ID NO: 101 - protein, 9 aa, Artificial Sequence;Peptide 31 core
  Unk
NNAAIVLQL

SEQ ID NO: 102 - protein, 9 aa, Artificial Sequence;Peptide 32 core
  Unk
LQLPQGTTL

SEQ ID NO: 103 - protein, 9 aa, Artificial Sequence;Peptide 61 core
  Unk
AQFPSASAF

SEQ ID NO: 104 - protein, 9 aa, Artificial Sequence;Peptide 63 core
  Unk
ASAFFGMSR

SEQ ID NO: 105 - protein, 9 aa, Artificial Sequence;Peptide 70 core
  Unk
LLNKHIDAY

SEQ ID NO: 106 - protein, 14 aa, Artificial Sequence;Peptide 22 core
  Unk
PRWYFYYLGTGPEA

SEQ ID NO: 107 - protein, 14 aa, Artificial Sequence;Peptide 61 core
  Unk
PQIAQFAPSASAFF

SEQ ID NO: 108 - protein, 14 aa, Artificial Sequence;Peptide 66 core
  Unk
PSGTWLTYTGAIKL

SEQ ID NO: 109 - DNA, 20 bp, Artificial sequence
  2019-nCoV_N1-F primer
gaccccaaaatcagcgaaat

SEQ ID NO: 110 - DNA, 24 bp, artificial sequence
  2019-nCoV_N1-R primer
tctggttactgccagttgaatctg

SEQ ID NO: 111 - DNA, 24 bp, Artificial Sequence
  2019-nCoV_N1-P probe
  misc_feature (1)..(1): 5′ FAM label
  misc_feature (24)..(24): 3′-BHQ1 quencher
accccgcattacgtttggtggacc

SEQ ID NO: 112 - DNA, 23 bp, Artificial Sequence
  sg-N-F primer
cgatctcttgtagatctgttctc

SEQ ID NO: 113 - DNA, 20 bp, Artificial Sequence
  sg-N-R primer
ggtgaaccaagacgcagtat

SEQ ID NO: 114 - DNA, 25 bp, Artificial Sequence
  Sg-N-P probe
  misc_feature (1)..(1): 5′-FAM label
  misc_feature (9)..(10): internal ZEN quencher
  misc_feature (25)..(25): 3′-3IABkFQ quencher
taaccagaatggagaacgcagtggg

SEQ ID NO: 115 - protein, 16 aa, Artificial Sequence
  Spike region analyzed for MHC class I epitope
GVYFASTEKSNIIRGW

SEQ ID NO: 116 - protein, 10 aa, Artificial Sequence
  Putative spike MHC class I epitope
TEKSNIIRGW

SEQ ID NO: 117 - protein, 9 aa, Artificial Sequence
  Putative spike MHC class I epitope
GVYFASTEK

SEQ ID NO: 118 - protein, 10 aa, Artificial Sequence
  Putative spike MHC class I epitope
DGVYFASTEK

SEQ ID NO: 119 - protein, 9 aa, Artificial Sequence
  Putative spike MHC class I epitope
STEKSNIIR

SEQ ID NO: 120 - protein, 11 aa, Artificial Sequence
  Spike region analyzed for MHC class I epitope
GVYYHKNNKSW

SEQ ID NO: 121 - protein, 9 aa, Artificial Sequence
  Putative spike MHC class I epitope
GVYYHKNNK

SEQ ID NO: 122 - protein, 9 aa, Artificial Sequence
  Putative spike MHC class I epitope
YYHKNNKSW

SEQ ID NO: 123 - protein, 10 aa, Artificial Sequence
  Putative spike MHC class I epitope
VYYHKNNKSW

SEQ ID NO: 124 - protein, 17 aa, Artificial Sequence
  Spike region analyzed for MHC class I epitope
GEVFNATRFASVYAWNR

SEQ ID NO: 125 - protein, 10 aa, Artificial Sequence
  Putative spike MHC class I epitope
ATRFASVYAW

SEQ ID NO: 126 - protein, 9 aa, Artificial Sequence
  Putative spike MHC class I epitope
NATRFASVY

SEQ ID NO: 127 - protein, 9 aa, Artificial Sequence
  Putative spike MHC class I epitope
GEVFNATRF

SEQ ID NO: 128 - protein, 9 aa, Artificial Sequence
  Putative spike MHC class I epitope
EVFNATRFA

SEQ ID NO: 129 - protein, 9 aa, Artificial Sequence
  Putative spike MHC class I epitope
ATRFASVYA

SEQ ID NO: 130 - protein, 10 aa, Artificial Sequence
  Putative spike MHC class I epitope
RFASVYAWNR

SEQ ID NO: 131 - protein, 11 aa, Artificial Sequence
  Spike region analyzed for MHC class I epitope
LYNSASFSTFK

SEQ ID NO: 132 - protein, 10 aa, Artificial Sequence
  Putative spike MHC class I epitope
LYNSASFSTF

SEQ ID NO: 133 - protein, 9 aa, Artificial Sequence
  Putative spike MHC class I epitope
NSASFSTFK

SEQ ID NO: 134 - protein, 18 aa, Artificial Sequence
  Spike region analyzed for MHC class I epitope
RQIAPGQTGKIADYNYKL

SEQ ID NO: 135 - protein, 10 aa, Artificial Sequence
  Putative spike MHC class I epitope
RQIAPGQTGK

SEQ ID NO: 136 - protein, 9 aa, Artificial Sequence
  Putative spike MHC class I epitope
KIADYNYKL

SEQ ID NO: 137 - protein, 9 aa, Artificial Sequence
  Putative spike MHC class I epitope
GQTGKIADY

SEQ ID NO: 138 - protein, 9 aa, Artificial Sequence
  Putative spike MHC class I epitope
QIAPGQTGK

SEQ ID NO: 139 - protein, 14 aa, Artificial Sequence
  Spike region analyzed for MHC class I epitope
GNYNYLYRLFRKSN

SEQ ID NO: 140 - protein, 9 aa, Artificial Sequence
  Putative spike MHC class I epitope
NYNYLYRLF

SEQ ID NO: 141 - protein, 9 aa, Artificial Sequence
  Putative spike MHC class I epitope
YNYLYRLFR

SEQ ID NO: 142 - protein, 9 aa, Artificial Sequence
  Spike region analyzed for MHC class I epitope and putative spike MHC class I epitope
YFPLQSYGF

SEQ ID NO: 143 - protein, 9 aa, Artificial Sequence
  Spike region analyzed for MHC class I epitope and Putative spike MHC class I epitope
QTNSPRRAR

SEQ ID NO: 144 - protein, 13 aa, Artificial Sequence
  Spike region analyzed for MHC class I epitope
ALNTLVKQLSSNF

SEQ ID NO: 145 - protein, 9 aa, Artificial Sequence
  Putative spike MHC class I epitope
ALNTLVKQL

SEQ ID NO: 146 - protein, 9 aa, Artificial Sequence
  Putative spike MHC class I epitope
LVKQLSSNF

SEQ ID NO: 147 - protein, 23 aa, Artificial Sequence
  Spike region analyzed for MHC class I epitope
SVLNDILSRLDKVEAEVQIDRLI

SEQ ID NO: 148 - protein, 9 aa, Artificial Sequence
  Putative spike MHC class I epitope
VLNDILSRL

SEQ ID NO: 149 - protein, 9 aa, Artificial Sequence
  Putative spike MHC class I epitope
SVLNDILSR

SEQ ID NO: 150 - protein, 10 aa, Artificial Sequence
  Putative spike MHC class I epitope
SVLNDILSRL

SEQ ID NO: 151 - protein, 10 aa, Artificial Sequence
  Nucleocapsid region analyzed for MHC class I epitope and putative MHC class I epitope
MSDNGPQNQR

SEQ ID NO: 152 - protein, 9 aa, Artificial Sequence
  Spike MHC class II core epitope
FSNVTWFHA

SEQ ID NO: 153 - protein, 9 aa, Artificial Sequence
  Spike MHC class II core epitope
LVRDLPQGF

SEQ ID NO: 154 - protein, 9 aa, Artificial Sequence
  Spike MHC class II core epitope
FQTLLALHR

SEQ ID NO: 155 - protein, 9 aa, Artificial Sequence
  Spike MHC class II core epitope
LALHRSYLT

SEQ ID NO: 156 - protein, 9 aa, Artificial Sequence
  Spike MHC class II core epitope
VLYNSASFS

SEQ ID NO: 157 - protein, 9 aa, Artificial Sequence
  Spike MHC class II core epitope
LYRLFRKSN

SEQ ID NO: 158 - protein, 9 aa, Artificial Sequence
  Spike MHC class II core epitope
LTGTGVLTE

SEQ ID NO: 159 - protein, 9 aa, Artificial Sequence
  Spike MHC class II core epitope
IAIPTNFTI

SEQ ID NO: 160 - protein, 9 aa, Artificial Sequence
  Spike MHC class II core epitope
LVKQLSSNF